(19) United States
(12) Patent Application Publication
(10) Pub. No.: US 2026/0245658 A1
(43) Pub. Date: Aug. 20, 2026

(54) METHODS AND SYSTEMS FOR POSITIVE CFDNA SCREENING ON GENETIC VARIATIONS USING MOSAICISM RATIO

(71) Applicant: Laboratory Corporation of America Holdings, Burlington, NC (US)

(72) Inventors: Erica Soster, San Diego, CA (US); Brittany Dyr, San Diego, CA (US); Jill Rafalko, San Diego, CA (US); Eyad Almasri, San Diego, CA (US); Phillip Cacheris, San Diego, CA (US)

(73) Assignee: Laboratory Corporation of America Holdings, Burlington, NC (US)

(21) Appl. No.: 19/161,983

(22) PCT Filed: Mar. 4, 2024
(86) PCT No.: PCT/US2024/018423

§ 371 Date: Sep. 3, 2025

Related U.S. Application Data
(60) Provisional application No. 63/488,395, filed on Mar. 3, 2023.

(51) Int. Cl. G16B 20/20; C12Q 1/6806; C12Q 1/6883; C12Q 1/6886

(52) U.S. Cl. CPC G16B 20/20; C12Q 1/6806; C12Q 1/6883; C12Q 1/6886; C12Q 2600/156

(57) ABSTRACT

Technology provided herein relates in part to determine an origin of a genetic variation in a biological sample from a subject and determining a size and location of the genetic variation. In particular, a method is provided to obtain circulating cell free nucleic acid in the biological sample; sequence the circulating cell free nucleic acid to obtain sequencing data; identify a presence or absence of the genetic variation; identify one or more metrics for the genetic variation based on the sequencing data; determine a mosaicism ratio for the genetic variation in the biological sample based on a fraction of nucleic acid having the genetic variation and a fraction of minor nucleic acid; classify the origin of the genetic variation based on the mosaicism ratio; and generate a report concerning a recommendation of a follow-up screening.

### CROSS-REFERENCE TO RELATED APPLICATION

The present application claims priority and benefit from U.S. Provisional Application No. 63/488,395, filed Mar. 3, 2023, the entire contents of which are incorporated herein by reference for all purposes.

### FIELD

Technology provided herein relates in part to methods, systems, machines and computer program products for non-invasive classification of a mosaic copy number variation (CNV) for a test sample. Technology provided herein is useful for classifying a mosaic CNV for a sample as part of non-invasive pre-natal testing (NIPT) and oncology testing, for example. Technology provided herein is also useful for prediction of deletion size, location, and origin of a CNV and providing appropriate follow-up for the test sample.

### BACKGROUND

Genetic information of living organisms (e.g., animals, plants and microorganisms) and other forms of replicating genetic information (e.g., viruses) is encoded in deoxyribonucleic acid (DNA) or ribonucleic acid (RNA). Genetic information is a succession of nucleotides or modified nucleotides representing the primary structure of chemical or hypothetical nucleic acids. In humans, the complete genome contains about 30,000 genes located on 24 chromosomes (i.e., 22 autosomes, an X chromosome and a Y chromosome; see The Human Genome, T. Strachan, BIOS Scientific Publishers, 1992). Each gene encodes a specific protein, which after expression via transcription and translation fulfills a specific biochemical function within a living cell.

Many medical conditions are caused by one or more genetic variations and/or genetic alterations. Certain genetic variations and/or genetic alterations cause medical conditions that include, for example, hemophilia, thalassemia, Duchenne Muscular Dystrophy (DMD), Huntington's Disease (HD), Alzheimer's Disease and Cystic Fibrosis (CF) (Human Genome Mutations, D. N. Cooper and M. Krawczak, BIOS Publishers, 1993). Such genetic diseases can result from an addition, substitution, or deletion of a single nucleotide in DNA of a particular gene. Certain birth defects are caused by a chromosomal abnormality, also referred to as an aneuploidy, such as Trisomy 21 (Down's Syndrome), Trisomy 13 (Patau Syndrome), Trisomy 18 (Edward's Syndrome), Monosomy X (Turner's Syndrome) and certain sex chromosome aneuploidies such as Klinefelter's Syndrome (XXY), for example. Another genetic variation is fetal sex, which can often be determined based on sex chromosomes X and Y. Some genetic variations may predispose an individual to, or cause, any of a number of diseases such as, for example, diabetes, arteriosclerosis, obesity, various autoimmune diseases and a cell proliferative disorder such as a cancer, tumor, neoplasm, metastatic disease, the like or combination thereof. A cancer, tumor, neoplasm, or metastatic disease sometimes is a disorder or condition of the liver, lung, spleen, pancreas, colon, skin, bladder, eye, brain, esophagus, head, neck, ovary, testes, prostate, the like or combination thereof.

Identifying one or more genetic variations and/or genetic alterations (e.g., copy number alterations, copy number variations, single nucleotide alterations, single nucleotide variations, chromosome alterations, translocations, deletions, insertions, and the like) or variances can lead to diagnosis of, or determining predisposition to, a particular medical condition. Identifying a genetic variance can result in facilitating a medical decision and/or employing a helpful medical procedure. In certain embodiments, identification of one or more genetic variations and/or genetic alterations involves the analysis of circulating cell-free nucleic acid. Circulating cell-free nucleic acid (CCF-NA), such as cell-free DNA (cfDNA) for example, is composed of DNA fragments that originate from cell death and circulate in peripheral blood. High concentrations of cfDNA can be indicative of certain clinical conditions such as cancer, trauma, burns, myocardial infarction, stroke, sepsis, infection, and other illnesses. Additionally, cell-free fetal DNA (cffDNA) can be detected in the maternal bloodstream and used for various non-invasive prenatal diagnostics.

### BRIEF SUMMARY

Techniques described herein are methods and systems for determining an origin of a genetic variation in a biological sample from a subject and determining a size and location of the genetic variation. In various embodiments, a method is provided that comprises: obtaining circulating cell free nucleic acid in the biological sample from the subject, wherein the biological sample has been identified as having the genetic variation, and the circulating cell free nucleic acid comprises major nucleic acid and minor nucleic acid; sequencing, using a massively parallel sequencing (MPS) platform, the circulating cell free nucleic acid to obtain sequencing data; identifying, by a computing system, one or more metrics for the genetic variation based on the sequencing data, wherein the one or more metrics comprise a size of the genetic variation and/or a breakpoint of the genetic variation; determining, by the computing system, a mosaicism ratio for the genetic variation in the biological sample, wherein the determining the mosaicism ratio comprises: determining, by the computing system, a fraction of nucleic acid having the genetic variation in the circulating cell free nucleic acid based on the sequencing data; determining, by the computing system, a fraction of the minor nucleic acid in the circulating cell free nucleic acid based on the sequencing data; and generating, by the computing system, the mosaicism ratio using the fraction of nucleic acid having the genetic variation in the circulating cell free nucleic acid to the fraction of the minor nucleic acid in the circulating cell free nucleic acid; classifying, by the computing system, the origin of the genetic variation based on the mosaicism ratio, wherein the origin of the genetic variation is major when the mosaicism ratio is greater than or equal to a first threshold, and the origin of the genetic variation is minor when the mosaicism ratio is less than a second threshold; and generating, by the computing system, a report concerning a recommendation of a follow-up screening, wherein the recommendation is based on the one or more metrics and the origin of the genetic variation.

In some embodiments, the genetic variation is an aneuploidy, a genome-wide copy number variation, a single gene disorder, or a microdeletion.

In some embodiments, the genetic variation is a 22q11.2 deletion, a trisomy 21 (T21), a trisomy 18 (T18), or a trisomy 13 (T13).

In some embodiments, the subject is a pregnant subject, the major nucleic acid is maternal nucleic acid, and the minor nucleic acid is fetal nucleic acid.

In some embodiments, the subject is a transplant patient, the major nucleic acid is recipient nucleic acid, and the minor nucleic acid is donor nucleic acid.

In some embodiments, the subject is a tumor patient, the major nucleic acid is normal nucleic acid, and the minor nucleic acid is tumor nucleic acid.

In some embodiments, the first threshold is 3.

In some embodiments, when the origin of the genetic variation is classified to be maternal when the mosaicism ratio is greater than or equal to 3, and when the genetic variation is an atypical or nested deletion, the recommendation is not to perform a fluorescent in situ hybridization (FISH) test.

In some embodiments, when the origin of the genetic variation is classified to be maternal when the mosaicism ratio is greater than or equal to 3, and when the genetic variation is an atypical or nested deletion, the recommendation is to perform a microarray analysis test.

In some embodiments, the first threshold and the second threshold are the same.

In some embodiments, the method further comprises determining, by the computing system, a presence of the genetic variation.

In some embodiments, the presence of the genetic variation is determined by a particular screening method.

In some embodiments, the presence of the genetic variation is determined based on the sequencing data.

In some embodiments, the method further comprises administering a particular screening test to the subject based on the report.

In some embodiments, the method further comprises enriching the minor nucleic acid in the biological sample before sequencing.

In some embodiments, a system is provided that includes one or more data processors and a non-transitory computer readable medium containing instructions which, when executed on the one or more data processors, cause the one or more data processors to perform part or all of one or more methods or processes disclosed herein.

In some embodiments, a computer-program product is provided that is tangibly embodied in a non-transitory machine-readable medium and that includes instructions configured to cause one or more data processors to perform part or all of one or more methods disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the techniques claimed. Thus, it should be understood that although the present techniques have been specifically disclosed by embodiments and optional features, modification and variation of the concepts herein disclosed may be resorted to by those skilled in the art, and that such modifications and variations are considered to be within the scope of the techniques as defined by the appended claims.

### BRIEF DESCRIPTION OF THE DRAWINGS

The drawings illustrate certain embodiments of the technology and are not limiting. For clarity and ease of illustration, the drawings are not made to scale and, in some instances, various aspects may be shown exaggerated or enlarged to facilitate an understanding of particular embodiments.

FIG. 1 is a block diagram of a clinical test pipeline that implements a NIPT procedure or assay for analyzing nucleic acids in accordance with various embodiments.

FIG. 2 is a flowchart illustrating a process for classifying an origin of a genetic variation in a biological sample in accordance with various embodiments.

FIG. 3 is a flowchart illustrating a process for identifying a genetic variation in a biological sample and classifying an origin of the genetic variation in accordance with various embodiments.

FIG. 4 shows an illustrative embodiment of a system in which various embodiments of the technology may be implemented in accordance with various embodiments.

FIG. 5 shows indication for testing in accordance with various embodiments.

FIG. 6 shows a comparison of sequencing traces for a suspected maternal deletion (A) and suspected fetal deletion (B) in accordance with various embodiments.

FIG. 7A is a box and whisker plot showing the separation of the MR of suspected fetal and suspected maternal cases from cfDNA in accordance with various embodiments.

FIG. 7B is a box and whisker plot showing estimated deletion sizes for suspected fetal events as compared to suspected maternal events from cfDNA in accordance with various embodiments.

FIG. 8 shows a plot of the start and end breakpoints predicted from cfDNA, with groupings of frequent deletions of similar sizes, the common, larger deletions toward the bottom of the figure and the smaller nested/atypical deletions toward the top, in accordance with various embodiments.

FIG. 9A is a box and whisker plot showing the comparison of deletion size for all cases with and without ultrasound findings in accordance with various embodiments.

FIG. 9B is a box and whisker plot showing the comparison of deletion size for suspected fetal cases with and without ultrasound findings in accordance with various embodiments.

FIG. 9C is a box and whisker plot showing the comparison of deletion size for confirmed fetal cases with and without ultrasound findings in accordance with various embodiments.

FIG. 10 is a flow chart summarizing the diagnostic testing for the 194 cases with diagnostic follow-up in accordance with various embodiments.

FIG. 11 is a scatterplot showing the relationship between estimated deletion size on cfDNA and confirmed deletion size on microarray in accordance with various embodiments.

In the appended figures, similar components and/or features can have the same reference label. Further, various components of the same type can be distinguished by following the reference label by a dash and a second label that distinguishes among the similar components. If only the first reference label is used in the specification, the description is applicable to any one of the similar components having the same first reference label irrespective of the second reference label.

### DETAILED DESCRIPTION

The ensuing description provides preferred exemplary embodiments only, and is not intended to limit the scope, applicability or configuration of the disclosure. Rather, the ensuing description of the preferred exemplary embodiments will provide those skilled in the art with an enabling description for implementing various embodiments. It is understood that various changes may be made in the function and arrangement of elements without departing from the spirit and scope as set forth in the appended claims.

Specific details are given in the following description to provide a thorough understanding of the embodiments. However, it will be understood that the embodiments may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

Also, it is noted that individual embodiments may be described as a process which is depicted as a flowchart, a flow diagram, a data flow diagram, a structure diagram, or a block diagram. Although a flowchart or diagram may describe the operations as a sequential process, many of the operations may be performed in parallel or concurrently. In addition, the order of the operations may be re-arranged. A process is terminated when its operations are completed, but could have additional steps not included in a figure. A process may correspond to a method, a function, a procedure, a subroutine, a subprogram, etc. When a process corresponds to a function, its termination may correspond to a return of the function to the calling function or the main function.

### A. INTRODUCTION

The analysis of cell-free nucleic acid in fluid samples, particularly from pregnant individuals, holds significant promise for non-invasive prenatal testing (NIPT). Cell-free nucleic acid screening or NIPT is a screening test that utilizes bioinformatic tools and processes and next generation sequencing of fragments of DNA in maternal serum to determine the probability of certain chromosome conditions in a pregnancy. Using bioinformatic tools and next-generation sequencing, technologies have been developed to examine fragments of DNA in maternal serum to determine the likelihood of certain genetic variation. This screening method can be commonly used to detect trisomy 21, trisomy 18, and trisomy 13, and it can also identify fetal aneuploidy, other aneuploidies, triploids, and specific microdeletion conditions. An abnormal result suggests an increased risk for a particular condition, but diagnostic testing (such as amniocentesis) may be needed to confirm the diagnosis. Although early screening primarily focused on common aneuploidies, recent advances have expanded the scope of non-invasive prenatal screening to include genome-wide copy number variants, single gene disorders, and select microdeletion syndromes such as 22q11.2 deletion syndrome (22q11.2DS).

22q11.2DS, also known as DiGeorge syndrome or velocardiofacial syndrome (VCFS), is a condition associated with a wide range of anomalies, including cardiac defects, immune system dysfunction, oral clefting, hypocalcemia, developmental delays, and behavioral complications. It has a reported prevalence of 1 in 1,000 in unselected fetuses or 1 in 3,000-6,000 live births. Despite becoming clinically available in 2013, screening for 22q11.2DS using cfDNA is not routinely supported during pregnancy by most professional societies due to a lack of clinical data, as well as the perception of a reduced positive predictive values (PPV) ranging from 18% to greater than 97% associated with a positive screening result. Nevertheless, the ability to identify 22q11.2DS prenatally will allow parents to make informed reproductive choices and may benefit both perinatal management and maternal health (e.g., in an event of a maternally inherited deletion). Additionally, many cases of 22q11.2DS may not otherwise be identified in the prenatal or neonatal period via ultrasound or routine assessment of the patient's family or obstetric history, as the condition often occurs de novo and is not associated with maternal age, and affected fetuses may or may not present with ultrasound findings.

Further challenges exist regarding screening for 22q11.2DS by cfDNA due to the inherent biological and technical limitations. For example, a variety of deletion sizes have been reported with 22q11.2DS owing to the low copy number repeats (LCRs), labeled A through D, in the genetic variation area. Approximately 85% of patients have an about 2.54 Mb sized deletion, which has been frequently described as a “3 Mb” deletion, from A-D LCRs. The remaining ~15% have smaller atypical or nested deletions (for example, involving the A-B LCRs or C-D LCRs) which may be associated with a milder phenotype and reduced penetrance. This region is particularly susceptible to meiotic error due to the significant homology between these LCRs; non-allelic homologous recombination leads to both the “3 Mb” deletion as well as the smaller, nested deletions. Given the size of 22q11.2 deletions, even the typical “3 Mb” deletion will be below the resolution of detection by routine prenatal chromosome analysis; diagnostic confirmation of 22q11.2 deletions typically requires microarray and/or fluorescent in situ hybridization (FISH). Additionally, common FISH probes for detecting 22q11.2 deletions only hybridize between the A-B LCRs and are therefore not useful for detecting atypical nested deletions clinically.

Another major concern is that cfDNA screening utilizes maternal plasma containing both fetal and maternal cfDNA. Therefore, the presence of a maternal genetic variation may preclude the fetal assessment of the genetic variation. Even though fetal assessment using cfDNA may not be possible when a maternal genetic variation is suspected, if the origin of the genetic variation (i.e., maternal or fetal) can be detected and reported, it may suggest a percentage of risk projected to an affected fetus. For example, a suspected maternal 22q11.2 deletion suggests a 50% risk for an affected fetus.

The limitations and challenges described with respect to the 22q11.2DS example and the NIPT screening above illustrate the need of developing clinically reliable methods and systems for detecting a genetic variation in a subject using circulating cell-free DNA of the subject. To address these limitations, challenges, and problems, techniques disclosed herein are directed to methods and systems using cfDNA and biological metrics to detect a size and location of a genetic variation, classify an origin of the genetic variation, and provide a recommendation concerning a follow-up. The methods and systems are related to and utilize next generation sequencing technology such as massively parallel sequencing (MPS). The disclosed methods and systems can be used de novo to detect the genetic variation and its associated indexes (e.g., size, location), and they can also be used to confirm the existence of the genetic variation in the sample and provide the origin of the genetic variation. The disclosed methods and systems improve the non-invasive prenatal screening technology by providing more accurate identification of genetic variations, classifying a suspect origin of the genetic variations, and allowing patients to make informed decisions. The disclosed methods and systems also improve follow-up recommendations by providing a comprehensive report that links the genetic variation information to the follow-up recommendations in an intuitive and comprehensible graphical user interface.

The disclosed techniques can also be used in areas besides NIPT, for example, in the area of organ transplantation. In such scenarios, the disclosed techniques can be used to monitor transplant outcomes, allowing for early intervention and personalized treatment.

The disclosed techniques offer significant commercial advantages by providing detailed information about the origin of genetic variations and generating comprehensive diagnostic reports. These techniques leverage sequencing data obtained through next generation sequencing (e.g., massively parallel sequencing, which allows for the simultaneous analysis of millions of DNA fragments) which is known for its efficiency in terms of time and cost when compared to screening methods like microarray (e.g., involving the use of thousands of DNA probes to detect genetic variations) or fluorescence in situ hybridization (FISH, involving the use of fluorescent probes to visualize specific DNA sequences).

In an illustrative embodiment, a method is provided that comprises: obtaining circulating cell free nucleic acid in a biological sample from a subject, wherein the circulating cell free nucleic acid comprises major nucleic acid and minor nucleic acid; sequencing, using a massively parallel sequencing (MPS) platform, the circulating cell free nucleic acid to obtain sequencing data; identifying, by a computing system, an existence of the genetic variation based on the sequencing data; identifying, by the computing system, one or more metrics for the genetic variation based on the sequencing data, wherein the one or more metrics comprise a size of the genetic variation and/or a breakpoint of the genetic variation; determining, by the computing system, a mosaicism ratio for the genetic variation in the biological sample, wherein the determining the mosaicism ratio comprises: determining, by the computing system, a fraction of nucleic acid having the genetic variation in the circulating cell free nucleic acid based on the sequencing data; determining, by the computing system, a fraction of the minor nucleic acid in the circulating cell free nucleic acid based on the sequencing data; and generating, by the computing system, the mosaicism ratio using the fraction of nucleic acid having the genetic variation in the circulating cell free nucleic acid to the fraction of the minor nucleic acid in the circulating cell free nucleic acid; classifying, by the computing system, the origin of the genetic variation based on the mosaicism ratio, wherein the origin of the genetic variation is major when the mosaicism ratio is greater than or equal to a first threshold, and the origin of the genetic variation is minor when the mosaicism ratio is less than a second threshold; and generating, by the computing system, a report concerning a recommendation of a follow-up screening, wherein the recommendation is based on the one or more metrics and the origin of the genetic variation.

As used herein, the term “origin” means the definitive origin or the suspected origin of the genetic variation. As used herein, the term “suspected” is defined as being what is believed to be true within a reasonable degree of certainty as understood by one of ordinary skill in the art (e.g., greater than 80% chance that what is believed to be true is definitively true). The reasonable degree of certainty can be exactly or about 50%, 55%, 60%, 65%, 70%, 75%, 80%, 85%, 90%, or 95%.

As used herein, the term “metric” means the actual metric(s) or the estimated metric(s) for the genetic variation. As used herein, the term “estimated” metric(s) is defined as being approximately or an approximation of a value that is used to characterize the genetic variation.

As used herein, the term “size” means the actual size or the estimated size for the genetic variation. As used herein, the term “estimated” size is defined as being approximately or an approximation of the size of the genetic variation.

As used herein, the term “major nucleic acid” means that the percentage of a subset of nucleic acid is greater than 50%, and the term “minor nucleic acid” means that the percentage of another subset of nucleic acid is less than 50%. In the context that a biological sample is obtained from a pregnant woman, the major nucleic acid generally refers to the maternal nucleic acid and the minor nucleic acid is the fetal nucleic acid. In the context that the biological sample is obtained from a post-transplant subject, the major nucleic acid generally refers to the subject nucleic acid or the recipient nucleic acid, and the minor nucleic acid is the donor (organ-donor) nucleic acid.

As used herein, the term “fetal” or “maternal” used with respect to “origin” means the origin is definitively “fetal” or “maternal”; or likely “fetal” or “maternal”. As used herein, the term “likely” is defined as being what is believed to be true within a reasonable degree of certainty as understood by one of ordinary skill in the art (e.g., greater than 80% chance that what is believed to be true is definitively true). The reasonable degree of certainty can be exactly or about 50%, 55%, 60%, 65%, 70%, 75%, 80%, 85%, 90%, or 95%.

As used herein, the terms “fetal DNA,” “fetal nucleic acid,” and “fetal cfDNA” are defined as being largely but not necessarily wholly what is specified (and include wholly what is specified) as understood by one of ordinary skill in the art. In any disclosed embodiment, the term “fetal DNA,” “fetal nucleic acid,” and “fetal cfDNA” may be understood as “placental DNA,” “placental nucleic acid,” and “placental cfDNA.” A “fetal” genetic material may be used interchangeably with a “placental” genetic material. “Fetal” nucleic acid may be used interchangeably with “minor” nucleic acid and “maternal” nucleic acid may be used interchangeably with “major” nucleic acid.

As used herein, the terms “substantially,” “approximately” and “about” are defined as being largely but not necessarily wholly what is specified (and include wholly what is specified) as understood by one of ordinary skill in the art. In any disclosed embodiment, the term “substantially,” “approximately,” or “about” may be substituted with “within [a percentage] of” what is specified, where the percentage includes 0.1, 1, 5, and 10 percent. As used herein, when an action is “based on” something, this means the action is based at least in part on at least a part of the something.

As used herein, the term “result” or “laboratory test result” means a value determined as a result of performing a single laboratory test or a score determine as a result of performing one or more laboratory tests. In some instances, the value is numerical such as 1.0, 2.5, 19.7, 25.0, etc. In other instances, the value is alpha such as negative, abnormal, positive, normal, etc. In other instances, the value is alpha numeric such as abnormal between 2.0 and 5.0 or the like.

As used herein, the terms “a set of,” “a subset of,” and “an nth set of” (e.g., “a first set of,” “a second set of”) are defined as being largely but not necessarily wholly what is specified (and include wholly what is specified) as understood by one of ordinary skill in the art. In any disclosed embodiment, the term “a set of,” “a subset of,” or “an nth set of” may be substituted with “a part of” or “a section of” what is specified, where the part or section includes an empty part and a whole part.

### B. GENETIC VARIATIONS

The presence or absence of a genetic variation can be determined using a method or apparatus described herein. A genetic variation also may be referred to as a genetic alteration, and the terms are often used interchangeably herein and in the art. In certain instances, “genetic alteration” may be used to describe a somatic alteration whereby the genome in a subset of cells in a subject contains the alteration (such as, for example, in tumor or cancer cells). In certain instances, “genetic variation” may be used to describe a variation inherited from one or both parents (such as, for example, a genetic variation in a fetus).

In certain embodiments, the presence or absence of one or more genetic variations or genetic alterations is determined according to an outcome provided by methods and apparatuses described herein. A genetic variation generally is a particular genetic phenotype present in certain individuals, and often a genetic variation is present in a statistically significant sub-population of individuals. In some embodiments, a genetic variation or genetic alteration is a chromosome abnormality or copy number alteration (e.g., aneuploidy, duplication of one or more chromosomes, loss of one or more chromosomes, partial chromosome abnormality or mosaicism (e.g., loss or gain of one or more regions of a chromosome), translocation, inversion, each of which is described in greater detail herein). Non-limiting examples of genetic variations/genetic alterations include one or more copy number alterations/variations, deletions (e.g., microdeletions), duplications (e.g., microduplications), insertions, mutations (e.g., single nucleotide variations, single nucleotide alterations), polymorphisms (e.g., single-nucleotide polymorphisms), fusions, repeats (e.g., short tandem repeats), distinct methylation sites, distinct methylation patterns, the like and combinations thereof. An insertion, repeat, deletion, duplication, mutation or polymorphism can be of any length, and in some embodiments, is about 1 base or base pair (bp) to about 250 megabases (Mb) in length. In some embodiments, an insertion, repeat, deletion, duplication, mutation or polymorphism is about 1 base or base pair (bp) to about 50,000 kilobases (kb) in length (e.g., about 10 bp, 50 bp, 100 bp, 500 bp, 1 kb, 5 kb, 10 kb, 50 kb, 100 kb, 500 kb, 1000 kb, 5000 kb or 10,000 kb in length).

A genetic variation or genetic alteration is sometimes a deletion. In certain instances, a deletion is a mutation (e.g., a genetic aberration) in which a part of a chromosome or a sequence of DNA is missing. A deletion is often the loss of genetic material. Any number of nucleotides can be deleted. A deletion can comprise the deletion of one or more entire chromosomes, a region of a chromosome, an allele, a gene, an intron, an exon, any non-coding region, any coding region, a part thereof or combination thereof. A deletion can comprise a microdeletion. A deletion can comprise the deletion of a single base.

A genetic variation or genetic alteration is sometimes a duplication. In certain instances, a duplication is a mutation (e.g., a genetic aberration) in which a part of a chromosome or a sequence of DNA is copied and inserted back into the genome. In certain embodiments, a genetic duplication (e.g., duplication) is any duplication of a region of DNA. In some embodiments, a duplication is a nucleic acid sequence that is repeated, often in tandem, within a genome or chromosome. In some embodiments, a duplication can comprise a copy of one or more entire chromosomes, a region of a chromosome, an allele, a gene, an intron, an exon, any non-coding region, any coding region, part thereof or combination thereof. A duplication can comprise a microduplication. A duplication sometimes comprises one or more copies of a duplicated nucleic acid. A duplication sometimes is characterized as a genetic region repeated one or more times (e.g., repeated 1, 2, 3, 4, 5, 6, 7, 8, 9 or 10 times). Duplications can range from small regions (thousands of base pairs) to whole chromosomes in some instances. Duplications frequently occur as the result of an error in homologous recombination or due to a retrotransposon event. Duplications have been associated with certain types of proliferative diseases. Duplications can be characterized using genomic microarrays or comparative genetic hybridization (CGH).

A genetic variation or genetic alteration is sometimes an insertion. An insertion is sometimes the addition of one or more nucleotide base pairs into a nucleic acid sequence. An insertion is sometimes a microinsertion. In certain embodiments, an insertion comprises the addition of a region of a chromosome into a genome, chromosome, or part thereof. In certain embodiments, an insertion comprises the addition of an allele, a gene, an intron, an exon, any non-coding region, any coding region, part thereof or combination thereof into a genome or part thereof. In certain embodiments, an insertion comprises the addition (e.g., insertion) of nucleic acid of unknown origin into a genome, chromosome, or part thereof. In certain embodiments, an insertion comprises the addition (e.g., insertion) of a single base.

As used herein a “copy number alteration” generally is a class or type of genetic variation, genetic alteration or chromosomal aberration. A copy number alteration also may be referred to as a copy number variation, and the terms are often used interchangeably herein and in the art. In certain instances, “copy number alteration” may be used to describe a somatic alteration whereby the genome in a subset of cells in a subject contains the alteration (such as, for example, in tumor or cancer cells). In certain instances, “copy number variation” may be used to describe a variation inherited from one or both parents (such as, for example, a copy number variation in a fetus). A copy number alteration can be a deletion (e.g., microdeletion), duplication (e.g., a microduplication) or insertion (e.g., a microinsertion). Often, the prefix “micro” as used herein sometimes is a region of nucleic acid less than 5 Mb in length. A copy number alteration can include one or more deletions (e.g., microdeletion), duplications and/or insertions (e.g., a microduplication, microinsertion) of a part of a chromosome. In certain embodiments, a duplication comprises an insertion. In certain embodiments, an insertion is a duplication. In certain embodiments, an insertion is not a duplication.

In some embodiments, a copy number alteration is a copy number alteration from a tumor or cancer cell. In some embodiments, a copy number alteration is a copy number alteration from a non-cancer cell. In certain embodiments, a copy number alteration is a copy number alteration within the genome of a subject (e.g., a cancer patient) and/or within the genome of a cancer cell or tumor in a subject. A copy number alteration can be a heterozygous copy number alteration where the variation (e.g., a duplication or deletion) is present on one allele of a genome. A copy number alteration can be a homozygous copy number alteration where the alteration is present on both alleles of a genome. In some embodiments, a copy number alteration is a heterozygous or homozygous copy number alteration. In some embodiments, a copy number alteration is a heterozygous or homozygous copy number alteration from a cancer cell or non-cancer cell. A copy number alteration sometimes is present in a cancer cell genome and a non-cancer cell genome, a cancer cell genome and not a non-cancer cell genome, or a non-cancer cell genome and not a cancer cell genome.

In some embodiments, a copy number alteration is a fetal copy number alteration. Often, a fetal copy number alteration is a copy number alteration in the genome of a fetus. In some embodiments, a copy number alteration is a maternal and/or fetal copy number alteration. In certain embodiments, a maternal and/or fetal copy number alteration is a copy number alteration within the genome of a pregnant female (e.g., a female subject bearing a fetus), a female subject that gave birth or a female capable of bearing a fetus. A copy number alteration can be a heterozygous copy number alteration where the alteration (e.g., a duplication or deletion) is present on one allele of a genome. A copy number alteration can be a homozygous copy number alteration where the alteration is present on both alleles of a genome. In some embodiments, a copy number alteration is a heterozygous or homozygous fetal copy number alteration. In some embodiments, a copy number alteration is a heterozygous or homozygous maternal and/or fetal copy number alteration. A copy number alteration sometimes is present in a maternal genome and a fetal genome, a maternal genome and not a fetal genome, or a fetal genome and not a maternal genome.

“Ploidy” is a reference to the number of chromosomes present in a subject. In certain embodiments, “ploidy” is the same as “chromosome ploidy.” In humans, for example, autosomal chromosomes are often present in pairs. For example, in the absence of a genetic variation or genetic alteration, most humans have two of each autosomal chromosome (e.g., chromosomes 1-22). The presence of the normal complement of 2 autosomal chromosomes in a human is often referred to as euploid or diploid. “Microploidy” is similar in meaning to ploidy. “Microploidy” often refers to the ploidy of a part of a chromosome. The term “microploidy” sometimes is a reference to the presence or absence of a copy number alteration (e.g., a deletion, duplication and/or an insertion) within a chromosome (e.g., a homozygous or heterozygous deletion, duplication, or insertion, the like or absence thereof).

A genetic variation or genetic alteration for which the presence or absence is identified for a subject is associated with a medical condition in certain embodiments. Thus, technology described herein can be used to identify the presence or absence of one or more genetic variations or genetic alterations that are associated with a medical condition or medical state. Non-limiting examples of medical conditions include those associated with intellectual disability (e.g., Down Syndrome), aberrant cell-proliferation (e.g., cancer), presence of a micro-organism nucleic acid (e.g., virus, bacterium, fungus, yeast), and preeclampsia.

Non-limiting examples of genetic variations/genetic alterations, medical conditions and states are described hereafter.

### 1) Chromosome Abnormalities

In some embodiments, the presence or absence of a chromosome abnormality can be determined by using a method and/or apparatus described herein. Chromosome abnormalities include, without limitation, copy number alterations, and a gain or loss of an entire chromosome or a region of a chromosome comprising one or more genes. Chromosome abnormalities include monosomies, trisomies, polysomies, loss of heterozygosity, translocations, deletions and/or duplications of one or more nucleotide sequences (e.g., one or more genes), including deletions and duplications caused by unbalanced translocations. The term “chromosomal abnormality” or “aneuploidy” as used herein refer to a deviation between the structure of the subject chromosome and a normal homologous chromosome. The term “normal” refers to the predominate karyotype or banding pattern found in healthy individuals of a particular species, for example, a euploid genome (e.g., diploid in humans, e.g., 46,XX or 46,XY). As different organisms have widely varying chromosome complements, the term “aneuploidy” does not refer to a particular number of chromosomes, but rather to the situation in which the chromosome content within a given cell or cells of an organism is abnormal. In some embodiments, the term “aneuploidy” herein refers to an imbalance of genetic material caused by a loss or gain of a whole chromosome, or part of a chromosome. An “aneuploidy” can refer to one or more deletions and/or insertions of a region of a chromosome. The term “euploid,” in some embodiments, refers a normal complement of chromosomes.

The term “monosomy” as used herein refers to lack of one chromosome of the normal complement. Partial monosomy can occur in unbalanced translocations or deletions, in which only a part of the chromosome is present in a single copy. Monosomy of sex chromosomes (45, X) causes Turner syndrome, for example. The term “disomy” refers to the presence of two copies of a chromosome. For organisms such as humans that have two copies of each chromosome (those that are diploid or “euploid”), disomy is the normal condition. For organisms that normally have three or more copies of each chromosome (those that are triploid or above), disomy is an aneuploid chromosome state. In uniparental disomy, both copies of a chromosome come from the same parent (with no contribution from the other parent).

The term “trisomy” as used herein refers to the presence of three copies, instead of two copies, of a particular chromosome. The presence of an extra chromosome 21, which is found in human Down syndrome, is referred to as “Trisomy 21.” Trisomy 18 and Trisomy 13 are two other human autosomal trisomies. Trisomy of sex chromosomes can be seen in females (e.g., 47, XXX in Triple X Syndrome) or males (e.g., 47, XXY in Klinefelter's Syndrome; or 47,XYY in Jacobs Syndrome). In some embodiments, a trisomy is a duplication of most or all of an autosome. In certain embodiments, a trisomy is a whole chromosome aneuploidy resulting in three instances (e.g., three copies) of a particular type of chromosome (e.g., instead of two instances (e.g., a pair) of a particular type of chromosome for a euploid).

The terms “tetrasomy” and “pentasomy” as used herein refer to the presence of four or five copies of a chromosome, respectively. Although rarely seen with autosomes, sex chromosome tetrasomy and pentasomy have been reported in humans, including XXXX, XXXY, XXYY, XYYY, XXXXX, XXXXY, XXXYY, XXYYY and XYYYY.

In some embodiments, the disclosure provides a method of determining whether a test sample comprises trisomies, e.g., trisomy 21, trisomy 18 or trisomy 13. The method comprises providing a set of genomic portions each coupled to a copy number alteration quantification for the test sample. The genomic portions comprise portions of a reference genome to which sequence reads obtained for sample nucleic acid from the test subject have been mapped, and the copy number alteration quantification coupled to each genomic portion has been determined from a quantification of sequence reads mapped to the genomic portion. The method further comprises filtering, by a computing device, from the set of genomic portions: (i) a first subset of portions associated with copy number alterations consistently presented in a reference set of samples, and/or (ii) a second subset of portions other than representative portions derived from groups of portions identified by a clustering process applied to a reference set of samples, thereby generating a set of filtered genomic portions not containing the first subset of portions and/or the second subset of portions. The method then transforms the set of filtered genomic portions to a reduced set of parameters by performing a principal component transformation of the set of filtered genomic portions that yields principal components for the test sample. The produced principal components can be represented in a principal component space, which contains a common principal component origin. Trisomy samples are typically grouped together and follow distinct patterns or directions, e.g., they form a vector in a two dimensional principal component space, a plane in a three dimensional principal component space, or a hyperplane in a n-dimension principal component space.

### 2) Medical Disorders and Medical Conditions

Methods described herein can be applicable to any suitable medical disorder or medical condition. Non-limiting examples of medical disorders and medical conditions include cell proliferative disorders and conditions, wasting disorders and conditions, degenerative disorders and conditions, autoimmune disorders and conditions, pre-eclampsia, chemical or environmental toxicity, liver damage or disease, kidney damage or disease, vascular disease, high blood pressure, and myocardial infarction.

In some embodiments, a cell proliferative disorder or condition sometimes is a cancer, tumor, neoplasm, metastatic disease, the like or combination thereof. A cell proliferative disorder or condition sometimes is a disorder or condition of the liver, lung, spleen, pancreas, colon, skin, bladder, eye, brain, esophagus, head, neck, ovary, testes, prostate, the like or combination thereof. Non-limiting examples of cancers include hematopoietic neoplastic disorders, which are diseases involving hyperplastic/neoplastic cells of hematopoietic origin (e.g., arising from myeloid, lymphoid or erythroid lineages, or precursor cells thereof), and can arise from poorly differentiated acute leukemias (e.g., erythroblastic leukemia and acute megakaryoblastic leukemia). Certain myeloid disorders include, but are not limited to, acute promyeloid leukemia (APML), acute myelogenous leukemia (AML) and chronic myelogenous leukemia (CML). Certain lymphoid malignancies include, but are not limited to, acute lymphoblastic leukemia (ALL), which includes B-lineage ALL and T-lineage ALL, chronic lymphocytic leukemia (CLL), prolymphocytic leukemia (PLL), hairy cell leukemia (HLL) and Waldenstrom's macroglobulinemia (WM). Certain forms of malignant lymphomas include, but are not limited to, non-Hodgkin lymphoma and variants thereof, peripheral T cell lymphomas, adult T cell leukemia/lymphoma (ATL), cutaneous T-cell lymphoma (CTCL), large granular lymphocytic leukemia (LGF), Hodgkin's disease and Reed-Sternberg disease. A cell proliferative disorder sometimes is a non-endocrine tumor or endocrine tumor. Illustrative examples of non-endocrine tumors include, but are not limited to, adenocarcinomas, acinar cell carcinomas, adenosquamous carcinomas, giant cell tumors, intraductal papillary mucinous neoplasms, mucinous cystadenocarcinomas, pancreatoblastomas, serous cystadenomas, solid and pseudopapillary tumors. An endocrine tumor sometimes is an islet cell tumor.

In some embodiments, the disclosure provides a method to determine if a test sample comprising a cancer. The method comprises providing a set of genomic portions each coupled to a copy number alteration quantification for the test sample. The genomic portions comprise portions of a reference genome to which sequence reads obtained for sample nucleic acid from the test subject have been mapped, and the copy number alteration quantification coupled to each genomic portion has been determined from a quantification of sequence reads mapped to the genomic portion. The method further comprises filtering, by a computing device, from the set of genomic portions: (i) a first subset of portions associated with copy number alterations consistently presented in a reference set of samples, and/or (ii) a second subset of portions other than representative portions derived from groups of portions identified by a clustering process applied to a reference set of samples, thereby generating a set of filtered genomic portions not containing the first subset of portions and/or the second subset of portions. The method then transforms the set of filtered genomic portions to a reduced set of parameters by performing a principal component transformation of the set of filtered genomic portions that yields principal components for the test sample. The produced principal components can be represented in a principal component space, which contains a common principal component origin. The distance between the principal components of the test sample from the common principal component origin can be determined and compared with a predetermined threshold. These tumor samples with anomalous CNA events demonstrated outlier behavior lying outside of the central cloud and thus the distance as determined above are typically greater than the predetermined threshold. In some cases, the distance is a Mahalanobis distance and the predetermined threshold greater than 300, e.g., greater than 400, greater than 450, greater than 500, or about 500 (i.e., cutoff log 10 Mahalanobis distance between 2 and 3).

In some embodiments, a wasting disorder or condition, or degenerative disorder or condition, is cirrhosis, amyotrophic lateral sclerosis (ALS), Alzheimer's disease, Parkinson's disease, multiple system atrophy, atherosclerosis, progressive supranuclear palsy, Tay-Sachs disease, diabetes, heart disease, keratoconus, inflammatory bowel disease (IBD), prostatitis, osteoarthritis, osteoporosis, rheumatoid arthritis, Huntington's disease, chronic traumatic encephalopathy, chronic obstructive pulmonary disease (COPD), tuberculosis, chronic diarrhea, acquired immune deficiency syndrome (AIDS), superior mesenteric artery syndrome, the like or combination thereof.

In some embodiments, an autoimmune disorder or condition is acute disseminated encephalomyelitis (ADEM), Addison's disease, alopecia areata, ankylosing spondylitis, antiphospholipid antibody syndrome (APS), autoimmune hemolytic anemia, autoimmune hepatitis, autoimmune inner ear disease, bullous pemphigoid, celiac disease, Chagas disease, chronic obstructive pulmonary disease, Crohns Disease (a type of idiopathic inflammatory bowel disease “IBD”), dermatomyositis, diabetes mellitus type 1, endometriosis, Goodpasture's syndrome, Graves' disease, Guillain-Barré syndrome (GBS), Hashimoto's disease, hidradenitis suppurativa, idiopathic thrombocytopenia purpura, interstitial cystitis, Lupus erythematosus, mixed connective tissue disease, morphea, multiple sclerosis (MS), myasthenia gravis, narcolepsy, euromyotonia, pemphigus vulgaris, pernicious anaemia, polymyositis, primary biliary cirrhosis, rheumatoid arthritis, schizophrenia, scleroderma, Sjögren's syndrome, temporal arteritis (also known as “giant cell arteritis”), ulcerative colitis (a type of idiopathic inflammatory bowel disease “IBD”), vasculitis, vitiligo, Wegener's granulomatosis, the like or combination thereof.

### 3) Preeclampsia

In some embodiments, the presence or absence of preeclampsia is determined by using a method or apparatus described herein. Preeclampsia is a condition in which hypertension arises in pregnancy (e.g., pregnancy-induced hypertension) and is associated with significant amounts of protein in the urine. In certain instances, preeclampsia may be associated with elevated levels of extracellular nucleic acid and/or alterations in methylation patterns. For example, a positive correlation between extracellular fetal-derived hypermethylated RASSFIA levels and the severity of pre-eclampsia has been observed. In certain instances, increased DNA methylation is observed for the H19 gene in preeclamptic placentas compared to normal controls.

### a) Pathogens

In some embodiments, the presence or absence of a pathogenic condition is determined by a method or apparatus described herein. A pathogenic condition can be caused by infection of a host by a pathogen including, but not limited to, a bacterium, virus or fungus. Since pathogens typically possess nucleic acid (e.g., genomic DNA, genomic RNA, mRNA) that can be distinguishable from host nucleic acid, methods, machines and apparatus provided herein can be used to determine the presence or absence of a pathogen. Often, pathogens possess nucleic acid with characteristics unique to a particular pathogen such as, for example, epigenetic state and/or one or more sequence variations, duplications and/or deletions. Thus, methods provided herein may be used to identify a particular pathogen or pathogen variant (e.g., strain).

### 4) Use of Cell Free Nucleic Acid

In certain instances, nucleic acid from abnormal or diseased cells associated with a particular condition or disorder is released from the cells as circulating cell-free nucleic acid (CCF-NA). For example, cancer cell nucleic acid is present in CCF-NA, and analysis of CCF-NA using methods provided herein can be used to determining whether a subject has, or is at risk of having, cancer. Analysis of the presence or absence of cancer cell nucleic acid in CCF-NA can be used for cancer screening, for example. In certain instances, levels of CCF-NA in serum can be elevated in patients with various types of cancer compared with healthy patients. Patients with metastatic diseases, for example, can sometimes have serum DNA levels approximately twice as high as non-metastatic patients. Accordingly, methods described herein can provide an outcome by processing sequencing read counts obtained from CCF-NA extracted from a sample from a subject (e.g., a subject having, suspected of having, predisposed to, or suspected as being predisposed to, a particular condition or disease).

### a) Markers

In certain instances, a polynucleotide in abnormal or diseased cells is modified with respect to nucleic acid in normal or non-diseased cells (e.g., single nucleotide alteration, single nucleotide variation, copy number alteration, copy number variation). In some instances, a polynucleotide is present in abnormal or diseased cells and not present in normal or non-diseased cells, and sometimes a polynucleotide is not present in abnormal or diseased cells and is present in normal or non-diseased cells. Thus, a marker sometimes is a single nucleotide alteration/variation and/or a copy number alteration/variation (e.g., a differentially expressed DNA or RNA (e.g., mRNA)). For example, patients with metastatic diseases may be identified by cancer-specific markers and/or certain single nucleotide polymorphisms or short tandem repeats, for example. Non-limiting examples of cancer types that may be positively correlated with elevated levels of circulating DNA include breast cancer, colorectal cancer, gastrointestinal cancer, hepatocellular cancer, lung cancer, melanoma, non-Hodgkin lymphoma, leukemia, multiple myeloma, bladder cancer, hepatoma, cervical cancer, esophageal cancer, pancreatic cancer, and prostate cancer. Various cancers can possess, and can sometimes release into the bloodstream, nucleic acids with characteristics that are distinguishable from nucleic acids from non-cancerous healthy cells, such as, for example, epigenetic state and/or sequence variations, duplications and/or deletions. Such characteristics can, for example, be specific to a particular type of cancer. Accordingly, methods described herein sometimes provide an outcome based on determining the presence or absence of a particular marker, and sometimes an outcome is presence or absence of a particular type of condition (e.g., a particular type of cancer).

### b) Types of Nucleic Acid

In some embodiments, a sample nucleic acid comprises majority nucleic acid and minority nucleic acid. In some embodiments, a majority nucleic acid comprises maternal nucleic acid and a minority nucleic acid comprises fetal nucleic acid. Accordingly, in some embodiments, a method herein comprises determining a fetal fraction. In some embodiments, a method herein comprises determining a fetal fraction for a region (e.g., a genomic region). In some embodiments, a method herein comprises determining a fetal fraction for a copy number variation region. A fetal fraction for a copy number variation region may be referred to as an affected fraction, an affected fetal fraction, and/or a fetal fraction for an affected region. As discussed above, a fetal fraction for a copy number variation region may be determined according to information (e.g., sequence information, epigenetic information) obtained for a region (e.g., a genomic region) that is identified as having a fetal copy number variation. A fetal fraction for a copy number variation region may be determined using any suitable method for quantifying fetal nucleic acid in a mixture of maternal nucleic acid and fetal nucleic acid. For example, a fetal fraction for a copy number variation region may be determined according to a sequencing-based fetal fraction (SeqFF) estimation. Methods for determining fetal fraction according to a sequencing-based fetal fraction (SeqFF) estimation are described herein and in International Patent Application Publication No. WO 2014/205401 and Kim et al. (2015) Prenatal Diagnosis 35:810-815, each of which is incorporated by reference herein. A sequencing-based fetal fraction (SeqFF) estimation may be referred to as a bin-based fetal fraction (BFF) estimation and/or a portion-specific fetal fraction estimation. In some embodiments, a fetal fraction for a copy number variation region may be determined according to allelic ratios of polymorphic sequences in fetal nucleic acid and maternal nucleic acid. Polymorphic sequences may include single nucleotide polymorphisms (SNPs), for example. Methods for determining a fetal fraction according to allelic ratios of polymorphic sequences are described herein and in U.S. Patent Application Publication No. 2011/0224087, which is incorporated by reference herein. In some embodiments, a fetal fraction for a copy number variation region may be determined according to differential epigenetic biomarkers (e.g., a quantification of differentially methylated fetal nucleic acid and maternal nucleic acid). Methods for determining fetal fraction according to a quantification of differentially methylated fetal nucleic acid and maternal nucleic acid, for example, are described herein and in U.S. Patent Application Publication No. 2010/0105049, which is incorporated by reference herein.

In some embodiments, a method herein comprises determining a fraction of minority nucleic acid in sample nucleic acid. Determining a fraction of minority nucleic acid in sample nucleic acid generally is not limited to methods that quantify a nucleic acid species based on information for a region identified as having a copy number variation, such as the methods described above. Rather, determining a fraction of minority nucleic acid in sample nucleic acid may include methods that quantify a minority nucleic acid according to information from regions across a genome and/or regions that are different than a region identified as having a copy number variation. In some embodiments, a fraction of minority nucleic acid is determined for a genomic region larger than a copy number variation region. For example, a fraction of minority nucleic acid may be determined for a genomic region that includes more genomic content (e.g., base pairs, kilobases, megabases) than the region identified as having a copy number variation. For example, for a sample where the minority nucleic acid is identified as having a trisomy of chromosome 21, a fraction of minority nucleic acid may be determined according to information (e.g., sequence information, sequence read quantifications, polymorphic sequences, differentially methylated sequences) from or in connection with a plurality of chromosomes. In this example, such a plurality of chromosomes may include all chromosomes, all autosomes, a subset of chromosomes, a subset of autosomes, a subset of chromosomes that includes chromosome 21, a subset of autosomes that includes chromosome 21, a subset of chromosomes that excludes chromosome 21, a subset of autosomes that excludes chromosome 21, or parts thereof. In some embodiments, a fraction of minority nucleic acid is determined for a genomic region that is different from the copy number variation region. For example, for a sample where the minority nucleic acid is identified as having a trisomy of chromosome 21, a fraction of minority nucleic acid may be determined according to information (e.g., sequence information, sequence read quantifications, polymorphic sequences, differentially methylated sequences) from or in connection with a chromosome other than chromosome 21.

A fraction of minority nucleic acid in sample nucleic acid may be determined using any suitable method for quantifying a species of nucleic acid in a nucleic acid mixture. For example, a fraction of minority nucleic acid may be determined according to a sequencing-based fraction estimation. Methods for determining a minority nucleic acid fraction according to a sequencing-based fraction estimation are described herein and in International Patent Application Publication No. WO 2014/205401 and Kim et al. (2015) Prenatal Diagnosis 35:810-815, each of which is incorporated by reference herein. A sequencing-based fraction estimation may be referred to as a bin-based fraction estimation and/or a portion-specific fraction estimation. In some embodiments, a fraction of minority nucleic acid may be determined according to allelic ratios of polymorphic sequences. Polymorphic sequences may include single nucleotide polymorphisms (SNPs), for example. Methods for determining a minority nucleic acid fraction according to allelic ratios of polymorphic sequences are described herein and in U.S. Patent Application Publication No. 2011/0224087, which is incorporated by reference herein. In some embodiments, a fraction of minority nucleic acid may be determined according to differential epigenetic biomarkers (e.g., a quantification of differentially methylated nucleic acid). Methods for determining a minority nucleic acid fraction according to a quantification of differentially methylated nucleic acid, for example, are described herein and in U.S. Patent Application Publication No. 2010/0105049, which is incorporated by reference herein.

In some embodiments, a minority nucleic acid comprises fetal nucleic acid. Accordingly, in some embodiments, a method herein comprises determining a fetal fraction. A fetal fraction may be determined using any suitable method for quantifying fetal nucleic acid in a mixture of maternal nucleic acid and fetal nucleic acid. For example, a fetal fraction may be determined according to a sequencing-based fetal fraction (SeqFF) estimation. Methods for determining fetal fraction according to a sequencing-based fetal fraction (SeqFF) estimation are described herein and in International Patent Application Publication No. WO 2014/205401 and Kim et al. (2015) Prenatal Diagnosis 35:810-815, each of which is incorporated by reference herein. A sequencing-based fetal fraction (SeqFF) estimation may be referred to as a bin-based fetal fraction (BFF) estimation and/or a portion-specific fetal fraction estimation. In some embodiments, a fetal fraction may be determined according to allelic ratios of polymorphic sequences in fetal nucleic acid and maternal nucleic acid. Polymorphic sequences may include single nucleotide polymorphisms (SNPs), for example. Methods for determining a fetal fraction according to allelic ratios of polymorphic sequences are described herein and in U.S. Patent Application Publication No. 2011/0224087, which is incorporated by reference herein. In some embodiments, a fetal fraction may be determined according to differential epigenetic biomarkers (e.g., a quantification of differentially methylated fetal nucleic acid and maternal nucleic acid). Methods for determining fetal fraction according to a quantification of differentially methylated fetal nucleic acid and maternal nucleic acid, for example, are described herein and in U.S. Patent Application Publication No. 2010/0105049, which is incorporated by reference herein. In some embodiments, a fetal fraction may be determined according to a chromosome Y assay. Methods for determining fetal fraction according to a chromosome Y assay are described herein and in Lo Y M, et al. (1998) Am J Hum Genet 62:768-775.

In some embodiments, a fraction for a copy number variation region and a fraction of minority nucleic acid are determined using the same methodology. For example, a fraction for a copy number variation region and a fraction of minority nucleic acid may each be determined according to a sequencing-based fraction estimation. In some embodiments, a fraction for a copy number variation region and a fraction of minority nucleic acid are determined using different methodologies. For example, a fraction for a copy number variation region may be determined according to allelic ratios of polymorphic sequences and a fraction of minority nucleic acid may be determined according to differential epigenetic biomarkers.

In some embodiments, a fetal fraction for a copy number variation region and a fetal fraction for a nucleic acid sample are determined using the same methodology. For example, a fetal fraction for a copy number variation region and a fetal fraction for a nucleic acid sample may each be determined according to a sequencing-based fetal fraction estimation. In some embodiments, a fetal fraction for a copy number variation region and a fetal fraction for a nucleic acid sample are determined using different methodologies. For example, a fetal fraction for a copy number variation region may be determined according to allelic ratios of polymorphic sequences and a fetal fraction for a nucleic acid sample may be determined according to a chromosome Y assay.

In some embodiments, a fraction for a copy number variation (e.g., a copy number variation region) is determined for a chromosome, or a part thereof. A fraction for a copy number variation determined for a chromosome, or a part thereof, refers to a quantification of a nucleic acid species based on information (e.g., sequence information, sequence read quantifications, polymorphic sequences, differentially methylated sequences) from or in connection with the chromosome, or a part thereof. In some embodiments, a fraction for a copy number variation (e.g., a copy number variation region) is determined for chromosome 13, chromosome 18, or chromosome 21. In some embodiments, a fraction of minority nucleic acid is determined for a chromosome, or part thereof that, is different than the chromosome, or part thereof, used for determining a fraction for a copy number variation. In some embodiments, a fraction of minority nucleic acid is determined for a plurality of chromosomes, or a plurality of parts of chromosomes. In some embodiments, a fraction of minority nucleic acid is determined for a plurality of autosomes, or a plurality of parts of autosomes. In some embodiments, a fraction of minority nucleic acid is determined for a plurality of regions (e.g., genomic regions). In some embodiments, a fraction of minority nucleic acid is determined for a genome-wide plurality of regions (e.g., genomic regions).

In some embodiments, a fetal fraction for a copy number variation (e.g., a copy number variation region) is determined for a chromosome, or a part thereof. A fetal fraction for a copy number variation determined for a chromosome, or a part thereof, refers to a quantification of fetal nucleic acid based on information (e.g., sequence information, sequence read quantifications, polymorphic sequences, differentially methylated sequences) from or in connection with the chromosome, or a part thereof. In some embodiments, a fetal fraction for a copy number variation (e.g., a copy number variation region) is determined for chromosome 13, chromosome 18, or chromosome 21. In some embodiments, a fetal fraction for sample nucleic acid is determined for a chromosome, or part thereof that, is different than the chromosome, or part thereof, used for determining a fetal fraction for a copy number variation. In some embodiments, a fetal fraction for sample nucleic acid is determined for a plurality of chromosomes, or a plurality of parts of chromosomes. In some embodiments, a fetal fraction for sample nucleic acid is determined for a plurality of autosomes, or a plurality of parts of autosomes. In some embodiments, a fetal fraction for sample nucleic acid is determined for a plurality of regions (e.g., genomic regions). In some embodiments, a fetal fraction for sample nucleic acid is determined for a genome-wide plurality of regions (e.g., genomic regions).

### c) Nucleic Acid Capture

In some embodiments, a sample nucleic acid (or a sample nucleic acid library) is subjected to a target capture process. Generally, a target capture process is performed by contacting sample nucleic acid (or a sample nucleic acid library) with a set of probe oligonucleotides under hybridization conditions. A set of probe oligonucleotides (e.g., capture oligonucleotides) generally includes a plurality of probe oligonucleotides having sequences that are complementary to, or substantially complementary to, sequences in sample nucleic acid. A plurality of probe oligonucleotides may include about 10 probe oligonucleotide species, about 50 probe oligonucleotide species, about 100 probe oligonucleotide species, about 500 probe oligonucleotide species, about 1,000 probe oligonucleotide species, 2,000 probe oligonucleotide species, 3,000 probe oligonucleotide species, 4,000 probe oligonucleotide species, 5000 probe oligonucleotide species, 10,000 probe oligonucleotide species, or more. Generally, a first probe oligonucleotide species has a different nucleotide sequence than a second probe oligonucleotide species, and different species of probe oligonucleotides in a set each have a different nucleotide sequence.

A probe oligonucleotide typically comprises a nucleotide sequence capable of hybridizing or annealing to a nucleic acid fragment of interest (e.g. target fragment) or a portion thereof. A probe oligonucleotide may be naturally occurring or synthetic and may be DNA or RNA based. Probe oligonucleotides can allow for specific separation of, for example, a target fragment away from other fragments in a nucleic acid sample. The term “specific” or “specificity,” as used herein, refers to the binding or hybridization of one molecule to another molecule, such as an oligonucleotide for a target polynucleotide. “Specific” or “specificity” refers to the recognition, contact, and formation of a stable complex between two molecules, as compared to substantially less recognition, contact, or complex formation of either of those two molecules with other molecules. As used herein, the terms “anneal” and “hybridize” refer to the formation of a stable complex between two molecules. The terms “probe,” probe oligonucleotide,” “capture probe,” “capture oligonucleotide,” “capture oligo,” “oligo,” or “oligonucleotide” may be used interchangeably throughout the document, when referring to probe oligonucleotides.

A probe oligonucleotide can be designed and synthesized using a suitable process, and may be of any length suitable for hybridizing to a nucleotide sequence of interest and performing separation and/or analysis processes described herein. Oligonucleotides may be designed based upon a nucleotide sequence of interest (e.g., target fragment sequence, genomic sequence, gene sequence). An oligonucleotide (e.g., a probe oligonucleotide), in some embodiments, may be about 10 to about 300 nucleotides, about 50 to about 200 nucleotides, about 75 to about 150 nucleotides, about 110 to about 130 nucleotides, or about 111, 112, 113, 114, 115, 116, 117, 118, 119, 120, 121, 122, 123, 124, 125, 126, 127, 128, or 129 nucleotides in length. An oligonucleotide may be composed of naturally occurring and/or non-naturally occurring nucleotides (e.g., labeled nucleotides), or a mixture thereof. Oligonucleotides suitable for use with embodiments described herein, may be synthesized and labeled using known techniques. Oligonucleotides may be chemically synthesized according to the solid phase phosphoramidite triester method first described by Beaucage and Caruthers (1981) Tetrahedron Letts. 22:1859-1862, using an automated synthesizer, and/or as described in Needham-VanDevanter et al. (1984) Nucleic Acids Res. 12:6159-6168. Purification of oligonucleotides can be effected by native acrylamide gel electrophoresis or by anion-exchange high-performance liquid chromatography (HPLC), for example, as described in Pearson and Regnier (1983) J. Chrom. 255:137-149.

All or a portion of a probe oligonucleotide sequence (naturally occurring or synthetic) may be substantially complementary to a target sequence or portion thereof, in some embodiments. As referred to herein, “substantially complementary” with respect to sequences refers to nucleotide sequences that will hybridize with each other. The stringency of the hybridization conditions can be altered to tolerate varying amounts of sequence mismatch. Included are target and oligonucleotide sequences that are 55% or more, 56% or more, 57% or more, 58% or more, 59% or more, 60% or more, 61% or more, 62% or more, 63% or more, 64% or more, 65% or more, 66% or more, 67% or more, 68% or more, 69% or more, 70% or more, 71% or more, 72% or more, 73% or more, 74% or more, 75% or more, 76% or more, 77% or more, 78% or more, 79% or more, 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more or 99% or more complementary to each other.

Probe oligonucleotides that are substantially complimentary to a nucleotide sequence of interest (e.g., target sequence) or portion thereof are also substantially similar to the compliment of the target sequence or relevant portion thereof (e.g., substantially similar to the anti-sense strand of the nucleic acid). One test for determining whether two nucleotide sequences are substantially similar is to determine the percent of identical nucleotide sequences shared. As referred to herein, “substantially similar” with respect to sequences refers to nucleotide sequences that are 55% or more, 56% or more, 57% or more, 58% or more, 59% or more, 60% or more, 61% or more, 62% or more, 63% or more, 64% or more, 65% or more, 66% or more, 67% or more, 68% or more, 69% or more, 70% or more, 71% or more, 72% or more, 73% or more, 74% or more, 75% or more, 76% or more, 77% or more, 78% or more, 79% or more, 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more or 99% or more identical to each other.

Hybridization conditions (e.g., annealing conditions) can be determined and/or adjusted, depending on the characteristics of the oligonucleotides used in an assay. Oligonucleotide sequence and/or length sometimes may affect hybridization to a nucleic acid sequence of interest. Depending on the degree of mismatch between an oligonucleotide and nucleic acid of interest, low, medium or high stringency conditions may be used to effect the annealing. As used herein, the term “stringent conditions” refers to conditions for hybridization and washing. Methods for hybridization reaction temperature condition optimization are known in the art, and may be found in Current Protocols in Molecular Biology, John Wiley & Sons, N.Y., 6.3.1-6.3.6 (1989). Aqueous and non-aqueous methods are described in that reference and either can be used. Non-limiting examples of stringent hybridization conditions are hybridization in 6× sodium chloride/sodium citrate (SSC) at about 45° C., followed by one or more washes in 0.2×SSC, 0.1% SDS at 50° C. Another example of stringent hybridization conditions is hybridization in 6× sodium chloride/sodium citrate (SSC) at about 45° C., followed by one or more washes in 0.2×SSC, 0.1% SDS at 55° C. A further example of stringent hybridization conditions is hybridization in 6× sodium chloride/sodium citrate (SSC) at about 45° C., followed by one or more washes in 0.2×SSC, 0.1% SDS at 60° C. Often, stringent hybridization conditions are hybridization in 6× sodium chloride/sodium citrate (SSC) at about 45° C., followed by one or more washes in 0.2×SSC, 0.1% SDS at 65° C. More often, stringency conditions are 0.5M sodium phosphate, 7% SDS at 65° C., followed by one or more washes at 0.2×SSC, 1% SDS at 65° C. Stringent hybridization temperatures can also be altered (i.e. lowered) with the addition of certain organic solvents, formamide for example. Organic solvents, like formamide, reduce the thermal stability of double-stranded polynucleotides, so that hybridization can be performed at lower temperatures, while still maintaining stringent conditions and extending the useful life of nucleic acids that may be heat labile.

In some embodiments, one or more probe oligonucleotides are associated with an affinity ligand such as a member of a binding pair (e.g., biotin) or antigen that can bind to a capture agent such as avidin, streptavidin, an antibody, or a receptor. For example, a probe oligonucleotide may be biotinylated such that it can be captured onto a streptavidin-coated bead.

In some embodiments, one or more probe oligonucleotides and/or capture agents are effectively linked to a solid support or substrate. A solid support or substrate can be any physically separable solid to which a probe oligonucleotide can be directly or indirectly attached including, but not limited to, surfaces provided by microarrays and wells, and particles such as beads (e.g., paramagnetic beads, magnetic beads, microbeads, nanobeads), microparticles, and nanoparticles. Solid supports also can include, for example, chips, columns, optical fibers, wipes, filters (e.g., flat surface filters), one or more capillaries, glass and modified or functionalized glass (e.g., controlled-pore glass (CPG)), quartz, mica, diazotized membranes (paper or nylon), polyformaldehyde, cellulose, cellulose acetate, paper, ceramics, metals, metalloids, semiconductive materials, quantum dots, coated beads or particles, other chromatographic materials, magnetic particles; plastics (including acrylics, polystyrene, copolymers of styrene or other materials, polybutylene, polyurethanes, TEFLON™, polyethylene, polypropylene, polyamide, polyester, polyvinylidenedifluoride (PVDF), and the like), polysaccharides, nylon or nitrocellulose, resins, silica or silica-based materials including silicon, silica gel, and modified silicon, Sephadex®, Sepharose®, carbon, metals (e.g., steel, gold, silver, aluminum, silicon and copper), inorganic glasses, conducting polymers (including polymers such as polypyrole and polyindole); micro or nanostructured surfaces such as nucleic acid tiling arrays, nanotube, nanowire, or nanoparticulate decorated surfaces; or porous surfaces or gels such as methacrylates, acrylamides, sugar polymers, cellulose, silicates, or other fibrous or stranded polymers. In some embodiments, the solid support or substrate may be coated using passive or chemically-derivatized coatings with any number of materials, including polymers, such as dextrans, acrylamides, gelatins or agarose. Beads and/or particles may be free or in connection with one another (e.g., sintered). In some embodiments, the solid phase can be a collection of particles. In some embodiments, the particles can comprise silica, and the silica may comprise silica dioxide. In some embodiments the silica can be porous, and in certain embodiments the silica can be non-porous. In some embodiments, the particles further comprise an agent that confers a paramagnetic property to the particles. In certain embodiments, the agent comprises a metal, and in certain embodiments the agent is a metal oxide, (e.g., iron or iron oxides, where the iron oxide contains a mixture of Fe2+ and Fe3+). The probe oligonucleotides may be linked to the solid support by covalent bonds or by non-covalent interactions and may be linked to the solid support directly or indirectly (e.g., via an intermediary agent such as a spacer molecule or biotin). A probe oligonucleotide may be linked to the solid support before, during or after nucleic acid capture.

Nucleic acid that has been modified, such as modified by the addition of adapter sequences described herein, may be captured. In some embodiments, unmodified nucleic acid is captured. Nucleic acid may be amplified before and/or after capture, in some embodiments, by an amplification process such as PCR. The term “captured nucleic acid” generally includes nucleic acid that has been captured and includes nucleic acid that has been captured and amplified. Captured nucleic acid may be subjected to additional rounds of capture and amplification, in some embodiments. Captured nucleic acid may be sequenced, such as by a sequencing process described herein.

### 5) Mosaicism Ratio (MR) and Using MR to Detect Genetic Variation

A mosaicism ratio (a metric obtainable from prenatal cfDNA testing) may be used to identify patients in which genetic variations may exist in a mosaic form. In some instances, a majority of cells may develop from a zygote into placental trophoblast/chorionic ectoderm, while a small minority of cells develop into chorionic villi/mesoderm, and only a number of cells (e.g., 2) go on to form the embryo and amniotic tissues. When an error in cell division occurs at differing levels in this chain it can lead to differing levels of fetal or placental (or both) mosaicism which can have radically different clinical implications. When this is the case, not all cell free trophoblastic DNA in maternal plasma is affected. This observation can be used to calculate a mosaicism ratio (MR) of affected cell-free DNA and total cell-free DNA.

In various embodiments, the MR is calculated by: (a) determining a fraction of nucleic acid having a genetic variation in circulating cell free nucleic acid, (b) determining a fraction of fetal nucleic acid in the circulating cell free nucleic acid, and (c) generating the MR using the fraction of (a) to the fraction of (b). Furthermore, it has been discovered that the MR can be used to identify patients with a higher chance for discordant positive results due to a mosaicism (e.g., confined placental mosaicism, or CPM). For example, the MR may be used classify a presence or absence of genetic mosaicism for a genetic variation region. In certain embodiments, the presence of genetic mosaicism is classified for a genetic variation region when the value of the MR is between a range (e.g., about 0.2 to about 0.7). In certain embodiments, the absence of genetic mosaicism is classified for a genetic variation region when the value of the MR is greater than a pre-determined value (e.g., 0.7). The use of the mosaicism ratio in such circumstances has many advantages over conventional processes for confirming positive NIPT results including a non-invasive approach to confirm the positive NIPT results.

Furthermore, the knowledge of whether a mosaicism is present or absent may be used by physicians and genetic counselors to better interpret positive NIPT results, which may lead to improved posttest counseling and overall prenatal care. For example, a presence of genetic mosaicism classification (e.g., an MR of 20%-70%) for a genetic variation region may be interpreted as non-standard positive NIPT result with a mosaic comment. An absence of genetic mosaicism classification for a genetic variation region (e.g., an MR greater than 70%) may be interpreted as a standard positive NIPT result (e.g., a positive result for a fetal genetic variation), an affected fetus, a fetal genetic variation, a full genetic variation, a true genetic variation, a complete genetic variation, and the like. No classification may be provided when the value of the MR is below a certain threshold (e.g., an MR less than 20%) for a genetic variation region, which may be interpreted as a negative NIPT result for a fetal genetic variation. In some instances, a disproportionally high MR (in consideration with other sequencing metrics, such as a considerably elevated z-score) may suggest that the sample associated with the MR may likely contain a maternal genetic event or mosaicism.

Mosaicism ratio and similar laboratory metrics may also be used in other fields beside prenatal cfDNA testing or NIPT, for example, in the field of organ transplantation and/or cancer diagnosis/treatment. The fraction of fetal nucleic acid in the MR may be substituted by the fraction of donor nucleic acid or tumor nucleic acid.

### a) Determining a Fraction of Nucleic Acid Having a Genetic Variation

In some embodiments, a method herein comprises determining a fraction of nucleic acid having a genetic variation in circulating cell free nucleic acid. Determining a fraction of nucleic acid refers to quantifying a particular species of nucleic acid in a nucleic acid mixture. For example, determining a fraction of nucleic acid may refer to quantifying a minority nucleic acid species, quantifying fetal nucleic acid, quantifying cancer nucleic acid, and the like. Determining a fraction of nucleic acid having a genetic variation refers to quantifying a subset of nucleic acid (e.g., a subset of nucleic acid fragments, a subset of sequence reads) for which a genetic variation is identified. In some embodiments, determining a fraction of nucleic acid having a genetic variation refers to quantifying a subset of nucleic acid (e.g., a subset of nucleic acid fragments, a subset of sequence reads) from a region (e.g., a genomic region) for which a genetic variation is identified. In some embodiments, determining a fraction of nucleic acid having a genetic variation refers to quantifying a subset of nucleic acid for a species (e.g., a subset of nucleic acid fragments for a species, a subset of sequence reads for a species) from a region (e.g., a genomic region) for which a genetic variation is identified. For example, for a sample comprising maternal nucleic acid and fetal nucleic acid, where the fetal nucleic acid is identified as having a trisomy of chromosome 21, determining a fraction of nucleic acid having a genetic variation refers to determining a fetal fraction based on information (e.g., sequence information, sequencing data, sequence read quantifications, polymorphic sequences, differentially methylated sequences) from or in connection with chromosome 21, or a part thereof.

In some embodiments, a method herein comprises determining a fraction for a region (e.g., a genomic region). In some embodiments, a method herein comprises determining a fraction for a genetic variation region. A fraction for a genetic variation region may be referred to as an affected fraction or a fraction for an affected region. As discussed above, a fraction for a genetic variation region may be determined according to information (e.g., sequence information, epigenetic information) obtained for a region (e.g., a genomic region) that is identified as having a genetic variation. A fraction for a genetic variation region may be determined using any suitable method for quantifying a species of nucleic acid in a nucleic acid mixture. For example, a fraction for a genetic variation region may be determined according to a sequencing-based fraction estimation. Methods for determining a nucleic acid fraction according to a sequencing-based fraction estimation are described herein and in International Patent Application Publication No. WO 2014/205401 and Kim et al. (2015) Prenatal Diagnosis 35:810-815, each of which is incorporated in its entirety by reference herein for all purposes. A sequencing-based fraction estimation may be referred to as a bin-based fraction estimation and/or a portion-specific fraction estimation. In some embodiments, a fraction for a genetic variation region may be determined according to allelic ratios of polymorphic sequences. Polymorphic sequences may include single nucleotide polymorphisms (SNPs), for example. Methods for determining a nucleic acid fraction according to allelic ratios of polymorphic sequences are described herein and in U.S. Patent Application Publication No. 2011/0224087, which is incorporated in its entirety by reference herein for all purposes. In some embodiments, a fraction for a genetic variation region may be determined according to differential epigenetic biomarkers (e.g., a quantification of differentially methylated nucleic acid). Methods for determining a nucleic acid fraction according to a quantification of differentially methylated nucleic acid, for example, are described herein and in U.S. Patent Application Publication No. 2010/0105049, which is incorporated in its entirety by reference herein for all purposes.

In some embodiments, the circulating cell free nucleic acid comprises majority nucleic acid and minority nucleic acid. In some embodiments, a majority nucleic acid comprises maternal nucleic acid and a minority nucleic acid comprises fetal nucleic acid. Accordingly, in some embodiments, a method herein comprises determining a fetal fraction. In some embodiments, a method herein comprises determining a fetal fraction for a region (e.g., a genomic region). In some embodiments, a method herein comprises determining a fetal fraction for a genetic variation region. A fetal fraction for a genetic variation region may be referred to as an affected fraction, an affected fetal fraction, and/or a fetal fraction for an affected region. As discussed above, a fetal fraction for a genetic variation region may be determined according to information (e.g., sequence information, epigenetic information) obtained for a region (e.g., a genomic region) that is identified as having a fetal genetic variation. A fetal fraction for a genetic variation region may be determined using any suitable method for quantifying fetal nucleic acid in a mixture of maternal nucleic acid and fetal nucleic acid. For example, a fetal fraction for a genetic variation region may be determined according to a sequencing-based fetal fraction (SeqFF) estimation. Methods for determining fetal fraction according to a sequencing-based fetal fraction (SeqFF) estimation are described herein and in International Patent Application Publication No. WO 2014/205401 and Kim et al. (2015) Prenatal Diagnosis 35:810-815, each of which is incorporated in its entirety by reference herein for all purposes. A sequencing-based fetal fraction (SeqFF) estimation may be referred to as a bin-based fetal fraction (BFF) estimation and/or a portion-specific fetal fraction estimation. In some embodiments, a fetal fraction for a genetic variation region may be determined according to allelic ratios of polymorphic sequences in fetal nucleic acid and maternal nucleic acid. Polymorphic sequences may include single nucleotide polymorphisms (SNPs), for example. Methods for determining a fetal fraction according to allelic ratios of polymorphic sequences are described herein and in U.S. Patent Application Publication No. 2011/0224087, which is incorporated in its entirety by reference herein for all purposes. In some embodiments, a fetal fraction for a genetic variation region may be determined according to differential epigenetic biomarkers (e.g., a quantification of differentially methylated fetal nucleic acid and maternal nucleic acid). Methods for determining fetal fraction according to a quantification of differentially methylated fetal nucleic acid and maternal nucleic acid, for example, are described herein and in U.S. Patent Application Publication No. 2010/0105049, which is incorporated in its entirety by reference herein for all purposes.

### b) Determining a Fraction of Fetal/Minority Nucleic Acid

In some embodiments, a method herein comprises determining a fraction of minority (e.g., fetal) nucleic acid in circulating cell free nucleic acid. Determining a fraction of minority nucleic acid in sample nucleic acid generally is not limited to methods that quantify a nucleic acid species based on information for a region identified as having a genetic variation, such as the methods described above. Rather, determining a fraction of minority nucleic acid in sample nucleic acid may include methods that quantify a minority nucleic acid according to information from regions across a genome and/or regions that are different than a region identified as having a genetic variation. In some embodiments, a fraction of minority nucleic acid is determined for a genomic region larger than a genetic variation region. For example, a fraction of minority nucleic acid may be determined for a genomic region that includes more genomic content (e.g., base pairs, kilobases, megabases) than the region identified as having a genetic variation. For example, for a sample where the minority nucleic acid is identified as having a trisomy of chromosome 21, a fraction of minority nucleic acid may be determined according to information (e.g., sequence information, sequence read quantifications, polymorphic sequences, differentially methylated sequences) from or in connection with a plurality of chromosomes. In this example, such a plurality of chromosomes may include all chromosomes, all autosomes, a subset of chromosomes, a subset of autosomes, a subset of chromosomes that includes chromosome 21, a subset of autosomes that includes chromosome 21, a subset of chromosomes that excludes chromosome 21, a subset of autosomes that excludes chromosome 21, or parts thereof. In some embodiments, a fraction of minority nucleic acid is determined for a genomic region that is different from the genetic variation region. For example, for a sample where the minority nucleic acid is identified as having a trisomy of chromosome 21, a fraction of minority nucleic acid may be determined according to information (e.g., sequence information, sequence read quantifications, polymorphic sequences, differentially methylated sequences) from or in connection with a chromosome other than chromosome 21.

A fraction of minority nucleic acid in sample nucleic acid may be determined using any suitable method for quantifying a species of nucleic acid in a nucleic acid mixture. For example, a fraction of minority nucleic acid may be determined according to a sequencing-based fraction estimation. Methods for determining a minority nucleic acid fraction according to a sequencing-based fraction estimation are described herein and in International Patent Application Publication No. WO 2014/205401 and Kim et al. (2015) Prenatal Diagnosis 35:810-815, each of which is incorporated in its entirety by reference herein for all purposes. A sequencing-based fraction estimation may be referred to as a bin-based fraction estimation and/or a portion-specific fraction estimation. In some embodiments, a fraction of minority nucleic acid may be determined according to allelic ratios of polymorphic sequences. Polymorphic sequences may include single nucleotide polymorphisms (SNPs), for example. Methods for determining a minority nucleic acid fraction according to allelic ratios of polymorphic sequences are described herein and in U.S. Patent Application Publication No. 2011/0224087, which is incorporated in its entirety by reference herein for all purposes. In some embodiments, a fraction of minority nucleic acid may be determined according to differential epigenetic biomarkers (e.g., a quantification of differentially methylated nucleic acid). Methods for determining a minority nucleic acid fraction according to a quantification of differentially methylated nucleic acid, for example, are described herein and in U.S. Patent Application Publication No. 2010/0105049, which is incorporated in its entirety by reference herein for all purposes.

In some embodiments, a minority nucleic acid comprises fetal nucleic acid. Accordingly, in some embodiments, a method herein comprises determining a fetal fraction. A fetal fraction may be determined using any suitable method for quantifying fetal nucleic acid in a mixture of maternal nucleic acid and fetal nucleic acid. For example, a fetal fraction may be determined according to a sequencing-based fetal fraction (SeqFF) estimation. Methods for determining fetal fraction according to a sequencing-based fetal fraction (SeqFF) estimation are described herein and in International Patent Application Publication No. WO 2014/205401 and Kim et al. (2015) Prenatal Diagnosis 35:810-815, each of which is incorporated in its entirety by reference herein for all purposes. A sequencing-based fetal fraction (SeqFF) estimation may be referred to as a bin-based fetal fraction (BFF) estimation and/or a portion-specific fetal fraction estimation. In some embodiments, a fetal fraction may be determined according to allelic ratios of polymorphic sequences in fetal nucleic acid and maternal nucleic acid. Polymorphic sequences may include single nucleotide polymorphisms (SNPs), for example. Methods for determining a fetal fraction according to allelic ratios of polymorphic sequences are described herein and in U.S. Patent Application Publication No. 2011/0224087, which is incorporated in its entirety by reference herein for all purposes. In some embodiments, a fetal fraction may be determined according to differential epigenetic biomarkers (e.g., a quantification of differentially methylated fetal nucleic acid and maternal nucleic acid). Methods for determining fetal fraction according to a quantification of differentially methylated fetal nucleic acid and maternal nucleic acid, for example, are described herein and in U.S. Patent Application Publication No. 2010/0105049, which is incorporated in its entirety by reference herein for all purposes. In some embodiments, a fetal fraction may be determined according to a chromosome Y assay. Methods for determining fetal fraction according to a chromosome Y assay are described herein and in Lo Y M, et al. (1998) Am J Hum Genet 62:768-775.

In some embodiments, a fraction for a genetic variation region and a fraction of minority nucleic acid are determined using the same methodology. For example, a fraction for a genetic variation region and a fraction of minority nucleic acid may each be determined according to a sequencing-based fraction estimation. In some embodiments, a fraction for a genetic variation region and a fraction of minority nucleic acid are determined using different methodologies. For example, a fraction for a genetic variation region may be determined according to allelic ratios of polymorphic sequences and a fraction of minority nucleic acid may be determined according to differential epigenetic biomarkers.

In some embodiments, a fetal fraction for a genetic variation region and a fetal fraction for a nucleic acid sample are determined using the same methodology. For example, a fetal fraction for a genetic variation region and a fetal fraction for a nucleic acid sample may each be determined according to a sequencing-based fetal fraction estimation. In some embodiments, a fetal fraction for a genetic variation region and a fetal fraction for a nucleic acid sample are determined using different methodologies. For example, a fetal fraction for a genetic variation region may be determined according to allelic ratios of polymorphic sequences and a fetal fraction for a nucleic acid sample may be determined according to a chromosome Y assay.

In some embodiments, a fraction for a genetic variation (e.g., a genetic variation region) is determined for a chromosome, or a part thereof. A fraction for a genetic variation determined for a chromosome, or a part thereof, refers to a quantification of a nucleic acid species based on information (e.g., sequence information, sequence read quantifications, polymorphic sequences, differentially methylated sequences) from or in connection with the chromosome, or a part thereof. In some embodiments, a fraction for a genetic variation (e.g., a genetic variation region) is determined for chromosome 13, chromosome 18, or chromosome 21. In some embodiments, a fraction of minority nucleic acid is determined for a chromosome, or part thereof that, is different than the chromosome, or part thereof, used for determining a fraction for a genetic variation. In some embodiments, a fraction of minority nucleic acid is determined for a plurality of chromosomes, or a plurality of parts of chromosomes. In some embodiments, a fraction of minority nucleic acid is determined for a plurality of autosomes, or a plurality of parts of autosomes. In some embodiments, a fraction of minority nucleic acid is determined for a plurality of regions (e.g., genomic regions). In some embodiments, a fraction of minority nucleic acid is determined for a genome-wide plurality of regions (e.g., genomic regions).

In some embodiments, a fetal fraction for a genetic variation (e.g., a genetic variation region) is determined for a chromosome, or a part thereof. A fetal fraction for a genetic variation determined for a chromosome, or a part thereof, refers to a quantification of fetal nucleic acid based on information (e.g., sequence information, sequence read quantifications, polymorphic sequences, differentially methylated sequences) from or in connection with the chromosome, or a part thereof. In some embodiments, a fetal fraction for a genetic variation (e.g., a genetic variation region) is determined for chromosome 13, chromosome 18, or chromosome 21. In some embodiments, a fetal fraction for sample nucleic acid is determined for a chromosome, or part thereof that, is different than the chromosome, or part thereof, used for determining a fetal fraction for a genetic variation. In some embodiments, a fetal fraction for sample nucleic acid is determined for a plurality of chromosomes, or a plurality of parts of chromosomes. In some embodiments, a fetal fraction for sample nucleic acid is determined for a plurality of autosomes, or a plurality of parts of autosomes. In some embodiments, a fetal fraction for sample nucleic acid is determined for a plurality of regions (e.g., genomic regions). In some embodiments, a fetal fraction for sample nucleic acid is determined for a genome-wide plurality of regions (e.g., genomic regions).

### c) Generating an MR as a Fraction for a Genetic Variation to a Fraction of Minority/Fetal Nucleic Acid

In some embodiments, a method herein comprises generating an MR using a fraction for a genetic variation to a fraction of minority nucleic acid. In some embodiments, the generating comprises comparing a fraction for a genetic variation to a fraction of minority nucleic acid to generate a ratio. For example, a ratio may be a fraction for a genetic variation divided by a fraction of minority nucleic acid.

In some embodiments, a method herein comprises comparing a fetal fraction for a genetic variation to a fetal fraction for sample nucleic acid. In some embodiments, comparing a fetal fraction for a genetic variation to a fetal fraction for sample nucleic acid comprises generating a ratio. For example, a ratio may be a fetal fraction for a genetic variation divided by a fetal fraction for sample nucleic acid.

### C. NIPT PIPELINE

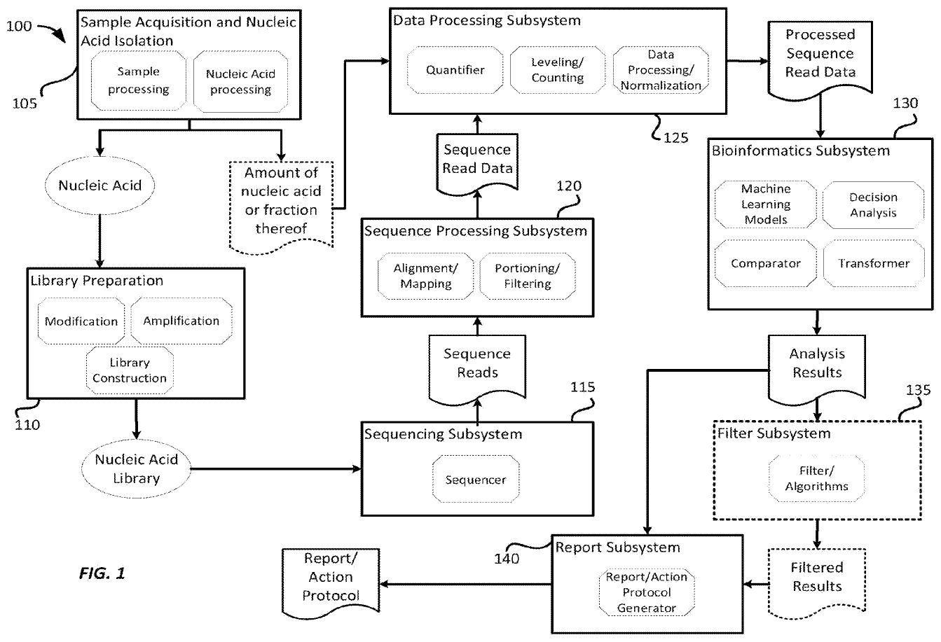

FIG. 1 is a block diagram of a clinical test pipeline 100 that implements a NIPT procedure or assay such as MaterniTR GENOME for analyzing nucleic acids in accordance with various embodiments. In some embodiments, nucleic acid fragments in a mixture of nucleic acid fragments are analyzed. Nucleic acid fragments may be referred to as nucleic acid templates, and the terms may be used interchangeably herein. A mixture of nucleic acids can comprise two or more nucleic acid fragment species having the same or different nucleotide sequences, different fragment lengths, different origins (e.g., genomic origins, fetal vs. maternal origins, cell or tissue origins, cancer vs. non-cancer origin, tumor vs. non-tumor origin, sample origins, subject origins, and the like), or combinations thereof.

The terms “nucleic acid,” “nucleic acid molecule,” “nucleic acid fragment,” and “nucleic acid template” may be used interchangeably throughout the disclosure. The terms refer to nucleic acids of any composition from, such as DNA (e.g., complementary DNA (cDNA), genomic DNA (gDNA) and the like), RNA (e.g., message RNA (mRNA), short inhibitory RNA (siRNA), ribosomal RNA (rRNA), tRNA, microRNA, RNA highly expressed by a fetus or placenta, and the like), and/or DNA or RNA analogs (e.g., containing base analogs, sugar analogs and/or a non-native backbone and the like), RNA/DNA hybrids and polyamide nucleic acids (PNAs), all of which can be in single- or double-stranded form, and unless otherwise limited, can encompass known analogs of natural nucleotides that can function in a similar manner as naturally occurring nucleotides. A nucleic acid may be, or may be from, a plasmid, phage, virus, bacterium, autonomously replicating sequence (ARS), mitochondria, centromere, artificial chromosome, chromosome, or other nucleic acid able to replicate or be replicated in vitro or in a host cell, a cell, a cell nucleus or cytoplasm of a cell in certain embodiments. A template nucleic acid in some embodiments can be from a single chromosome (e.g., a nucleic acid sample may be from one chromosome of a sample obtained from a diploid organism). Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, single nucleotide polymorphisms (SNPs), and complementary sequences as well as the sequence explicitly indicated. Specifically, degenerate codon substitutions may be achieved by generating sequences in which the third position of one or more selected (or all) codons is substituted with mixed-base and/or deoxyinosine residues. The term nucleic acid is used interchangeably with locus, gene, cDNA, and mRNA encoded by a gene. The term also may include, as equivalents, derivatives, variants and analogs of RNA or DNA synthesized from nucleotide analogs, single-stranded (“sense” or “antisense,” “plus” strand or “minus” strand, “forward” reading frame or “reverse” reading frame) and double-stranded polynucleotides. The term “gene” refers to a section of DNA involved in producing a polypeptide chain; and generally, includes regions preceding and following the coding region (leader and trailer) involved in the transcription/translation of the gene product and the regulation of the transcription/translation, as well as intervening sequences (introns) between individual coding regions (exons). A nucleotide or base generally refers to the purine and pyrimidine molecular units of nucleic acid (e.g., adenine (A), thymine (T), guanine (G), and cytosine (C)). For RNA, the base thymine is replaced with uracil. Nucleic acid length or size may be expressed as a number of bases.

At block 105, nucleic acid or a nucleic acid mixture utilized in systems, methods and products described herein may be isolated from a sample obtained from a subject (e.g., a test subject). In some embodiments, nucleic acid is extracted from cells using a cell lysis procedure. Cell lysis procedures and reagents are known in the art and may generally be performed by chemical (e.g., detergent, hypotonic solutions, enzymatic procedures, and the like, or combination thereof), physical (e.g., French press, sonication, and the like), or electrolytic lysis methods. Any suitable lysis procedure can be utilized. For example, chemical methods generally employ lysing agents to disrupt cells and extract the nucleic acids from the cells, followed by treatment with chaotropic salts. Physical methods such as freeze/thaw followed by grinding, the use of cell presses and the like also are useful. In some instances, a high salt and/or an alkaline lysis procedure may be utilized.

In some embodiments, nucleic acids can include extracellular nucleic acid. The term “extracellular nucleic acid” as used herein can refer to nucleic acid isolated from a source having substantially no cells and also is referred to as “cell-free” nucleic acid, “circulating cell-free nucleic acid” (e.g., CCF fragments, ccf DNA), “circulating tumor DNA” (ctDNA), “cell-free RNA” or “extracellular RNA” (exRNA), and/or “cell-free circulating nucleic acid.” Extracellular nucleic acid can be present in and obtained from blood (e.g., from the blood of a human subject). Extracellular nucleic acid often includes no detectable cells and may contain cellular elements or cellular remnants. Non-limiting examples of acellular sources for extracellular nucleic acid are blood, blood plasma, blood serum and urine. As used herein, the term “obtain cell-free circulating sample nucleic acid” includes obtaining a sample directly (e.g., collecting a sample, e.g., a test sample) or obtaining a sample from another who has collected a sample. Without being limited by theory, extracellular nucleic acid may be a product of cell apoptosis and cell breakdown, which provides basis for extracellular nucleic acid often having a series of lengths across a spectrum (e.g., a “ladder”). In some embodiments, sample nucleic acid from a test subject is circulating cell-free nucleic acid. In some embodiments, circulating cell free nucleic acid is from blood plasma or blood serum from a test subject.

A subject can be any living or non-living organism, including but not limited to a human, a non-human animal, a plant, a bacterium, a fungus, a protest or a pathogen. Any human or non-human animal can be selected, and may include, for example, mammal, reptile, avian, amphibian, fish, ungulate, ruminant, bovine (e.g., cattle), equine (e.g., horse), caprine and ovine (e.g., sheep, goat), swine (e.g., pig), camelid (e.g., camel, llama, alpaca), monkey, ape (e.g., gorilla, chimpanzee), ursid (e.g., bear), poultry, dog, cat, mouse, rat, fish, dolphin, whale and shark. A subject may be a male or female (e.g., woman, a pregnant woman). A subject may be any age (e.g., an embryo, a fetus, an infant, a child, an adult). A subject may be a cancer patient, a patient suspected of having cancer, a patient in remission, a patient with a family history of cancer, and/or a subject obtaining a cancer screen.

Nucleic acid may be isolated from any type of suitable biological specimen or sample (e.g., a test sample). A sample or test sample can be any specimen that is isolated or obtained from a subject or part thereof (e.g., a human subject, a pregnant female, a cancer patient, a fetus, a tumor). A sample sometimes is from a pregnant female subject bearing a fetus at any stage of gestation (e.g., first, second or third trimester for a human subject), and sometimes is from a post-natal subject. A sample sometimes is from a pregnant subject bearing a fetus that is euploid for all chromosomes, and sometimes is from a pregnant subject bearing a fetus having a chromosome aneuploidy (e.g., one, three (i.e., trisomy (e.g., T21, T18, T13)), or four copies of a chromosome) or other genetic variation. Non-limiting examples of specimens include fluid or tissue from a subject, including, without limitation, blood or a blood product (e.g., serum, plasma, or the like), umbilical cord blood, chorionic villi, amniotic fluid, cerebrospinal fluid, spinal fluid, lavage fluid (e.g., bronchoalveolar, gastric, peritoneal, ductal, ear, arthroscopic), biopsy sample (e.g., from pre-implantation embryo; cancer biopsy), celocentesis sample, cells (blood cells, placental cells, embryo or fetal cells, fetal nucleated cells or fetal cellular remnants, normal cells, abnormal cells (e.g., cancer cells)) or parts thereof (e.g., mitochondrial, nucleus, extracts, or the like), washings of female reproductive tract, urine, feces, sputum, saliva, nasal mucous, prostate fluid, lavage, semen, lymphatic fluid, bile, tears, sweat, breast milk, breast fluid, the like or combinations thereof. In some embodiments, a biological sample is a cervical swab from a subject. A fluid or tissue sample from which nucleic acid is extracted may be acellular (e.g., cell-free). In some embodiments, a fluid or tissue sample may contain cellular elements or cellular remnants. In some embodiments, fetal cells or cancer cells may be included in the sample.

A sample can be a liquid sample. A liquid sample can comprise extracellular nucleic acid (e.g., circulating cell-free DNA). Non-limiting examples of liquid samples, include, blood or a blood product (e.g., serum, plasma, or the like), urine, biopsy sample (e.g., liquid biopsy for the detection of cancer), a liquid sample described above, the like or combinations thereof. In certain embodiments, a sample is a liquid biopsy, which generally refers to an assessment of a liquid sample from a subject for the presence, absence, progression or remission of a disease (e.g., cancer). A liquid biopsy can be used in conjunction with, or as an alternative to, a sold biopsy (e.g., tumor biopsy). In certain instances, extracellular nucleic acid is analyzed in a liquid biopsy.

In some embodiments, a biological sample may be blood, plasma or serum. The term “blood” encompasses whole blood, blood product or any fraction of blood, such as serum, plasma, buffy coat, or the like as conventionally defined. Blood or fractions thereof often comprise nucleosomes. Nucleosomes comprise nucleic acids and are sometimes cell-free or intracellular. Blood also comprises buffy coats. Buffy coats are sometimes isolated by utilizing a ficoll gradient. Buffy coats can comprise white blood cells (e.g., leukocytes, T-cells, B-cells, platelets, and the like). Blood plasma refers to the fraction of whole blood resulting from centrifugation of blood treated with anticoagulants. Blood serum refers to the watery portion of fluid remaining after a blood sample has coagulated. Fluid or tissue samples often are collected in accordance with standard protocols hospitals or clinics generally follow. For blood, an appropriate amount of peripheral blood (e.g., between 3 to 40 milliliters, between 5 to 50 milliliters) often is collected and can be stored according to standard procedures prior to or after preparation.

An analysis of nucleic acid found in a subject's blood may be performed using, e.g., whole blood, serum, or plasma. An analysis of fetal DNA found in maternal blood, for example, may be performed using, e.g., whole blood, serum, or plasma. An analysis of tumor DNA found in a patient's blood, for example, may be performed using, e.g., whole blood, serum, or plasma. Methods for preparing serum or plasma from blood obtained from a subject (e.g., a maternal subject; cancer patient) are known. For example, a subject's blood (e.g., a pregnant woman's blood; cancer patient's blood) can be placed in a tube containing EDTA or a specialized commercial product such as Vacutainer SST (Becton Dickinson, Franklin Lakes, N.J.) to prevent blood clotting, and plasma can then be obtained from whole blood through centrifugation. Serum may be obtained with or without centrifugation-following blood clotting. If centrifugation is used then it is typically, though not exclusively, conducted at an appropriate speed, e.g., 1,500-3,000 times g. Plasma or serum may be subjected to additional centrifugation steps before being transferred to a fresh tube for nucleic acid extraction. In addition to the acellular portion of the whole blood, nucleic acid may also be recovered from the cellular fraction, enriched in the buffy coat portion, which can be obtained following centrifugation of a whole blood sample from the subject and removal of the plasma.

In some embodiments, the nucleic acid or the nucleic acid mixture (e.g., extracellular nucleic acid) is enriched or relatively enriched for a subpopulation or species of nucleic acid. Nucleic acid subpopulations can include, for example, fetal nucleic acid, maternal nucleic acid, cancer nucleic acid, patient nucleic acid, nucleic acid comprising fragments of a particular length or range of lengths, or nucleic acid from a particular genome region (e.g., single chromosome, set of chromosomes, and/or certain chromosome regions). Such enriched samples can be used in conjunction with a method provided herein. Thus, in certain embodiments, methods of the technology comprise an additional step of enriching for a subpopulation of nucleic acid in a sample, such as, for example, cancer or fetal nucleic acid. In certain embodiments, a method for determining fraction of cancer cell nucleic acid or fetal fraction also can be used to enrich for cancer or fetal nucleic acid. In certain embodiments, nucleic acid from normal tissue (e.g., non-cancer cells) is selectively removed (partially, substantially, almost completely or completely) from the sample. In certain embodiments, maternal nucleic acid is selectively removed (partially, substantially, almost completely or completely) from the sample. In certain embodiments, enriching for a particular low copy number species nucleic acid (e.g., cancer or fetal nucleic acid) may improve quantitative sensitivity. Methods for enriching a sample for a particular species of nucleic acid are described, for example, in U.S. Pat. No. 6,927,028, International Patent Application Publication No. WO 2007/140417, International Patent Application Publication No. WO 2007/147063, International Patent Application Publication No. WO 2009/032779, International Patent Application Publication No. WO 2009/032781, International Patent Application Publication No. WO 2010/033639, International Patent Application Publication No. WO 2011/034631, International Patent Application Publication No. WO 2006/056480, and International Patent Application Publication No. WO 2011/143659, the entire content of each is incorporated herein by reference for all purposes, including all text, tables, equations and drawings, for all purposes.

The amount of nucleic acid (e.g., concentration, relative amount, absolute amount, copy number, and the like) in a sample may be determined. The amount of a minority nucleic acid (e.g., concentration, relative amount, absolute amount, copy number, and the like) in nucleic acid is determined in some embodiments. In certain embodiments, the amount of a minority nucleic acid species in a sample is referred to as “minority species fraction.” In some embodiments “minority species fraction” refers to the fraction of a minority nucleic acid species in circulating cell-free nucleic acid in a sample (e.g., a blood sample, a serum sample, a plasma sample, a urine sample) obtained from a subject. The amount of a minority nucleic acid in extracellular nucleic acid can be quantified and used in conjunction with a method provided herein. Thus, in certain embodiments, methods described herein comprise an additional step of determining the amount of a minority nucleic acid. The amount of a minority nucleic acid can be determined in a sample from a subject before or after processing to prepare sample nucleic acid. In certain embodiments, the amount of a minority nucleic acid is determined in a sample after sample nucleic acid is processed and prepared, which amount is utilized for further assessment. In some embodiments, an outcome comprises factoring the minority species fraction in the sample nucleic acid (e.g., adjusting counts, removing samples, making a call or not making a call).

A determination of minority species fraction can be performed before, during, or at any one point in clinical test pipeline 100 described herein, or after certain processes described herein (e.g., detection of a genetic variation or genetic alteration). For example, to conduct a genetic variation/genetic alteration determination method with a certain sensitivity or specificity, a minority nucleic acid quantification method may be implemented prior to, during or after genetic variation/genetic alteration determination to identify those samples with greater than about 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25% or more minority nucleic acid. In some embodiments, samples determined as having a certain threshold amount of minority nucleic acid (e.g., about 15% or more minority nucleic acid; about 4% or more minority nucleic acid) are further analyzed for a genetic variation/genetic alteration, or the presence or absence of a genetic variation/genetic alteration, for example. In certain embodiments, determinations of, for example, a genetic variation or genetic alteration are selected (e.g., selected and communicated to a patient) only for samples having a certain threshold amount of a minority nucleic acid (e.g., about 15% or more minority nucleic acid; about 4% or more minority nucleic acid).

For example, the amount of cancer cell nucleic acid (e.g., concentration, relative amount, absolute amount, copy number, and the like) in nucleic acid may be determined. In certain instances, the amount of cancer cell nucleic acid in a sample is referred to as “fraction of cancer cell nucleic acid,” and sometimes is referred to as “cancer fraction” or “tumor fraction.” In some embodiments “fraction of cancer cell nucleic acid” refers to the fraction of cancer cell nucleic acid in circulating cell-free nucleic acid in a sample (e.g., a blood sample, a serum sample, a plasma sample, a urine sample) obtained from a subject. Additionally, or alternatively, the amount of fetal nucleic acid (e.g., concentration, relative amount, absolute amount, copy number, and the like) in nucleic acid may be determined. In certain embodiments, the amount of fetal nucleic acid in a sample is referred to as “fetal fraction.” In some embodiments “fetal fraction” refers to the fraction of fetal nucleic acid in circulating cell-free nucleic acid in a sample (e.g., a blood sample, a serum sample, a plasma sample, a urine sample) obtained from a pregnant female. Certain methods described herein or known in the art for determining fetal fraction can be used for determining a fraction of cancer cell nucleic acid and/or a minority species fraction.

In certain instances, fetal fraction may be determined according to markers specific to a male fetus (e.g., Y-chromosome STR markers (e.g., DYS 19, DYS 385, DYS 392 markers); RhD marker in RhD-negative females), allelic ratios of polymorphic sequences, or according to one or more markers specific to fetal nucleic acid and not maternal nucleic acid (e.g., differential epigenetic biomarkers (e.g., methylation) between mother and fetus, or fetal RNA markers in maternal blood plasma (see e.g., Lo, 2005, Journal of Histochemistry and Cytochemistry 53 (3): 293-296)). Determination of fetal fraction sometimes is performed using a fetal quantifier assay (FQA) as described, for example, in U.S. Patent Application Publication No. 2010/0105049, the entire content of which is incorporated herein by reference for all purposes. This type of assay allows for the detection and quantification of fetal nucleic acid in a maternal sample based on the methylation status of the nucleic acid in the sample.

In certain embodiments, a minority species fraction can be determined based on allelic ratios of polymorphic sequences (e.g., single nucleotide polymorphisms (SNPs)), such as, for example, using a method described in U.S. Patent Application Publication No. 2011/0224087, the entire content of which is incorporated herein by reference for all purposes. In such a method for determining fetal fraction, for example, nucleotide sequence reads are obtained for a maternal sample and fetal fraction is determined by comparing the total number of nucleotide sequence reads that map to a first allele and the total number of nucleotide sequence reads that map to a second allele at an informative polymorphic site (e.g., SNP) in a reference genome.

A minority species fraction can be determined, in some embodiments, using methods that incorporate information derived from chromosomal aberrations as described, for example, in International Patent Application Publication No. WO2014/055774, the entire content of which is incorporated herein by reference for all purposes. A minority species fraction can be determined, in some embodiments, using methods that incorporate information derived from sex chromosomes as described, for example, in U.S. Patent Application Publication No. 2013/0288244 and U.S. Patent Application Publication No.

2013/0338933, the entire content of which is incorporated herein by reference for all purposes.

A minority species fraction can be determined in some embodiments using methods that incorporate fragment length information (e.g., fragment length ratio (FLR) analysis, fetal ratio statistic (FRS) analysis as described in International Patent Application Publication No. WO 2013/177086, the entire content of which is incorporated herein by reference for all purposes). Cell-free fetal nucleic acid fragments generally are shorter than maternally-derived nucleic acid fragments (see e.g., Chan et al. (2004) Clin. Chem. 50:88-92; Lo et al. (2010) Sci. Transl. Med. 2:61ra91). Thus, fetal fraction can be determined, in some embodiments, by counting fragments under a particular length threshold and comparing the counts, for example, to counts from fragments over a particular length threshold and/or to the amount of total nucleic acid in the sample. Methods for counting nucleic acid fragments of a particular length are described in further detail in International Patent Application Publication No. WO 2013/177086

A minority species fraction can be determined, in some embodiments, according to portion-specific fraction estimates (e.g., as described in International Patent Application Publication No. WO 2014/205401, the entire content of which is incorporated herein by reference for all purposes). Without being limited to theory, the amount of reads from fetal CCF fragments (e.g., fragments of a particular length, or range of lengths) often map with ranging frequencies to portions (e.g., within the same sample, e.g., within the same sequencing run). Also, without being limited to theory, certain portions, when compared among multiple samples, tend to have a similar representation of reads from fetal CCF fragments (e.g., fragments of a particular length, or range of lengths), and that the representation correlates with portion-specific fetal fractions (e.g., the relative amount, percentage or ratio of CCF fragments originating from a fetus). Portion-specific fetal fraction estimates generally are determined according to portion-specific parameters and their relation to fetal fraction.

In some embodiments, the determination of minority species fraction (e.g., fraction of cancer cell nucleic acid; fetal fraction) is not required or necessary for identifying the presence or absence of a genetic variation or genetic alteration. In some embodiments, identifying the presence or absence of a genetic variation or genetic alteration does not require a sequence differentiation of a minority nucleic acid versus a majority nucleic acid. In certain embodiments, this is because the summed contribution of both minority and majority sequences in a particular chromosome, chromosome portion or part thereof is analyzed. In some embodiments, identifying the presence or absence of a genetic variation or genetic alteration does not rely on a priori sequence information that would distinguish minority nucleic acid from majority nucleic acid.

At block 110, a nucleic acid library is prepared from the isolated nucleic acid or a nucleic acid mixture. In some embodiments a nucleic acid library is a plurality of polynucleotide molecules (e.g., a sample of nucleic acids) that are prepared, assembled and/or modified for a specific process, non-limiting examples of which include immobilization on a solid phase (e.g., a solid support, a flow cell, a bead), enrichment, amplification, cloning, detection and/or for nucleic acid sequencing. In certain embodiments, a nucleic acid library is prepared prior to or during a sequencing process. A nucleic acid library (e.g., sequencing library) can be prepared by a suitable method as known in the art. A nucleic acid library can be prepared by a targeted or a non-targeted preparation process.

In some embodiments a library of nucleic acids is modified to comprise a chemical moiety (e.g., a functional group) configured for immobilization of nucleic acids to a solid support. In certain embodiments a library of nucleic acids is modified to comprise a biomolecule (e.g., a functional group) and/or member of a binding pair configured for immobilization of the library to a solid support, non-limiting examples of which include thyroxin-binding globulin, steroid-binding proteins, antibodies, antigens, haptens, enzymes, lectins, nucleic acids, repressors, protein A, protein G, avidin, streptavidin, biotin, complement component C1q, nucleic acid-binding proteins, receptors, carbohydrates, oligonucleotides, polynucleotides, complementary nucleic acid sequences, the like and combinations thereof. Some examples of specific binding pairs include, without limitation: an avidin moiety and a biotin moiety; an antigenic epitope and an antibody or immunologically reactive fragment thereof; an antibody and a hapten; a digoxigen moiety and an anti-digoxigen antibody; a fluorescein moiety and an anti-fluorescein antibody; an operator and a repressor; a nuclease and a nucleotide; a lectin and a polysaccharide; a steroid and a steroid-binding protein; an active compound and an active compound receptor; a hormone and a hormone receptor; an enzyme and a substrate; an immunoglobulin and protein A; an oligonucleotide or polynucleotide and its corresponding complement; the like or combinations thereof.

In some embodiments, a library of nucleic acids is modified to comprise one or more polynucleotides of known composition, non-limiting examples of which include an identifier (e.g., a tag, an indexing tag), a capture sequence, a label, an adapter, a restriction enzyme site, a promoter, an enhancer, an origin of replication, a stem loop, a complimentary sequence (e.g., a primer binding site, an annealing site), a suitable integration site (e.g., a transposon, a viral integration site), a modified nucleotide, the like or combinations thereof. Polynucleotides of known sequence can be added at a suitable position, for example on the 5′ end, 3′ end or within a nucleic acid sequence. Polynucleotides of known sequence can be the same or different sequences. In some embodiments a polynucleotide of known sequence is configured to hybridize to one or more oligonucleotides immobilized on a surface (e.g., a surface in flow cell). For example, a nucleic acid molecule comprising a 5′ known sequence may hybridize to a first plurality of oligonucleotides while the 3′ known sequence may hybridize to a second plurality of oligonucleotides. In some embodiments a library of nucleic acid can comprise chromosome-specific tags, capture sequences, labels and/or adapters. In some embodiments, a library of nucleic acids comprises one or more detectable labels. In some embodiments one or more detectable labels may be incorporated into a nucleic acid library at a 5′ end, at a 3′ end, and/or at any nucleotide position within a nucleic acid in the library. In some embodiments a library of nucleic acids comprises hybridized oligonucleotides. In certain embodiments hybridized oligonucleotides are labeled probes. In some embodiments a library of nucleic acids comprises hybridized oligonucleotide probes prior to immobilization on a solid phase.

In certain embodiments, a ligation-based library preparation method is used (e.g., ILLUMINA TRUSEQ, Illumina, San Diego Calif.). Ligation-based library preparation methods often make use of an adapter (e.g., a methylated adapter) design which can incorporate an index sequence (e.g., a sample index sequence to identify sample origin for a nucleic acid sequence) at the initial ligation step and often can be used to prepare samples for single-read sequencing, paired-end sequencing and multiplexed sequencing. For example, nucleic acids (e.g., fragmented nucleic acids or cell-free DNA) may be end repaired by a fill-in reaction, an exonuclease reaction or a combination thereof. In some embodiments the resulting blunt-end repaired nucleic acid can then be extended by a single nucleotide, which is complementary to a single nucleotide overhang on the 3′ end of an adapter/primer. Any nucleotide can be used for the extension/overhang nucleotides.

In some embodiments, nucleic acid library preparation comprises ligating an adapter oligonucleotide (e.g., to a sample nucleic acid, to a sample nucleic acid fragment, to a template nucleic acid). Adapter oligonucleotides are often complementary to flow-cell anchors, and sometimes are utilized to immobilize a nucleic acid library to a solid support, such as the inside surface of a flow cell, for example. In some embodiments, an adapter oligonucleotide comprises an identifier, one or more sequencing primer hybridization sites (e.g., sequences complementary to universal sequencing primers, single end sequencing primers, paired end sequencing primers, multiplexed sequencing primers, and the like), or combinations thereof (e.g., adapter/sequencing, adapter/identifier, adapter/identifier/sequencing). In some embodiments, an adapter oligonucleotide comprises one or more of primer annealing polynucleotide (e.g., for annealing to flow cell attached oligonucleotides and/or to free amplification primers), an index polynucleotide (e.g., sample index sequence for tracking nucleic acid from different samples; also referred to as a sample ID), and a barcode polynucleotide (e.g., single molecule barcode (SMB) for tracking individual molecules of sample nucleic acid that are amplified prior to sequencing; also referred to as a molecular barcode). In some embodiments, a primer annealing component of an adapter oligonucleotide comprises one or more universal sequences (e.g., sequences complementary to one or more universal amplification primers). In some embodiments, an index polynucleotide (e.g., sample index; sample ID) is a component of an adapter oligonucleotide. In some embodiments, an index polynucleotide (e.g., sample index; sample ID) is a component of a universal amplification primer sequence.

In some embodiments, adapter oligonucleotides when used in combination with amplification primers (e.g., universal amplification primers) are designed to generate library constructs comprising one or more of: universal sequences, molecular barcodes, sample ID sequences, spacer sequences, and a sample nucleic acid sequence. In some embodiments, adapter oligonucleotides when used in combination with universal amplification primers are designed generate library constructs comprising an ordered combination of one or more of: universal sequences, molecular barcodes, sample ID sequences, spacer sequences, and a sample nucleic acid sequence. For example, a library construct may comprise a first universal sequence, followed by a second universal sequence, followed by first molecular barcode, followed by a spacer sequence, followed by a template sequence (e.g., sample nucleic acid sequence), followed by a spacer sequence, followed by a second molecular barcode, followed by a third universal sequence, followed by a sample ID, followed by a fourth universal sequence. In some embodiments, adapter oligonucleotides when used in combination with amplification primers (e.g., universal amplification primers) are designed generate library constructs for each strand of a template molecule (e.g., sample nucleic acid molecule). In some embodiments, adapter oligonucleotides are duplex adapter oligonucleotides.

A universal sequence is a specific nucleotide sequence that is integrated into two or more nucleic acid molecules or two or more subsets of nucleic acid molecules where the universal sequence is the same for all molecules or subsets of molecules that it is integrated into. A universal sequence is often designed to hybridize to and/or amplify a plurality of different sequences using a single universal primer that is complementary to a universal sequence. In some embodiments two (e.g., a pair) or more universal sequences and/or universal primers are used. A universal primer often comprises a universal sequence. In some embodiments adapters (e.g., universal adapters) comprise universal sequences. In some embodiments one or more universal sequences are used to capture, identify and/or detect multiple species or subsets of nucleic acids.

An identifier can be a suitable detectable label incorporated into or attached to a nucleic acid (e.g., a polynucleotide) that allows detection and/or identification of nucleic acids that comprise the identifier. In some embodiments an identifier is incorporated into or attached to a nucleic acid during a sequencing method (e.g., by a polymerase). Non-limiting examples of identifiers include nucleic acid tags, nucleic acid indexes or barcodes, a radiolabel (e.g., an isotope), metallic label, a fluorescent label, a chemiluminescent label, a phosphorescent label, a fluorophore quencher, a dye, a protein (e.g., an enzyme, an antibody or part thereof, a linker, a member of a binding pair), the like or combinations thereof. In some embodiments an identifier (e.g., a nucleic acid index or barcode) is a unique, known and/or identifiable sequence of nucleotides or nucleotide analogues. In some embodiments identifiers are six or more contiguous nucleotides. A multitude of fluorophores are available with a variety of different excitation and emission spectra. Any suitable type and/or number of fluorophores can be used as an identifier. In some embodiments 1 or more, 2 or more, 3 or more, 4 or more, 5 or more, 6 or more, 7 or more, 8 or more, 9 or more, 10 or more, 20 or more, 30 or more or 50 or more different identifiers are utilized in a method described herein (e.g., a nucleic acid detection and/or sequencing method). In some embodiments, one or two types of identifiers (e.g., fluorescent labels) are linked to each nucleic acid in a library. Detection and/or quantification of an identifier can be performed by a suitable method, apparatus or machine, non-limiting examples of which include flow cytometry, quantitative polymerase chain reaction (qPCR), gel electrophoresis, a luminometer, a fluorometer, a spectrophotometer, a suitable genechip or microarray analysis, Western blot, mass spectrometry, chromatography, cytofluorimetric analysis, fluorescence microscopy, a suitable fluorescence or digital imaging method, confocal laser scanning microscopy, laser scanning cytometry, affinity chromatography, manual batch mode separation, electric field suspension, a suitable nucleic acid sequencing method and/or nucleic acid sequencing apparatus, the like and combinations thereof.

In other embodiments, a transposon-based library preparation method is used (e.g., EPICENTRE NEXTERA, Epicentre, Madison, Wis.). Transposon-based methods typically use in vitro transposition to simultaneously fragment and tag DNA in a single-tube reaction (often allowing incorporation of platform-specific tags and optional barcodes), and prepare sequencer-ready libraries.

In some embodiments, the nucleic acid library or parts thereof are amplified (e.g., amplified by a PCR-based method). For example, a sequencing method may comprise amplification of a nucleic acid library. A nucleic acid library can be amplified prior to or after immobilization on a solid support (e.g., a solid support in a flow cell). Nucleic acid amplification includes the process of amplifying or increasing the numbers of a nucleic acid template and/or of a complement thereof that are present (e.g., in a nucleic acid library), by producing one or more copies of the template and/or its complement. Amplification can be carried out by a suitable method. A nucleic acid library can be amplified by a thermocycling method or by an isothermal amplification method. In some embodiments a rolling circle amplification method is used. In some embodiments amplification takes place on a solid support (e.g., within a flow cell) where a nucleic acid library or portion thereof is immobilized. In certain sequencing methods, a nucleic acid library is added to a flow cell and immobilized by hybridization to anchors under suitable conditions. This type of nucleic acid amplification is often referred to as solid phase amplification. In some embodiments of solid phase amplification, all or a portion of the amplified products are synthesized by an extension initiating from an immobilized primer. Solid phase amplification reactions are analogous to standard solution phase amplifications except that at least one of the amplification oligonucleotides (e.g., primers) is immobilized on a solid support. In some embodiments, modified nucleic acid (e.g., nucleic acid modified by addition of adapters) is amplified.

In some embodiments, solid phase amplification comprises a nucleic acid amplification reaction comprising only one species of oligonucleotide primer immobilized to a surface. In certain embodiments solid phase amplification comprises a plurality of different immobilized oligonucleotide primer species. In some embodiments solid phase amplification may comprise a nucleic acid amplification reaction comprising one species of oligonucleotide primer immobilized on a solid surface and a second different oligonucleotide primer species in solution. Multiple different species of immobilized or solution-based primers can be used. Non-limiting examples of solid phase nucleic acid amplification reactions include interfacial amplification, bridge amplification, emulsion PCR, WildFire amplification (e.g., U.S. Patent Application Publication No. 2013/0012399), the like or combinations thereof.

### 1) Sequencing

At block 115, the nucleic acid (e.g., nucleic acid fragments, sample nucleic acid, cell-free nucleic acid) from the nucleic acid library is sequenced using a sequencing subsystem. In certain instances, a full or substantially full sequence is obtained and sometimes a partial sequence is obtained. Nucleic acid sequencing generally produces a collection of sequence reads. As used herein, “reads” (e.g., “a read,” “a sequence read”) are short nucleotide sequences produced by any sequencing process described herein or known in the art. Reads can be generated from one end of nucleic acid fragments (“single-end reads”), and sometimes are generated from both ends of nucleic acid fragments (e.g., paired-end reads, double-end reads).

The length of a sequence read is often associated with the particular sequencing technology. High-throughput methods, for example, provide sequence reads that can vary in size from tens to hundreds of base pairs (bp). Nanopore sequencing, for example, can provide sequence reads that can vary in size from tens to hundreds to thousands of base pairs. In some embodiments, sequence reads are of a mean, median, average or absolute length of about 15 bp to about 900 bp long. In certain embodiments sequence reads are of a mean, median, average or absolute length of about 1000 bp or more. In some embodiments sequence reads are of a mean, median, average or absolute length of about 1500, 2000, 2500, 3000, 3500, 4000, 4500, or 5000 bp or more. In some embodiments, sequence reads are of a mean, median, average or absolute length of about 100 bp to about 200 bp. In some embodiments, sequence reads are of a mean, median, average or absolute length of about 140 bp to about 160 bp. For example, sequence reads may be of a mean, median, average or absolute length of about 140, 141, 142, 143, 144, 145, 146, 147, 148, 149, 150, 151, 152, 153, 154, 155, 156, 157, 158, 159 or 160 bp.

In some embodiments the nominal, average, mean or absolute length of single-end reads sometimes is about 10 continuous nucleotides to about 250 or more contiguous nucleotides, about 15 contiguous nucleotides to about 200 or more contiguous nucleotides, about 15 contiguous nucleotides to about 150 or more contiguous nucleotides, about 15 contiguous nucleotides to about 125 or more contiguous nucleotides, about 15 contiguous nucleotides to about 100 or more contiguous nucleotides, about 15 contiguous nucleotides to about 75 or more contiguous nucleotides, about 15 contiguous nucleotides to about 60 or more contiguous nucleotides, 15 contiguous nucleotides to about 50 or more contiguous nucleotides, about 15 contiguous nucleotides to about 40 or more contiguous nucleotides, and sometimes about 15 contiguous nucleotides or about 36 or more contiguous nucleotides. In certain embodiments the nominal, average, mean or absolute length of single-end reads is about 20 to about 30 bases, or about 24 to about 28 bases in length. In certain embodiments the nominal, average, mean or absolute length of single-end reads is about 1, 2, 3, 4, 5, 6, 7, 8, 9, 10, 11, 12, 13, 14, 15, 16, 17, 18, 19, 21, 22, 23, 24, 25, 26, 27, 28 or about 29 bases or more in length. In certain embodiments the nominal, average, mean or absolute length of single-end reads is about 20 to about 200 bases, about 100 to about 200 bases, or about 140 to about 160 bases in length. In certain embodiments the nominal, average, mean or absolute length of single-end reads is about 30, 40, 50, 60, 70, 80, 90, 100, 110, 120, 130, 140, 150, 160, 170, 180, 190, or about 200 bases or more in length. In certain embodiments, the nominal, average, mean or absolute length of paired-end reads sometimes is about 10 contiguous nucleotides to about 25 contiguous nucleotides or more (e.g., about 10, 11, 12, 13, 14, 15, 16, 17, 18, 19, 20, 21, 22, 23, 24 or 25 nucleotides in length or more), about 15 contiguous nucleotides to about 20 contiguous nucleotides or more, and sometimes is about 17 contiguous nucleotides or about 18 contiguous nucleotides. In certain embodiments, the nominal, average, mean or absolute length of paired-end reads sometimes is about 25 contiguous nucleotides to about 400 contiguous nucleotides or more (e.g., about 25, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, or 400 nucleotides in length or more), about 50 contiguous nucleotides to about 350 contiguous nucleotides or more, about 100 contiguous nucleotides to about 325 contiguous nucleotides, about 150 contiguous nucleotides to about 325 contiguous nucleotides, about 200 contiguous nucleotides to about 325 contiguous nucleotides, about 275 contiguous nucleotides to about 310 contiguous nucleotides, about 100 contiguous nucleotides to about 200 contiguous nucleotides, about 100 contiguous nucleotides to about 175 contiguous nucleotides, about 125 contiguous nucleotides to about 175 contiguous nucleotides, and sometimes is about 140 contiguous nucleotides to about 160 contiguous nucleotides. In certain embodiments, the nominal, average, mean, or absolute length of paired-end reads is about 150 contiguous nucleotides, and sometimes is 150 contiguous nucleotides.

In some embodiments, nucleotide sequence reads obtained from a sample are partial nucleotide sequence reads. As used herein, “partial nucleotide sequence reads” refers to sequence reads of any length with incomplete sequence information, also referred to as sequence ambiguity. Partial nucleotide sequence reads may lack information regarding nucleobase identity and/or nucleobase position or order. Partial nucleotide sequence reads generally do not include sequence reads in which the only incomplete sequence information (or in which less than all of the bases are sequenced or determined) is from inadvertent or unintentional sequencing errors. Such sequencing errors can be inherent to certain sequencing processes and include, for example, incorrect calls for nucleobase identity, and missing or extra nucleobases. Thus, for partial nucleotide sequence reads herein, certain information about the sequence is often deliberately excluded. That is, one deliberately obtains sequence information with respect to less than all of the nucleobases or which might otherwise be characterized as or be a sequencing error. In some embodiments, a partial nucleotide sequence read can span a portion of a nucleic acid fragment. In some embodiments, a partial nucleotide sequence read can span the entire length of a nucleic acid fragment. Partial nucleotide sequence reads are described, for example, in International Patent Application Publication No. WO 2013/052907, the entire content of which is incorporated herein by reference for all purposes.

Reads generally are representations of nucleotide sequences in a physical nucleic acid. For example, in a read containing an ATGC depiction of a sequence, “A” represents an adenine nucleotide, “T” represents a thymine nucleotide, “G” represents a guanine nucleotide and “C” represents a cytosine nucleotide, in a physical nucleic acid. Sequence reads obtained from a sample from a subject can be reads from a mixture of a minority nucleic acid and a majority nucleic acid. For example, sequence reads obtained from the blood of a cancer patient can be reads from a mixture of cancer nucleic acid and non-cancer nucleic acid. In another example, sequence reads obtained from the blood of a pregnant female can be reads from a mixture of fetal nucleic acid and maternal nucleic acid. A mixture of relatively short reads can be transformed by processes described herein into a representation of genomic nucleic acid present in the subject, and/or a representation of genomic nucleic acid present in a tumor or a fetus. In certain instances, a mixture of relatively short reads can be transformed into a representation of a copy number alteration, a genetic variation/genetic alteration or an aneuploidy, for example. In one example, reads of a mixture of cancer and non-cancer nucleic acid can be transformed into a representation of a composite chromosome or a part thereof comprising features of one or both cancer cell and non-cancer cell chromosomes. In another example, reads of a mixture of maternal and fetal nucleic acid can be transformed into a representation of a composite chromosome or a part thereof comprising features of one or both maternal and fetal chromosomes.

In some instances, circulating cell free nucleic acid fragments (CCF fragments) obtained from a cancer patient comprise nucleic acid fragments originating from normal cells (i.e., non-cancer fragments) and nucleic acid fragments originating from cancer cells (i.e., cancer fragments). Sequence reads derived from CCF fragments originating from normal cells (i.e., non-cancerous cells) are referred to herein as “non-cancer reads.” Sequence reads derived from CCF fragments originating from cancer cells are referred to herein as “cancer reads.” CCF fragments from which non-cancer reads are obtained may be referred to herein as non-cancer templates and CCF fragments from which cancer reads are obtained may be referred herein to as cancer templates.

In some instances, circulating cell free nucleic acid fragments (CCF fragments) obtained from a pregnant female comprise nucleic acid fragments originating from fetal cells (i.e., fetal fragments) and nucleic acid fragments originating from maternal cells (i.e., maternal fragments). Sequence reads derived from CCF fragments originating from a fetus are referred to herein as “fetal reads.” Sequence reads derived from CCF fragments originating from the genome of a pregnant female (e.g., a mother) bearing a fetus are referred to herein as “maternal reads.” CCF fragments from which fetal reads are obtained are referred to herein as fetal templates and CCF fragments from which maternal reads are obtained are referred herein to as maternal templates.

In certain embodiments, “obtaining” nucleic acid sequence reads of a sample from a subject and/or “obtaining” nucleic acid sequence reads of a biological specimen from one or more reference persons can involve directly sequencing nucleic acid to obtain the sequence information. In some embodiments, “obtaining” can involve receiving sequence information obtained directly from a nucleic acid by another.

In some embodiments, some or all nucleic acids in a sample are enriched and/or amplified (e.g., non-specifically, e.g., by a PCR based method) prior to or during sequencing. In certain embodiments specific nucleic acid species or subsets in a sample are enriched and/or amplified prior to or during sequencing. In some embodiments, a species or subset of a pre-selected pool of nucleic acids is sequenced randomly. In some embodiments, nucleic acids in a sample are not enriched and/or amplified prior to or during sequencing.

In some embodiments, a representative fraction of a genome is sequenced and is sometimes referred to as “coverage” or “fold coverage.” For example, a 1-fold coverage indicates that roughly 100% of the nucleotide sequences of the genome are represented by reads. In some instances, fold coverage is referred to as (and is directly proportional to) “sequencing depth.” In some embodiments, “fold coverage” is a relative term referring to a prior sequencing run as a reference. For example, a second sequencing run may have 2-fold less coverage than a first sequencing run. In some embodiments a genome is sequenced with redundancy, where a given region of the genome can be covered by two or more reads or overlapping reads (e.g., a “fold coverage” greater than 1, e.g., a 2-fold coverage). In some embodiments, a genome or a majority of a genome (e.g., genome-wide sequencing) is sequenced with about 0.1-fold to about 100-fold coverage, about 0.2-fold to 20-fold coverage, or about 0.2-fold to about 1-fold coverage (e.g., about 0.2-, 0.3-, 0.4-, 0.5-, 0.6-, 0.7-, 0.8-, 0.9-, 1-, 2-, 3-, 4-, 5-, 6-, 7-, 8-, 9-, 10-, 15-, 20-, 30-, 40-, 50-, 60-, 70-, 80-, 90-fold coverage). In some embodiments, a genome or a majority of a genome (e.g., genome-wide sequencing) is sequenced with about 0.01-fold to about 100-fold coverage, about 0.1-fold to 20-fold coverage, or about 0.1-fold to about 1-fold coverage (e.g., about 0.015-, 0.02-, 0.03-, 0.04-, 0.05-, 0.06-, 0.07-, 0.08-, 0.09-, 0.1-, 0.2-, 0.3-, 0.4-, 0.5-, 0.6-, 0.7-, 0.8-, 0.9-, 1-, 2-, 3-, 4-, 5-, 6-, 7-, 8-, 9-, 10-, 15-, 20-, 30-, 40-, 50-, 60-, 70-, 80-, 90-fold or greater coverage). In some embodiments, specific parts of a genome (e.g., genomic parts from targeted and/or probe-based methods) are sequenced and fold coverage values generally refer to the fraction of the specific genomic parts sequenced (i.e., fold coverage values do not refer to the whole genome). In some instances, specific genomic parts are sequenced at 1000-fold coverage or more. For example, specific genomic parts may be sequenced at 2000-fold, 5,000-fold, 10,000-fold, 20,000-fold, 30,000-fold, 40,000-fold or 50,000-fold coverage. In some embodiments, sequencing is at about 1,000-fold to about 100,000-fold coverage. In some embodiments, sequencing is at about 10,000-fold to about 70,000-fold coverage. In some embodiments, sequencing is at about 20,000-fold to about 60,000-fold coverage. In some embodiments, sequencing is at about 30,000-fold to about 50,000-fold coverage.

In some embodiments, one nucleic acid sample from one individual is sequenced. In certain embodiments, nucleic acids from each of two or more samples are sequenced, where samples are from one individual or from different individuals. In certain embodiments, nucleic acid samples from two or more biological samples are pooled, where each biological sample is from one individual or two or more individuals, and the pool is sequenced. In the latter embodiments, a nucleic acid sample from each biological sample often is identified by one or more unique identifiers.

In some embodiments, a sequencing method utilizes identifiers that allow multiplexing of sequence reactions in a sequencing process. The greater the number of unique identifiers, the greater the number of samples and/or chromosomes for detection, for example, that can be multiplexed in a sequencing process. A sequencing process can be performed using any suitable number of unique identifiers (e.g., 4, 8, 12, 24, 48, 96, 1000, 10000, 108, 1024, or more).

A sequencing process sometimes makes use of a solid phase, and sometimes the solid phase comprises a flow cell on which nucleic acid from a library can be attached and reagents can be flowed and contacted with the attached nucleic acid. A flow cell sometimes includes flow cell lanes, and use of identifiers can facilitate analyzing a number of samples in each lane. A flow cell often is a solid support that can be configured to retain and/or allow the orderly passage of reagent solutions over bound analytes. Flow cells frequently are planar in shape, optically transparent, generally in the millimeter or sub-millimeter scale, and often have channels or lanes in which the analyte/reagent interaction occurs. In some embodiments the number of samples analyzed in a given flow cell lane is dependent on the number of unique identifiers utilized during library preparation and/or probe design. Multiplexing using 12 identifiers, for example, allows simultaneous analysis of 96 samples (e.g., equal to the number of wells in a 96 well microwell plate) in an 8 lane flow cell. Similarly, multiplexing using 48 identifiers, for example, allows simultaneous analysis of 384 samples (e.g., equal to the number of wells in a 384 well microwell plate) in an 8 lane flow cell. Non-limiting examples of commercially available multiplex sequencing kits include Illumina's multiplexing sample preparation oligonucleotide kit and multiplexing sequencing primers and PhiX control kit (e.g., Illumina's catalog numbers PE-400-1001 and PE-400-1002, respectively).

Any suitable method of sequencing nucleic acids can be used, non-limiting examples of which include Maxim & Gilbert, chain-termination methods, sequencing by synthesis, sequencing by ligation, sequencing by mass spectrometry, microscopy-based techniques, the like or combinations thereof. In some embodiments, a first generation technology, such as, for example, Sanger sequencing methods including automated Sanger sequencing methods, including microfluidic Sanger sequencing, can be used in a method provided herein. In some embodiments, sequencing technologies that include the use of nucleic acid imaging technologies (e.g., transmission electron microscopy (TEM) and atomic force microscopy (AFM)), can be used. In some embodiments, a high-throughput sequencing method is used. High-throughput sequencing methods generally involve clonally amplified DNA templates or single DNA molecules that are sequenced in a massively parallel fashion, sometimes within a flow cell. Next generation (e.g., 2nd and 3rd generation) sequencing techniques capable of sequencing DNA in a massively parallel fashion can be used for methods described herein and are collectively referred to herein as “massively parallel sequencing” (MPS). In some embodiments, MPS sequencing methods utilize a targeted approach, where specific chromosomes, genes or regions of interest are sequenced. In certain embodiments, a non-targeted approach is used where most or all nucleic acids in a sample are sequenced, amplified and/or captured randomly.

In some embodiments a targeted enrichment, amplification and/or sequencing approach is used. A targeted approach often isolates, selects and/or enriches a subset of nucleic acids in a sample for further processing by use of sequence-specific oligonucleotides. In some embodiments a library of sequence-specific oligonucleotides are utilized to target (e.g., hybridize to) one or more sets of nucleic acids in a sample. Sequence-specific oligonucleotides and/or primers are often selective for particular sequences (e.g., unique nucleic acid sequences) present in one or more chromosomes, genes, exons, introns, and/or regulatory regions of interest. Any suitable method or combination of methods can be used for enrichment, amplification and/or sequencing of one or more subsets of targeted nucleic acids. In some embodiments targeted sequences are isolated and/or enriched by capture to a solid phase (e.g., a flow cell, a bead) using one or more sequence-specific anchors. In some embodiments targeted sequences are enriched and/or amplified by a polymerase-based method (e.g., a PCR-based method, by any suitable polymerase based extension) using sequence-specific primers and/or primer sets. Sequence specific anchors often can be used as sequence-specific primers.

MPS sequencing sometimes makes use of sequencing by synthesis and certain imaging processes. A nucleic acid sequencing technology that may be used in a method described herein is sequencing-by-synthesis and reversible terminator-based sequencing (e.g., Illumina's Genome Analyzer; Genome Analyzer II; HISEQ 2000; HISEQ 2500 (Illumina, San Diego Calif.)). With this technology, millions of nucleic acid (e.g., DNA) fragments can be sequenced in parallel. In one example of this type of sequencing technology, a flow cell is used which contains an optically transparent slide with 8 individual lanes on the surfaces of which are bound oligonucleotide anchors (e.g., adapter primers).

Sequencing by synthesis generally is performed by iteratively adding (e.g., by covalent addition) a nucleotide to a primer or preexisting nucleic acid strand in a template directed manner. Each iterative addition of a nucleotide is detected and the process is repeated multiple times until a sequence of a nucleic acid strand is obtained. The length of a sequence obtained depends, in part, on the number of addition and detection steps that are performed. In some embodiments of sequencing by synthesis, one, two, three or more nucleotides of the same type (e.g., A, G, C or T) are added and detected in a round of nucleotide addition. Nucleotides can be added by any suitable method (e.g., enzymatically or chemically). For example, in some embodiments a polymerase or a ligase adds a nucleotide to a primer or to a preexisting nucleic acid strand in a template directed manner. In some embodiments of sequencing by synthesis, different types of nucleotides, nucleotide analogues and/or identifiers are used. In some embodiments reversible terminators and/or removable (e.g., cleavable) identifiers are used. In some embodiments fluorescent labeled nucleotides and/or nucleotide analogues are used. In certain embodiments sequencing by synthesis comprises a cleavage (e.g., cleavage and removal of an identifier) and/or a washing step. In some embodiments the addition of one or more nucleotides is detected by a suitable method described herein or known in the art, non-limiting examples of which include any suitable imaging apparatus, a suitable camera, a digital camera, a CCD (Charge Couple Device) based imaging apparatus (e.g., a CCD camera), a CMOS (Complementary Metal Oxide Silicon) based imaging apparatus (e.g., a CMOS camera), a photo diode (e.g., a photomultiplier tube), electron microscopy, a field-effect transistor (e.g., a DNA field-effect transistor), an ISFET ion sensor (e.g., a CHEMFET sensor), the like or combinations thereof.

Any suitable MPS method, system or technology platform for conducting methods described herein can be used to obtain nucleic acid sequence reads. Non-limiting examples of MPS platforms include Illumina/Solex/HiSeq (e.g., Illumina's Genome Analyzer; Genome Analyzer II; HISEQ 2000; HISEQ), SOLID, Roche/454, PACBIO and/or SMRT, Helicos True Single Molecule Sequencing, Ion Torrent and Ion semiconductor-based sequencing (e.g., as developed by Life Technologies), WildFire, 5500, 5500xl W and/or 5500xl W Genetic Analyzer based technologies (e.g., as developed and sold by Life Technologies, U.S. Patent Application Publication No. 2013/0012399); Polony sequencing, Pyrosequencing, Massively Parallel Signature Sequencing (MPSS), RNA polymerase (RNAP) sequencing, LaserGen systems and methods, Nanopore-based platforms, chemical-sensitive field effect transistor (CHEMFET) array, electron microscopy-based sequencing (e.g., as developed by ZS Genetics, Halcyon Molecular), nanoball sequencing, the like or combinations thereof. Other sequencing methods that may be used to conduct methods herein include digital PCR, sequencing by hybridization, nanopore sequencing, chromosome-specific sequencing (e.g., using DANSR (digital analysis of selected regions) technology.

In some embodiments, sequence reads are generated, obtained, gathered, assembled, manipulated, transformed, processed, and/or provided by a sequence subsystem. A machine comprising a sequence subsystem can be a suitable machine and/or apparatus that determines the sequence of a nucleic acid utilizing a sequencing technology known in the art. In some embodiments a sequence subsystem can align, assemble, fragment, complement, reverse complement, and/or error check (e.g., error correct sequence reads).

At block 120, the sequence reads are processed using a sequence processing subsystem to obtain sequence read data. The processing of the sequence reads includes read alignment, mapping, and filtering.

### a) Alignment and Mapping

In some embodiments, one or more processing steps comprises aligning and mapping sequence reads. Sequence reads can be mapped and a number of reads mapping to a specified nucleic acid region (e.g., a chromosome or portion thereof) are referred to as counts. Any suitable mapping method (e.g., process, algorithm, program, software, subsystem, the like or combination thereof) can be used. Certain aspects of mapping processes are described hereafter. Mapping nucleotide sequence reads (i.e., sequence information from a fragment whose physical genomic position is unknown) can be performed in a number of ways, and often comprises alignment of the obtained sequence reads with a matching sequence in a reference genome. In such alignments, sequence reads generally are aligned to a reference sequence and those that align are designated as being “mapped,” as “a mapped sequence read” or as “a mapped read.” In certain embodiments, a mapped sequence read is referred to as a “hit” or “count.” In some embodiments, mapped sequence reads are grouped together according to various parameters and assigned to particular genomic portions, which are discussed in further detail below.

The terms “aligned,” “alignment,” or “aligning” generally refer to two or more nucleic acid sequences that can be identified as a match (e.g., 100% identity) or partial match. Alignments can be done manually or by a computer (e.g., a software, program, subsystem, or algorithm), non-limiting examples of which include the Efficient Local Alignment of Nucleotide Data (ELAND) computer program distributed as part of the Illumina Genomics Analysis pipeline. Alignment of a sequence read can be a 100% sequence match. In some cases, an alignment is less than a 100% sequence match (i.e., non-perfect match, partial match, partial alignment). In some embodiments an alignment is about a 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76% or 75% match. In some embodiments, an alignment comprises a mismatch. In some embodiments, an alignment comprises 1, 2, 3, 4 or 5 mismatches. Two or more sequences can be aligned using either strand (e.g., sense or antisense strand). In certain embodiments a nucleic acid sequence is aligned with the reverse complement of another nucleic acid sequence.

Various computational methods can be used to map each sequence read to a portion. Non-limiting examples of computer algorithms that can be used to align sequences include, without limitation, BLAST, BLITZ, FASTA, BOWTIE 1, BOWTIE 2, ELAND, MAQ, PROBEMATCH, SOAP, BWA or SEQMAP, or variations thereof or combinations thereof. In some embodiments, sequence reads can be aligned with sequences in a reference genome. In some embodiments, sequence reads can be found and/or aligned with sequences in nucleic acid databases known in the art including, for example, GenBank, dbEST, dbSTS, EMBL (European Molecular Biology Laboratory) and DDBJ (DNA Databank of Japan). BLAST or similar tools can be used to search identified sequences against a sequence database. Search hits can then be used to sort the identified sequences into appropriate portions (described hereafter), for example.

In some embodiments, a read may uniquely or non-uniquely map to portions in a reference genome. A read is considered as “uniquely mapped” if it aligns with a single sequence in the reference genome. A read is considered as “non-uniquely mapped” if it aligns with two or more sequences in the reference genome. In some embodiments, non-uniquely mapped reads are eliminated from further analysis (e.g. quantification). A certain, small degree of mismatch (0-1) may be allowed to account for single nucleotide polymorphisms that may exist between the reference genome and the reads from individual samples being mapped, in certain embodiments. In some embodiments, no degree of mismatch is allowed for a read mapped to a reference sequence.

As used herein, the term “reference genome” can refer to any particular known, sequenced or characterized genome, whether partial or complete, of any organism or virus which may be used to reference identified sequences from a subject. For example, a reference genome used for human subjects as well as many other organisms can be found at the National Center for Biotechnology Information at World Wide Web URL ncbi.nlm.nih.gov. A “genome” refers to the complete genetic information of an organism or virus, expressed in nucleic acid sequences. As used herein, a reference sequence or reference genome often is an assembled or partially assembled genomic sequence from an individual or multiple individuals. In some embodiments, a reference genome is an assembled or partially assembled genomic sequence from one or more human individuals. In some embodiments, a reference genome comprises sequences assigned to chromosomes.

In certain embodiments, mappability is assessed for a genomic region (e.g., portion, genomic portion). Mappability is the ability to unambiguously align a nucleotide sequence read to a portion of a reference genome, typically up to a specified number of mismatches, including, for example, 0, 1, 2 or more mismatches. For a given genomic region, the expected mappability can be estimated using a sliding-window approach of a preset read length and averaging the resulting read-level mappability values. Genomic regions comprising stretches of unique nucleotide sequence sometimes have a high mappability value.

For paired-end sequencing, reads may be mapped to a reference genome by use of a suitable mapping and/or alignment program, non-limiting examples of which include BWA (Li H. and Durbin R. (2009) Bioinformatics 25, 1754-60), Novoalign [Novocraft (2010)], Bowtie (Langmead B, et al., (2009) Genome Biol. 10: R25), SOAP2 (Li R, et al., (2009) Bioinformatics 25, 1966-67), BFAST (Homer N, et al., (2009) PLOS ONE 4, e7767), GASSST (Rizk, G. and Lavenier, D. (2010) Bioinformatics 26, 2534-2540), and MPscan (Rivals E., et al. (2009) Lecture Notes in Computer Science 5724, 246-260), and the like. Paired-end reads may be mapped and/or aligned using a suitable short read alignment program. Non-limiting examples of short read alignment programs include BarraCUDA, BFAST, BLASTN, BLAT, Bowtie, BWA, CASHX, CUDA-EC, CUSHAW, CUSHAW2, drFAST, ELAND, ERNE, GNUMAP, GEM, GensearchNGS, GMAP, Geneious Assembler, ISAAC, LAST, MAQ, mrFAST, mrsFAST, MOSAIK, MPscan, Novoalign, NovoalignCS, Novocraft, NextGENe, Omixon, PALMapper, Partek, PASS, PerM, QPalma, RazerS, REAL, CREAL, RMAP, rNA, RTG, Segemehl, SeqMap, Shrec, SHRIMP, SLIDER, SOAP, SOAP2, SOAP3, SOCS, SSAHA, SSAHA2, Stampy, STORM, Subread, Subjunc, Taipan, UGENE, VelociMapper, TimeLogic, XpressAlign, ZOOM, the like or combinations thereof. Paired-end reads are often mapped to opposing ends of the same polynucleotide fragment, according to a reference genome. In some embodiments, read mates are mapped independently. In some embodiments, information from both sequence reads (i.e., from each end) is factored in the mapping process. A reference genome is often used to determine and/or infer the sequence of nucleic acids located between paired-end read mates. The term “discordant read pairs” as used herein refers to a paired-end read comprising a pair of read mates, where one or both read mates fail to unambiguously map to the same region of a reference genome defined, in part, by a segment of contiguous nucleotides. In some embodiments discordant read pairs are paired-end read mates that map to unexpected locations of a reference genome. Non-limiting examples of unexpected locations of a reference genome include (i) two different chromosomes, (ii) locations separated by more than a predetermined fragment size (e.g., more than 300 bp, more than 500 bp, more than 1000 bp, more than 5000 bp, or more than 10,000 bp), (iii) an orientation inconsistent with a reference sequence (e.g., opposite orientations), the like or a combination thereof. In some embodiments discordant read mates are identified according to a length (e.g., an average length, a predetermined fragment size) or expected length of template polynucleotide fragments in a sample. For example, read mates that map to a location that is separated by more than the average length or expected length of polynucleotide fragments in a sample are sometimes identified as discordant read pairs. Read pairs that map in opposite orientation are sometimes determined by taking the reverse complement of one of the reads and comparing the alignment of both reads using the same strand of a reference sequence. Discordant read pairs can be identified by any suitable method and/or algorithm known in the art or described herein (e.g., SVDetect, Lumpy, BreakDancer, BreakDancerMax, CREST, DELLY, the like or combinations thereof).

### b) Portioning

In some embodiments, mapped sequence reads are grouped together according to various parameters and assigned to particular genomic portions (e.g., portions of a reference genome). A “portion” also may be referred to herein as a “genomic section,” “bin,” “partition,” “portion of a reference genome,” “portion of a chromosome” or “genomic portion.” A portion often is defined by partitioning of a genome according to one or more features. Non-limiting examples of certain partitioning features include length (e.g., fixed length, non-fixed length) and other structural features. Genomic portions sometimes include one or more of the following features: fixed length, non-fixed length, random length, non-random length, equal length, unequal length (e.g., at least two of the genomic portions are of unequal length), do not overlap (e.g., the 3′ ends of the genomic portions sometimes abut the 5′ ends of adjacent genomic portions), overlap (e.g., at least two of the genomic portions overlap), contiguous, consecutive, not contiguous, and not consecutive. Genomic portions sometimes are about 1 to about 1,000 kilobases in length (e.g., about 2, 3, 4, 5, 6, 7, 8, 9, 10, 15, 20, 25, 30, 35, 40, 45, 50, 55, 60, 65, 70, 75, 80, 85, 90, 95, 100, 200, 300, 400, 500, 600, 700, 800, 900 kilobases in length), about 5 to about 500 kilobases in length, about 10 to about 100 kilobases in length, or about 40 to about 60 kilobases in length.

Partitioning sometimes is based on, or is based in part on, certain informational features, such as, information content and information gain, for example. Non-limiting examples of certain informational features include speed and/or convenience of alignment, sequencing coverage variability, GC content (e.g., stratified GC content, particular GC contents, high or low GC content), uniformity of GC content, other measures of sequence content (e.g., fraction of individual nucleotides, fraction of pyrimidines or purines, fraction of natural vs. non-natural nucleic acids, fraction of methylated nucleotides, and CpG content), methylation state, duplex melting temperature, amenability to sequencing or PCR, uncertainty value assigned to individual portions of a reference genome, and/or a targeted search for particular features. In some embodiments, information content may be quantified using a p-value profile measuring the significance of particular genomic locations for distinguishing between groups of confirmed normal and abnormal subjects (e.g. euploid and trisomy subjects, respectively).

In some embodiments, partitioning a genome may eliminate similar regions (e.g., identical or homologous regions or sequences) across a genome and only keep unique regions. Regions removed during partitioning may be within a single chromosome, may be one or more chromosomes, or may span multiple chromosomes. In some embodiments, a partitioned genome is reduced and optimized for faster alignment, often focusing on uniquely identifiable sequences.

In some embodiments, genomic portions result from a partitioning based on non-overlapping fixed size, which results in consecutive, non-overlapping portions of fixed length. Such portions often are shorter than a chromosome and often are shorter than a copy number variation (or copy number alteration) region (e.g., a region that is duplicated or is deleted), the latter of which can be referred to as a segment. A “segment” or “genomic segment” often includes two or more fixed-length genomic portions, and often includes two or more consecutive fixed-length portions (e.g., about 2 to about 100 such portions (e.g., 2, 3, 4, 5, 6, 7, 8, 9, 10, 11, 12, 13, 14, 15, 16, 17, 18, 19, 20, 25, 30, 35, 40, 45, 50, 60, 70, 80, 90 such portions)).

Multiple portions sometimes are analyzed in groups, and sometimes reads mapped to portions are quantified according to a particular group of genomic portions. Where portions are partitioned by structural features and correspond to regions in a genome, portions sometimes are grouped into one or more segments and/or one or more regions. Non-limiting examples of regions include sub-chromosome (i.e., shorter than a chromosome), chromosome, autosome, sex chromosome and combinations thereof. One or more sub-chromosome regions sometimes are genes, gene fragments, regulatory sequences, introns, exons, segments (e.g., a segment spanning a copy number alteration region; a segment spanning a copy number variation region), microduplications, microdeletions and the like. A region sometimes is smaller than a chromosome of interest or is the same size of a chromosome of interest, and sometimes is smaller than a reference chromosome or is the same size as a reference chromosome.

c) Filtering and/or Selecting Portions

In some embodiments, one or more processing steps comprise one or more portion filtering steps and/or portion selection steps. The term “filtering” as used herein refers to removing portions or portions of a reference genome from consideration. In certain embodiments one or more portions are filtered (e.g., subjected to a filtering process) thereby providing filtered portions. In some embodiments a filtering process removes certain portions and retains portions (e.g., a subset of portions). Following a filtering process, retained portions are often referred to herein as filtered portions.

Portions of a reference genome can be selected for removal based on any suitable criteria, including but not limited to redundant data (e.g., redundant or overlapping mapped reads), non-informative data (e.g., portions of a reference genome with zero median counts), portions of a reference genome with overrepresented or underrepresented sequences, noisy data, the like, or combinations of the foregoing. A filtering process often involves removing one or more portions of a reference genome from consideration and subtracting the counts in the one or more portions of a reference genome selected for removal from the counted or summed counts for the portions of a reference genome, chromosome or chromosomes, or genome under consideration. In some embodiments, portions of a reference genome can be removed successively (e.g., one at a time to allow evaluation of the effect of removal of each individual portion), and in certain embodiments all portions of a reference genome marked for removal can be removed at the same time. In some embodiments, portions of a reference genome characterized by a variance above or below a certain level are removed, which sometimes is referred to herein as filtering “noisy” portions of a reference genome. In certain embodiments, a filtering process comprises obtaining data points from a data set that deviate from the mean profile level of a portion, a chromosome, or part of a chromosome by a predetermined multiple of the profile variance, and in certain embodiments, a filtering process comprises removing data points from a data set that do not deviate from the mean profile level of a portion, a chromosome or part of a chromosome by a predetermined multiple of the profile variance. In some embodiments, a filtering process is utilized to reduce the number of candidate portions of a reference genome analyzed for the presence or absence of a genetic variation/genetic alteration and/or copy number alteration (e.g., aneuploidy, microdeletion, microduplication). Reducing the number of candidate portions of a reference genome analyzed for the presence or absence of a genetic variation/genetic alteration and/or copy number alteration often reduces the complexity and/or dimensionality of a data set, and sometimes increases the speed of searching for and/or identifying genetic variations/genetic alterations and/or copy number alterations by two or more orders of magnitude.

Portions may be processed (e.g., filtered and/or selected) by any suitable method and according to any suitable parameter. Non-limiting examples of features and/or parameters that can be used to filter and/or select portions include redundant data (e.g., redundant or overlapping mapped reads), non-informative data (e.g., portions of a reference genome with zero mapped counts), portions of a reference genome with over represented or underrepresented sequences, noisy data, counts, count variability, coverage, mappability, variability, a repeatability measure, read density, variability of read density, a level of uncertainty, guanine-cytosine (GC) content, CCF fragment length and/or read length (e.g., a fragment length ratio (FLR), a fetal ratio statistic (FRS)), DNasel-sensitivity, methylation state, acetylation, histone distribution, chromatin structure, percent repeats, the like or combinations thereof. Portions can be filtered and/or selected according to any suitable feature or parameter that correlates with a feature or parameter listed or described herein. Portions can be filtered and/or selected according to features or parameters that are specific to a portion (e.g., as determined for a single portion according to multiple samples) and/or features or parameters that are specific to a sample (e.g., as determined for multiple portions within a sample). In some embodiments portions are filtered and/or removed according to relatively low mappability, relatively high variability, a high level of uncertainty, relatively long CCF fragment lengths (e.g., low FRS, low FLR), relatively large fraction of repetitive sequences, high GC content, low GC content, low counts, zero counts, high counts, the like, or combinations thereof. In some embodiments portions (e.g., a subset of portions) are selected according to suitable level of mappability, variability, level of uncertainty, fraction of repetitive sequences, count, GC content, the like, or combinations thereof. In some embodiments portions (e.g., a subset of portions) are selected according to relatively short CCF fragment lengths (e.g., high FRS, high FLR). Counts and/or reads mapped to portions are sometimes processed (e.g., normalized) prior to and/or after filtering or selecting portions (e.g., a subset of portions). In some embodiments counts and/or reads mapped to portions are not processed prior to and/or after filtering or selecting portions (e.g., a subset of portions).

In some embodiments, portions may be filtered according to a measure of error (e.g., standard deviation, standard error, calculated variance, p-value, mean absolute error (MAE), average absolute deviation and/or mean absolute deviation (MAD)). In certain instances, a measure of error may refer to count variability. In some embodiments portions are filtered according to count variability. In certain embodiments count variability is a measure of error determined for counts mapped to a portion (i.e., portion) of a reference genome for multiple samples (e.g., multiple sample obtained from multiple subjects, e.g., 50 or more, 100 or more, 500 or more 1000 or more, 5000 or more or 10,000 or more subjects). In some embodiments, portions with a count variability above a pre-determined upper range are filtered (e.g., excluded from consideration). In some embodiments portions with a count variability below a pre-determined lower range are filtered (e.g., excluded from consideration). In some embodiments, portions with a count variability outside a pre-determined range are filtered (e.g., excluded from consideration). In some embodiments portions with a count variability within a pre-determined range are selected (e.g., used for determining the presence or absence of a copy number alteration). In some embodiments, count variability of portions represents a distribution (e.g., a normal distribution). In some embodiments portions are selected within a quantile of the distribution. In some embodiments portions within a 99% quantile of the distribution of count variability are selected.

Sequence reads from any suitable number of samples can be utilized to identify a subset of portions that meet one or more criteria, parameters and/or features described herein. Sequence reads from a group of samples from multiple subjects sometimes are utilized. In some embodiments, the multiple subjects include pregnant females. In some embodiments, the multiple subjects include healthy subjects. In some embodiments, the multiple subjects include cancer patients. One or more samples from each of the multiple subjects can be addressed (e.g., 1 to about 20 samples from each subject (e.g., about 2, 3, 4, 5, 6, 7, 8, 9, 10, 11, 12, 13, 14, 15, 16, 17, 18 or 19 samples)), and a suitable number of subjects may be addressed (e.g., about 2 to about 10,000 subjects (e.g., about 10, 20, 30, 40, 50, 60, 70, 80, 90, 100, 150, 200, 250, 300, 350, 400, 500, 600, 700, 800, 900, 1000, 2000, 3000, 4000, 5000, 6000, 7000, 8000, 9000 subjects)). In some embodiments, sequence reads from the same test sample(s) from the same subject are mapped to portions in the reference genome and are used to generate the subset of portions.

Portions can be selected and/or filtered by any suitable method. In some embodiments portions are selected according to visual inspection of data, graphs, plots and/or charts. In certain embodiments portions are selected and/or filtered (e.g., in part) by a system or a machine comprising one or more microprocessors and memory. In some embodiments portions are selected and/or filtered (e.g., in part) by a non-transitory computer-readable storage medium with an executable program stored thereon, where the program instructs a microprocessor to perform the selecting and/or filtering.

In some embodiments, sequence reads derived from a sample are mapped to all or most portions of a reference genome and a pre-selected subset of portions are thereafter selected. For example, a subset of portions to which reads from fragments under a particular length threshold preferentially map may be selected. Certain methods for pre-selecting a subset of portions are described in U.S. Patent Application Publication No. 2014/0180594, the entire content of which is incorporated herein by reference for all purposes. Reads from a selected subset of portions often are utilized in further steps of a determination of the presence or absence of a genetic variation or genetic alteration, for example. Often, reads from portions not selected are not utilized in further steps of a determination of the presence or absence of a genetic variation or genetic alteration (e.g., reads in the non-selected portions are removed or filtered).

In some embodiments, portions associated with read densities (e.g., where a read density is for a portion) are removed by a filtering process and read densities associated with removed portions are not included in a determination of the presence or absence of a copy number alteration (e.g., a chromosome aneuploidy, microduplication, microdeletion). In some embodiments a read density profile comprises and/or consists of read densities of filtered portions. Portions are sometimes filtered according to a distribution of counts and/or a distribution of read densities. In some embodiments portions are filtered according to a distribution of counts and/or read densities where the counts and/or read densities are obtained from one or more reference samples. One or more reference samples may be referred to herein as a training set. In some embodiments portions are filtered according to a distribution of counts and/or read densities where the counts and/or read densities are obtained from one or more test samples. In some embodiments portions are filtered according to a measure of uncertainty for a read density distribution. In certain embodiments, portions that demonstrate a large deviation in read densities are removed by a filtering process. For example, a distribution of read densities (e.g., a distribution of average mean, or median read densities) can be determined, where each read density in the distribution maps to the same portion. A measure of uncertainty (e.g., a MAD) can be determined by comparing a distribution of read densities for multiple samples where each portion of a genome is associated with measure of uncertainty. According to the foregoing example, portions can be filtered according to a measure of uncertainty (e.g., a standard deviation (SD), a MAD) associated with each portion and a predetermined threshold. In certain instances, portions comprising MAD values within the acceptable range are retained and portions comprising MAD values outside of the acceptable range are removed from consideration by a filtering process. In some embodiments, according to the foregoing example, portions comprising read densities values (e.g., median, average or mean read densities) outside a pre-determined measure of uncertainty are often removed from consideration by a filtering process. In some embodiments portions comprising read densities values (e.g., median, average or mean read densities) outside an inter-quartile range of a distribution are removed from consideration by a filtering process. In some embodiments portions comprising read densities values outside more than 2 times, 3 times, 4 times or 5 times an inter-quartile range of a distribution are removed from consideration by a filtering process. In some embodiments portions comprising read densities values outside more than 2 sigma, 3 sigma, 4 sigma, 5 sigma, 6 sigma, 7 sigma or 8 sigma (e.g., where sigma is a range defined by a standard deviation) are removed from consideration by a filtering process.

### 2) Data Processing

At block 125, the sequence read data is processed using a data processing subsystem to obtain processed sequence read data. The processing of the sequence read data includes quantification, assignment of levels or classes, data normalization, or any combination thereof.

### a) Sequence Read Quantification

Sequence reads that are mapped or partitioned based on a selected feature or variable can be quantified to determine the amount or number of reads that are mapped to one or more portions (e.g., portion of a reference genome), in some embodiments. In certain embodiments the quantity of sequence reads that are mapped to a portion or segment is referred to as a count or read density. A count often is associated with a genomic portion. In some embodiments a count is determined from some or all of the sequence reads mapped to (i.e., associated with) a portion. In certain embodiments, a count is determined from some or all of the sequence reads mapped to a group of portions (e.g., portions in a segment or region (described herein)).

A count can be determined by a suitable method, operation or mathematical process. A count sometimes is the direct sum of all sequence reads mapped to a genomic portion or a group of genomic portions corresponding to a segment, a group of portions corresponding to a sub-region of a genome (e.g., copy number variation region, copy number alteration region, copy number duplication region, copy number deletion region, microduplication region, microdeletion region, chromosome region, autosome region, sex chromosome region or other chromosomal rearrangement) and/or sometimes is a group of portions corresponding to a genome. A read quantification sometimes is a ratio, and sometimes is a ratio of a quantification for portion(s) in region a to a quantification for portion(s) in region b. Region a sometimes is one portion, segment region, copy number variation region, copy number alteration region, copy number duplication region, copy number deletion region, microduplication region, microdeletion region, chromosome region, autosome region and/or sex chromosome region. Region b independently sometimes is one portion, segment region, copy number variation region, copy number alteration region, copy number duplication region, copy number deletion region, microduplication region, microdeletion region, chromosome region, autosome region, sex chromosome region, a region including all autosomes, a region including sex chromosomes and/or a region including all chromosomes.

In some embodiments, a count is derived from raw sequence reads and/or filtered sequence reads. In certain embodiments a count is determined by a mathematical process. In certain embodiments a count is an average, mean or sum of sequence reads mapped to a genomic portion or group of genomic portions (e.g., genomic portions in a region). In some embodiments, a count is associated with an uncertainty value. A count sometimes is adjusted. A count may be adjusted according to sequence reads associated with a genomic portion or group of portions that have been weighted, removed, filtered, normalized, adjusted, averaged, derived as a mean, derived as a median, added, or combination thereof.

A sequence read quantification sometimes is a read density. A read density may be determined and/or generated for one or more segments of a genome. In certain instances, a read density may be determined and/or generated for one or more chromosomes. In some embodiments a read density comprises a quantitative measure of counts of sequence reads mapped to a segment or portion of a reference genome. A read density can be determined by a suitable process. In some embodiments a read density is determined by a suitable distribution and/or a suitable distribution function. Non-limiting examples of a distribution function include a probability function, probability distribution function, probability density function (PDF), a kernel density function (kernel density estimation), a cumulative distribution function, probability mass function, discrete probability distribution, an absolutely continuous univariate distribution, the like, any suitable distribution, or combinations thereof. A read density may be a density estimation derived from a suitable probability density function. A density estimation is the construction of an estimate, based on observed data, of an underlying probability density function. In some embodiments a read density comprises a density estimation (e.g., a probability density estimation, a kernel density estimation). A read density may be generated according to a process comprising generating a density estimation for each of the one or more portions of a genome where each portion comprises counts of sequence reads. A read density may be generated for normalized and/or weighted counts mapped to a portion or segment. In some instances, each read mapped to a portion or segment may contribute to a read density, a value (e.g., a count) equal to its weight obtained from a normalization process described herein. In some embodiments read densities for one or more portions or segments are adjusted. Read densities can be adjusted by a suitable method. For example, read densities for one or more portions can be weighted and/or normalized.

Reads quantified for a given portion or segment can be from one source or different sources. In one example, reads may be obtained from nucleic acid from a subject having cancer or suspected of having cancer. In such circumstances, reads mapped to one or more portions often are reads representative of both healthy cells (i.e., non-cancer cells) and cancer cells (e.g., tumor cells). In certain embodiments, some of the reads mapped to a portion are from cancer cell nucleic acid and some of the reads mapped to the same portion are from non-cancer cell nucleic acid. In another example, reads may be obtained from a nucleic acid sample from a pregnant female bearing a fetus. In such circumstances, reads mapped to one or more portions often are reads representative of both the fetus and the mother of the fetus (e.g., a pregnant female subject). In certain embodiments some of the reads mapped to a portion are from a fetal genome and some of the reads mapped to the same portion are from a maternal genome.

### b) Levels

In some embodiments, a value (e.g., a number, a quantitative value) is ascribed to a level. A level can be determined by a suitable method, operation or mathematical process (e.g., a processed level). A level often is, or is derived from, counts (e.g., normalized counts) for a set of portions. In some embodiments a level of a portion is substantially equal to the total number of counts mapped to a portion (e.g., counts, normalized counts). Often a level is determined from counts that are processed, transformed or manipulated by a suitable method, operation or mathematical process known in the art. In some embodiments a level is derived from counts that are processed and non-limiting examples of processed counts include weighted, removed, filtered, normalized, adjusted, averaged, derived as a mean (e.g., mean level), added, subtracted, transformed counts or combination thereof. In some embodiments a level comprises counts that are normalized (e.g., normalized counts of portions). A level can be for counts normalized by a suitable process, non-limiting examples of which are described herein. A level can comprise normalized counts or relative amounts of counts. In some embodiments a level is for counts or normalized counts of two or more portions that are averaged and the level is referred to as an average level. In some embodiments a level is for a set of portions having a mean count or mean of normalized counts which is referred to as a mean level. In some embodiments a level is derived for portions that comprise raw and/or filtered counts. In some embodiments, a level is based on counts that are raw. In some embodiments a level is associated with an uncertainty value (e.g., a standard deviation, a MAD). In some embodiments a level is represented by a Z-score or p-value.

A level for one or more portions is synonymous with a “genomic section level” herein. The term “level” as used herein is sometimes synonymous with the term “elevation.” A determination of the meaning of the term “level” can be determined from the context in which it is used. For example, the term “level,” when used in the context of portions, profiles, reads and/or counts often means an elevation. The term “level,” when used in the context of a substance or composition (e.g., level of RNA, plexing level) often refers to an amount. The term “level,” when used in the context of uncertainty (e.g., level of error, level of confidence, level of deviation, level of uncertainty) often refers to an amount.

Normalized or non-normalized counts for two or more levels (e.g., two or more levels in a profile) can sometimes be mathematically manipulated (e.g., added, multiplied, averaged, normalized, the like or combination thereof) according to levels. For example, normalized or non-normalized counts for two or more levels can be normalized according to one, some or all of the levels in a profile. In some embodiments normalized or non-normalized counts of all levels in a profile are normalized according to one level in the profile. In some embodiments normalized or non-normalized counts of a first level in a profile are normalized according to normalized or non-normalized counts of a second level in the profile.

Non-limiting examples of a level (e.g., a first level, a second level) are a level for a set of portions comprising processed counts, a level for a set of portions comprising a mean, median or average of counts, a level for a set of portions comprising normalized counts, the like or any combination thereof. In some embodiments, a first level and a second level in a profile are derived from counts of portions mapped to the same chromosome. In some embodiments, a first level and a second level in a profile are derived from counts of portions mapped to different chromosomes.

In some embodiments a level is determined from normalized or non-normalized counts mapped to one or more portions. In some embodiments, a level is determined from normalized or non-normalized counts mapped to two or more portions, where the normalized counts for each portion often are about the same. There can be variation in counts (e.g., normalized counts) in a set of portions for a level. In a set of portions for a level there can be one or more portions having counts that are significantly different than in other portions of the set (e.g., peaks and/or dips). Any suitable number of normalized or non-normalized counts associated with any suitable number of portions can define a level.

In some embodiments one or more levels can be determined from normalized or non-normalized counts of all or some of the portions of a genome. Often a level can be determined from all or some of the normalized or non-normalized counts of a chromosome, or part thereof. In some embodiments, two or more counts derived from two or more portions (e.g., a set of portions) determine a level. In some embodiments two or more counts (e.g., counts from two or more portions) determine a level. In some embodiments, counts from 2 to about 100,000 portions determine a level. In some embodiments, counts from 2 to about 50,000, 2 to about 40,000, 2 to about 30,000, 2 to about 20,000, 2 to about 10,000, 2 to about 5000, 2 to about 2500, 2 to about 1250, 2 to about 1000, 2 to about 500, 2 to about 250, 2 to about 100 or 2 to about 60 portions determine a level. In some embodiments counts from about 10 to about 50 portions determine a level. In some embodiments counts from about 20 to about 40 or more portions determine a level. In some embodiments, a level comprises counts from about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60 or more portions. In some embodiments, a level corresponds to a set of portions (e.g., a set of portions of a reference genome, a set of portions of a chromosome or a set of portions of a part of a chromosome).

In some embodiments, a level is determined for normalized or non-normalized counts of portions that are contiguous. In some embodiments portions (e.g., a set of portions) that are contiguous represent neighboring regions of a genome or neighboring regions of a chromosome or gene. For example, two or more contiguous portions, when aligned by merging the portions end to end, can represent a sequence assembly of a DNA sequence longer than each portion. For example, two or more contiguous portions can represent of an intact genome, chromosome, gene, intron, exon or part thereof. In some embodiments a level is determined from a collection (e.g., a set) of contiguous portions and/or non-contiguous portions.

### c) Data Processing and Normalization

Mapped sequence reads that have been counted are referred to herein as raw data, since the data represents unmanipulated counts (e.g., raw counts). In some embodiments, sequence read data in a data set can be processed further (e.g., mathematically and/or statistically manipulated) and/or displayed to facilitate providing an outcome. In certain embodiments, data sets, including larger data sets, may benefit from pre-processing to facilitate further analysis. Pre-processing of data sets sometimes involves removal of redundant and/or uninformative portions or portions of a reference genome (e.g., portions of a reference genome with uninformative data, redundant mapped reads, portions with zero median counts, overrepresented or underrepresented sequences). Without being limited by theory, data processing and/or preprocessing may (i) remove noisy data, (ii) remove uninformative data, (iii) remove redundant data, (iv) reduce the complexity of larger data sets, and/or (v) facilitate transformation of the data from one form into one or more other forms. The terms “pre-processing” and “processing” when utilized with respect to data or data sets are collectively referred to herein as “processing.” Processing can render data more amenable to further analysis, and can generate an outcome in some embodiments. In some embodiments one or more or all processing methods (e.g., normalization methods, portion filtering, mapping, validation, the like or combinations thereof) are performed by a processor, a microprocessor, a computer, in conjunction with memory and/or by a microprocessor controlled apparatus.

The term “noisy data” as used herein refers to (a) data that has a significant variance between data points when analyzed or plotted, (b) data that has a significant standard deviation (e.g., greater than 3 standard deviations), (c) data that has a significant standard error of the mean, the like, and combinations of the foregoing. Noisy data sometimes occurs due to the quantity and/or quality of starting material (e.g., nucleic acid sample), and sometimes occurs as part of processes for preparing or replicating DNA used to generate sequence reads. In certain embodiments, noise results from certain sequences being overrepresented when prepared using PCR-based methods. Methods described herein can reduce or eliminate the contribution of noisy data, and therefore reduce the effect of noisy data on the provided outcome.

The terms “uninformative data,” “uninformative portions of a reference genome,” and “uninformative portions” as used herein refer to portions, or data derived therefrom, having a numerical value that is significantly different from a predetermined threshold value or falls outside a predetermined cutoff range of values. The terms “threshold” and “threshold value” herein refer to any number that is calculated using a qualifying data set and serves as a limit of diagnosis of a genetic variation or genetic alteration (e.g., a copy number alteration, an aneuploidy, a microduplication, a microdeletion, a chromosomal aberration, and the like). In certain embodiments, a threshold is exceeded by results obtained by methods described herein and a subject is diagnosed with a copy number alteration. A threshold value or range of values often is calculated by mathematically and/or statistically manipulating sequence read data (e.g., from a reference and/or subject), in some embodiments, and in certain embodiments, sequence read data manipulated to generate a threshold value or range of values is sequence read data (e.g., from a reference and/or subject). In some embodiments, an uncertainty value is determined. An uncertainty value generally is a measure of variance or error and can be any suitable measure of variance or error. In some embodiments an uncertainty value is a standard deviation, standard error, calculated variance, p-value, or mean absolute deviation (MAD). In some embodiments an uncertainty value can be calculated according to a formula described herein.

Any suitable procedure can be utilized for processing data sets described herein. Non-limiting examples of procedures suitable for use for processing data sets include filtering, normalizing, weighting, monitoring peak heights, monitoring peak areas, monitoring peak edges, peak level analysis, peak width analysis, peak edge location analysis, peak lateral tolerances, determining area ratios, mathematical processing of data, statistical processing of data, application of statistical algorithms, analysis with fixed variables, analysis with optimized variables, plotting data to identify patterns or trends for additional processing, the like and combinations of the foregoing. In some embodiments, data sets are processed based on various features (e.g., GC content, redundant mapped reads, centromere regions, telomere regions, the like and combinations thereof) and/or variables (e.g., subject sex, subject age, subject ploidy, percent contribution of cancer cell nucleic acid, fetal sex, maternal age, maternal ploidy, percent contribution of fetal nucleic acid, the like or combinations thereof). In certain embodiments, processing data sets as described herein can reduce the complexity and/or dimensionality of large and/or complex data sets. A non-limiting example of a complex data set includes sequence read data generated from one or more test subjects and a plurality of reference subjects of different ages and ethnic backgrounds. In some embodiments, data sets can include from thousands to millions of sequence reads for each test and/or reference subject.

Data processing can be performed in any number of steps, in certain embodiments. For example, data may be processed using only a single processing procedure in some embodiments, and in certain embodiments data may be processed using 1 or more, 5 or more, 10 or more or 20 or more processing steps (e.g., 1 or more processing steps, 2 or more processing steps, 3 or more processing steps, 4 or more processing steps, 5 or more processing steps, 6 or more processing steps, 7 or more processing steps, 8 or more processing steps, 9 or more processing steps, 10 or more processing steps, 11 or more processing steps, 12 or more processing steps, 13 or more processing steps, 14 or more processing steps, 15 or more processing steps, 16 or more processing steps, 17 or more processing steps, 18 or more processing steps, 19 or more processing steps, or 20 or more processing steps). In some embodiments, processing steps may be the same step repeated two or more times (e.g., filtering two or more times, normalizing two or more times), and in certain embodiments, processing steps may be two or more different processing steps (e.g., filtering, normalizing; normalizing, monitoring peak heights and edges; filtering, normalizing, normalizing to a reference, statistical manipulation to determine p-values, and the like), carried out simultaneously or sequentially. In some embodiments, any suitable number and/or combination of the same or different processing steps can be utilized to process sequence read data to facilitate providing an outcome. In certain embodiments, processing data sets by the criteria described herein may reduce the complexity and/or dimensionality of a data set.

In some embodiments one or more processing steps can comprise one or more normalization steps. Normalization can be performed by a suitable method described herein or known in the art. In certain embodiments, normalization comprises adjusting values measured on different scales to a notionally common scale. In certain embodiments, normalization comprises a sophisticated mathematical adjustment to bring probability distributions of adjusted values into alignment. In some embodiments normalization comprises aligning distributions to a normal distribution. In certain embodiments normalization comprises mathematical adjustments that allow comparison of corresponding normalized values for different datasets in a way that eliminates the effects of certain gross influences (e.g., error and anomalies). In certain embodiments normalization comprises scaling. Normalization sometimes comprises division of one or more data sets by a predetermined variable or formula. Normalization sometimes comprises subtraction of one or more data sets by a predetermined variable or formula. Non-limiting examples of normalization methods include portion-wise normalization, normalization by GC content, median count (median bin count, median portion count) normalization, linear and nonlinear least squares regression, LOESS, GC LOESS, LOWESS (locally weighted scatterplot smoothing), principal component normalization, repeat masking (RM), GC-normalization and repeat masking (GCRM), cQn and/or combinations thereof. In some embodiments, the determination of a presence or absence of a copy number alteration (e.g., an aneuploidy, a microduplication, a microdeletion) utilizes a normalization method (e.g., portion-wise normalization, normalization by GC content, median count (median bin count, median portion count) normalization, linear and nonlinear least squares regression, LOESS, GC LOESS, LOWESS (locally weighted scatterplot smoothing), principal component normalization, repeat masking (RM), GC-normalization and repeat masking (GCRM), cQn, a normalization method known in the art and/or a combination thereof). Described in greater detail hereafter are certain examples of normalization processes that can be utilized, such as LOESS normalization, principal component normalization, and hybrid normalization methods, for example. Aspects of certain normalization processes also are described, for example, in International Patent Application Publication No. WO 2013/052913 and International Patent Application Publication No. WO 2015/051163, the entire content of which is incorporated herein by reference for all purposes.

Any suitable number of normalizations can be used. In some embodiments, data sets can be normalized 1 or more, 5 or more, 10 or more or even 20 or more times. Data sets can be normalized to values (e.g., normalizing value) representative of any suitable feature or variable (e.g., sample data, reference data, or both). Non-limiting examples of types of data normalizations that can be used include normalizing raw count data for one or more selected test or reference portions to the total number of counts mapped to the chromosome or the entire genome on which the selected portion or sections are mapped; normalizing raw count data for one or more selected portions to a median reference count for one or more portions or the chromosome on which a selected portion is mapped; normalizing raw count data to previously normalized data or derivatives thereof; and normalizing previously normalized data to one or more other predetermined normalization variables. Normalizing a data set sometimes has the effect of isolating statistical error, depending on the feature or property selected as the predetermined normalization variable. Normalizing a data set sometimes also allows comparison of data characteristics of data having different scales, by bringing the data to a common scale (e.g., predetermined normalization variable). In some embodiments, one or more normalizations to a statistically derived value can be utilized to minimize data differences and diminish the importance of outlying data. Normalizing portions, or portions of a reference genome, with respect to a normalizing value sometimes is referred to as “portion-wise normalization.”

In certain embodiments, a processing step can comprise one or more mathematical and/or statistical manipulations. Any suitable mathematical and/or statistical manipulation, alone or in combination, may be used to analyze and/or manipulate a data set described herein. Any suitable number of mathematical and/or statistical manipulations can be used. In some embodiments, a data set can be mathematically and/or statistically manipulated 1 or more, 5 or more, 10 or more or 20 or more times. Non-limiting examples of mathematical and statistical manipulations that can be used include addition, subtraction, multiplication, division, algebraic functions, least squares estimators, curve fitting, differential equations, rational polynomials, double polynomials, orthogonal polynomials, z-scores, p-values, chi values, phi values, analysis of peak levels, determination of peak edge locations, calculation of peak area ratios, analysis of median chromosomal level, calculation of mean absolute deviation, sum of squared residuals, mean, standard deviation, standard error, the like or combinations thereof. A mathematical and/or statistical manipulation can be performed on all or a portion of sequence read data, or processed products thereof. Non-limiting examples of data set variables or features that can be statistically manipulated include raw counts, filtered counts, normalized counts, peak heights, peak widths, peak areas, peak edges, lateral tolerances, P-values, median levels, mean levels, count distribution within a genomic region, relative representation of nucleic acid species, the like or combinations thereof.

In some embodiments, a processing step can comprise the use of one or more statistical algorithms. Any suitable statistical algorithm, alone or in combination, may be used to analyze and/or manipulate a data set described herein. Any suitable number of statistical algorithms can be used. In some embodiments, a data set can be analyzed using 1 or more, 5 or more, 10 or more or 20 or more statistical algorithms. Non-limiting examples of statistical algorithms suitable for use with methods described herein include principal component analysis, decision trees, counternulls, multiple comparisons, omnibus test, Behrens-Fisher problem, bootstrapping, Fisher's method for combining independent tests of significance, null hypothesis, type I error, type II error, exact test, one-sample Z test, two-sample Z test, one-sample t-test, paired t-test, two-sample pooled t-test having equal variances, two-sample unpooled t-test having unequal variances, one-proportion z-test, two-proportion z-test pooled, two-proportion z-test unpooled, one-sample chi-square test, two-sample F test for equality of variances, confidence interval, credible interval, significance, meta analysis, simple linear regression, robust linear regression, the like or combinations of the foregoing. Non-limiting examples of data set variables or features that can be analyzed using statistical algorithms include raw counts, filtered counts, normalized counts, peak heights, peak widths, peak edges, lateral tolerances, P-values, median levels, mean levels, count distribution within a genomic region, relative representation of nucleic acid species, the like or combinations thereof.

In certain embodiments, a data set can be analyzed by utilizing multiple (e.g., 2 or more) statistical algorithms (e.g., least squares regression, principal component analysis, linear discriminant analysis, quadratic discriminant analysis, bagging, neural networks, support vector machine models, random forests, classification tree models, K-nearest neighbors, logistic regression and/or smoothing) and/or mathematical and/or statistical manipulations (e.g., referred to herein as manipulations). The use of multiple manipulations can generate an N-dimensional space that can be used to provide an outcome, in some embodiments. In certain embodiments, analysis of a data set by utilizing multiple manipulations can reduce the complexity and/or dimensionality of the data set. For example, the use of multiple manipulations on a reference data set can generate an N-dimensional space (e.g., probability plot) that can be used to represent the presence or absence of a genetic variation/genetic alteration and/or copy number alteration, depending on the status of the reference samples (e.g., positive or negative for a selected copy number alteration). Analysis of test samples using a substantially similar set of manipulations can be used to generate an N-dimensional point for each of the test samples. The complexity and/or dimensionality of a test subject data set sometimes is reduced to a single value or N-dimensional point that can be readily compared to the N-dimensional space generated from the reference data. Test sample data that fall within the N-dimensional space populated by the reference subject data are indicative of a genetic status substantially similar to that of the reference subjects. Test sample data that fall outside of the N-dimensional space populated by the reference subject data are indicative of a genetic status substantially dissimilar to that of the reference subjects. In some embodiments, references are euploid or do not otherwise have a genetic variation/genetic alteration and/or copy number alteration and/or medical condition.

After data sets have been counted, optionally filtered, normalized, and optionally weighted the processed data sets can be further manipulated by one or more filtering and/or normalizing and/or weighting procedures, in some embodiments. A data set that has been further manipulated by one or more filtering and/or normalizing and/or weighting procedures can be used to generate a profile, in certain embodiments. The one or more filtering and/or normalizing and/or weighting procedures sometimes can reduce data set complexity and/or dimensionality, in some embodiments. An outcome can be provided based on a data set of reduced complexity and/or dimensionality. In some embodiments, a profile plot of processed data further manipulated by weighting, for example, is generated to facilitate classification and/or providing an outcome. An outcome can be provided based on a profile plot of weighted data, for example.

Filtering or weighting of portions can be performed at one or more suitable points in an analysis. For example, portions may be filtered or weighted before or after sequence reads are mapped to portions of a reference genome. Portions may be filtered or weighted before or after an experimental bias for individual genome portions is determined in some embodiments. In certain embodiments, portions may be filtered or weighted before or after levels are calculated.

After data sets have been counted, optionally filtered, normalized, and optionally weighted, the processed data sets can be manipulated by one or more mathematical and/or statistical (e.g., statistical functions or statistical algorithm) manipulations, in some embodiments. In certain embodiments, processed data sets can be further manipulated by calculating Z-scores for one or more selected portions, chromosomes, or portions of chromosomes. In some embodiments, processed data sets can be further manipulated by calculating P-values. In certain embodiments, mathematical and/or statistical manipulations include one or more assumptions pertaining to ploidy and/or fraction of a minority species (e.g., fraction of cancer cell nucleic acid; fetal fraction). In some embodiments, a profile plot of processed data further manipulated by one or more statistical and/or mathematical manipulations is generated to facilitate classification and/or providing an outcome. An outcome can be provided based on a profile plot of statistically and/or mathematically manipulated data. An outcome provided based on a profile plot of statistically and/or mathematically manipulated data often includes one or more assumptions pertaining to ploidy and/or fraction of a minority species (e.g., fraction of cancer cell nucleic acid; fetal fraction).

In some embodiments, analysis and processing of data can include the use of one or more assumptions. A suitable number or type of assumptions can be utilized to analyze or process a data set. Non-limiting examples of assumptions that can be used for data processing and/or analysis include subject ploidy, cancer cell contribution, maternal ploidy, fetal contribution, prevalence of certain sequences in a reference population, ethnic background, prevalence of a selected medical condition in related family members, parallelism between raw count profiles from different patients and/or runs after GC-normalization and repeat masking (e.g., GCRM), identical matches represent PCR artifacts (e.g., identical base position), assumptions inherent in a nucleic acid quantification assay (e.g., fetal quantifier assay (FQA)), assumptions regarding twins (e.g., if 2 twins and only 1 is affected the effective fetal fraction is only 50% of the total measured fetal fraction (similarly for triplets, quadruplets and the like)), cell free DNA (e.g., cfDNA) uniformly covers the entire genome, the like and combinations thereof.

In those instances where the quality and/or depth of mapped sequence reads does not permit an outcome prediction of the presence or absence of a genetic variation/genetic alteration and/or copy number alteration at a desired confidence level (e.g., 95% or higher confidence level), based on the normalized count profiles, one or more additional mathematical manipulation algorithms and/or statistical prediction algorithms, can be utilized to generate additional numerical values useful for data analysis and/or providing an outcome. The term “normalized count profile” as used herein refers to a profile generated using normalized counts. Examples of methods that can be used to generate normalized counts and normalized count profiles are described herein. As noted, mapped sequence reads that have been counted can be normalized with respect to test sample counts or reference sample counts. In some embodiments, a normalized count profile can be presented as a plot.

Described in greater detail hereafter are non-limiting examples of processing steps and normalization methods that can be utilized, such as normalizing to a window (static or sliding), weighting, determining bias relationship, LOESS normalization, principal component normalization, hybrid normalization, generating a profile and performing a comparison.

### d) Normalizing to a Window (Static or Sliding)

In certain embodiments, a processing step comprises normalizing to a static window, and in some embodiments, a processing step comprises normalizing to a moving or sliding window. The term “window” as used herein refers to one or more portions chosen for analysis, and sometimes is used as a reference for comparison (e.g., used for normalization and/or other mathematical or statistical manipulation). The term “normalizing to a static window” as used herein refers to a normalization process using one or more portions selected for comparison between a test subject and reference subject data set. In some embodiments the selected portions are utilized to generate a profile. A static window generally includes a predetermined set of portions that do not change during manipulations and/or analysis. The terms “normalizing to a moving window” and “normalizing to a sliding window” as used herein refer to normalizations performed to portions localized to the genomic region (e.g., immediate surrounding portions, adjacent portion or sections, and the like) of a selected test portion, where one or more selected test portions are normalized to portions immediately surrounding the selected test portion. In certain embodiments, the selected portions are utilized to generate a profile. A sliding or moving window normalization often includes repeatedly moving or sliding to an adjacent test portion, and normalizing the newly selected test portion to portions immediately surrounding or adjacent to the newly selected test portion, where adjacent windows have one or more portions in common. In certain embodiments, a plurality of selected test portions and/or chromosomes can be analyzed by a sliding window process.

In some embodiments, normalizing to a sliding or moving window can generate one or more values, where each value represents normalization to a different set of reference portions selected from different regions of a genome (e.g., chromosome). In certain embodiments, the one or more values generated are cumulative sums (e.g., a numerical estimate of the integral of the normalized count profile over the selected portion, domain (e.g., part of chromosome), or chromosome). The values generated by the sliding or moving window process can be used to generate a profile and facilitate arriving at an outcome. In some embodiments, cumulative sums of one or more portions can be displayed as a function of genomic position. Moving or sliding window analysis sometimes is used to analyze a genome for the presence or absence of microdeletions and/or microduplications. In certain embodiments, displaying cumulative sums of one or more portions is used to identify the presence or absence of regions of copy number alteration (e.g., microdeletion, microduplication).

### e) Weighting

In some embodiments, a processing step comprises a weighting. The terms “weighted,” “weighting” or “weight function” or grammatical derivatives or equivalents thereof, as used herein, refer to a mathematical manipulation of a portion or all of a data set sometimes utilized to alter the influence of certain data set features or variables with respect to other data set features or variables (e.g., increase or decrease the significance and/or contribution of data contained in one or more portions or portions of a reference genome, based on the quality or usefulness of the data in the selected portion or portions of a reference genome). A weighting function can be used to increase the influence of data with a relatively small measurement variance, and/or to decrease the influence of data with a relatively large measurement variance, in some embodiments. For example, portions of a reference genome with underrepresented or low quality sequence data can be “down weighted” to minimize the influence on a data set, whereas selected portions of a reference genome can be “up weighted” to increase the influence on a data set. A non-limiting example of a weighting function is [1/(standard deviation) 2]. Weighting portions sometimes removes portion dependencies. In some embodiments one or more portions are weighted by an eigen function (e.g., an eigenfunction). In some embodiments an eigen function comprises replacing portions with orthogonal eigen-portions. A weighting step sometimes is performed in a manner substantially similar to a normalizing step. In some embodiments, a data set is adjusted (e.g., divided, multiplied, added, subtracted) by a predetermined variable (e.g., weighting variable). In some embodiments, a data set is divided by a predetermined variable (e.g., weighting variable). A predetermined variable (e.g., minimized target function, Phi) often is selected to weigh different parts of a data set differently (e.g., increase the influence of certain data types while decreasing the influence of other data types).

### f) Bias Relationships

In some embodiments, a processing step comprises determining a bias relationship. For example, one or more relationships may be generated between local genome bias estimates and bias frequencies. The term “relationship” as use herein refers to a mathematical and/or a graphical relationship between two or more variables or values. A relationship can be generated by a suitable mathematical and/or graphical process. Non-limiting examples of a relationship include a mathematical and/or graphical representation of a function, a correlation, a distribution, a linear or non-linear equation, a line, a regression, a fitted regression, the like or a combination thereof. Sometimes a relationship comprises a fitted relationship. In some embodiments a fitted relationship comprises a fitted regression. Sometimes a relationship comprises two or more variables or values that are weighted. In some embodiments a relationship comprise a fitted regression where one or more variables or values of the relationship a weighted. Sometimes a regression is fitted in a weighted fashion. Sometimes a regression is fitted without weighting. In certain embodiments, generating a relationship comprises plotting or graphing.

In certain embodiments, a relationship is generated between GC densities and GC density frequencies. In some embodiments generating a relationship between (i) GC densities and (ii) GC density frequencies for a sample provides a sample GC density relationship. In some embodiments generating a relationship between (i) GC densities and (ii) GC density frequencies for a reference provides a reference GC density relationship. In some embodiments, where local genome bias estimates are GC densities, a sample bias relationship is a sample GC density relationship and a reference bias relationship is a reference GC density relationship. GC densities of a reference GC density relationship and/or a sample GC density relationship are often representations (e.g., mathematical or quantitative representation) of local GC content.

In some embodiments a relationship between local genome bias estimates and bias frequencies comprises a distribution. In some embodiments a relationship between local genome bias estimates and bias frequencies comprises a fitted relationship (e.g., a fitted regression). In some embodiments a relationship between local genome bias estimates and bias frequencies comprises a fitted linear or non-linear regression (e.g., a polynomial regression). In certain embodiments a relationship between local genome bias estimates and bias frequencies comprises a weighted relationship where local genome bias estimates and/or bias frequencies are weighted by a suitable process. In some embodiments a weighted fitted relationship (e.g., a weighted fitting) can be obtained by a process comprising a quantile regression, parameterized distributions or an empirical distribution with interpolation. In certain embodiments a relationship between local genome bias estimates and bias frequencies for a test sample, a reference or part thereof, comprises a polynomial regression where local genome bias estimates are weighted. In some embodiments a weighed fitted model comprises weighting values of a distribution. Values of a distribution can be weighted by a suitable process. In some embodiments, values located near tails of a distribution are provided less weight than values closer to the median of the distribution. For example, for a distribution between local genome bias estimates (e.g., GC densities) and bias frequencies (e.g., GC density frequencies), a weight is determined according to the bias frequency for a given local genome bias estimate, where local genome bias estimates comprising bias frequencies closer to the mean of a distribution are provided greater weight than local genome bias estimates comprising bias frequencies further from the mean.

In some embodiments, a processing step comprises normalizing sequence read counts by comparing local genome bias estimates of sequence reads of a test sample to local genome bias estimates of a reference (e.g., a reference genome, or part thereof). In some embodiments, counts of sequence reads are normalized by comparing bias frequencies of local genome bias estimates of a test sample to bias frequencies of local genome bias estimates of a reference. In some embodiments counts of sequence reads are normalized by comparing a sample bias relationship and a reference bias relationship, thereby generating a comparison.

Counts of sequence reads may be normalized according to a comparison of two or more relationships. In certain embodiments two or more relationships are compared thereby providing a comparison that is used for reducing local bias in sequence reads (e.g., normalizing counts). Two or more relationships can be compared by a suitable method. In some embodiments a comparison comprises adding, subtracting, multiplying and/or dividing a first relationship from a second relationship. In certain embodiments comparing two or more relationships comprises a use of a suitable linear regression and/or a non-linear regression. In certain embodiments comparing two or more relationships comprises a suitable polynomial regression (e.g., a 3rd order polynomial regression). In some embodiments a comparison comprises adding, subtracting, multiplying and/or dividing a first regression from a second regression. In some embodiments two or more relationships are compared by a process comprising an inferential framework of multiple regressions. In some embodiments two or more relationships are compared by a process comprising a suitable multivariate analysis. In some embodiments two or more relationships are compared by a process comprising a basis function (e.g., a blending function, e.g., polynomial bases, Fourier bases, or the like), splines, a radial basis function and/or wavelets.

In certain embodiments a distribution of local genome bias estimates comprising bias frequencies for a test sample and a reference is compared by a process comprising a polynomial regression where local genome bias estimates are weighted. In some embodiments a polynomial regression is generated between (i) ratios, each of which ratios comprises bias frequencies of local genome bias estimates of a reference and bias frequencies of local genome bias estimates of a sample and (ii) local genome bias estimates. In some embodiments a polynomial regression is generated between (i) a ratio of bias frequencies of local genome bias estimates of a reference to bias frequencies of local genome bias estimates of a sample and (ii) local genome bias estimates. In some embodiments a comparison of a distribution of local genome bias estimates for reads of a test sample and a reference comprises determining a log ratio (e.g., a log 2 ratio) of bias frequencies of local genome bias estimates for the reference and the sample. In some embodiments a comparison of a distribution of local genome bias estimates comprises dividing a log ratio (e.g., a log 2 ratio) of bias frequencies of local genome bias estimates for the reference by a log ratio (e.g., a log 2 ratio) of bias frequencies of local genome bias estimates for the sample.

Normalizing counts according to a comparison typically adjusts some counts and not others. Normalizing counts sometimes adjusts all counts and sometimes does not adjust any counts of sequence reads. A count for a sequence read sometimes is normalized by a process that comprises determining a weighting factor and sometimes the process does not include directly generating and utilizing a weighting factor. Normalizing counts according to a comparison sometimes comprises determining a weighting factor for each count of a sequence read. A weighting factor is often specific to a sequence read and is applied to a count of a specific sequence read. A weighting factor is often determined according to a comparison of two or more bias relationships (e.g., a sample bias relationship compared to a reference bias relationship). A normalized count is often determined by adjusting a count value according to a weighting factor. Adjusting a count according to a weighting factor sometimes includes adding, subtracting, multiplying and/or dividing a count for a sequence read by a weighting factor. A weighting factor and/or a normalized count sometimes are determined from a regression (e.g., a regression line). A normalized count is sometimes obtained directly from a regression line (e.g., a fitted regression line) resulting from a comparison between bias frequencies of local genome bias estimates of a reference (e.g., a reference genome) and a test sample. In some embodiments each count of a read of a sample is provided a normalized count value according to a comparison of (i) bias frequencies of a local genome bias estimates of reads compared to (ii) bias frequencies of a local genome bias estimates of a reference. In certain embodiments, counts of sequence reads obtained for a sample are normalized and bias in the sequence reads is reduced.

### g) LOESS Normalization

In some embodiments, a processing step comprises a LOESS normalization. LOESS is a regression modeling method known in the art that combines multiple regression models in a k-nearest-neighbor-based meta-model. LOESS is sometimes referred to as a locally weighted polynomial regression. GC LOESS, in some embodiments, applies a LOESS model to the relationship between fragment count (e.g., sequence reads, counts) and GC composition for portions of a reference genome. Plotting a smooth curve through a set of data points using LOESS is sometimes called a LOESS curve, particularly when each smoothed value is given by a weighted quadratic least squares regression over the span of values of the y-axis scattergram criterion variable. For each point in a data set, the LOESS method fits a low-degree polynomial to a subset of the data, with explanatory variable values near the point whose response is being estimated. The polynomial is fitted using weighted least squares, giving more weight to points near the point whose response is being estimated and less weight to points further away. The value of the regression function for a point is then obtained by evaluating the local polynomial using the explanatory variable values for that data point. The LOESS fit is sometimes considered complete after regression function values have been computed for each of the data points. Many of the details of this method, such as the degree of the polynomial model and the weights, are flexible.

### h) Principal Component Analysis

In some embodiments, a processing step comprises a principal component analysis (PCA). In some embodiments, sequence read counts (e.g., sequence read counts of a test sample) is adjusted according to a principal component analysis (PCA). In some embodiments a read density profile (e.g., a read density profile of a test sample) is adjusted according to a principal component analysis (PCA). A read density profile of one or more reference samples and/or a read density profile of a test subject can be adjusted according to a PCA. Removing bias from a read density profile by a PCA related process is sometimes referred to herein as adjusting a profile. A PCA can be performed by a suitable PCA method, or a variation thereof. Non-limiting examples of a PCA method include a canonical correlation analysis (CCA), a Karhunen-Loève transform (KLT), a Hotelling transform, a proper orthogonal decomposition (POD), a singular value decomposition (SVD) of X, an eigenvalue decomposition (EVD) of XTX, a factor analysis, an Eckart-Young theorem, a Schmidt-Mirsky theorem, empirical orthogonal functions (EOF), an empirical eigenfunction decomposition, an empirical component analysis, quasiharmonic modes, a spectral decomposition, an empirical modal analysis, the like, variations or combinations thereof. A PCA often identifies and/or adjusts for one or more biases in a read density profile. A bias identified and/or adjusted for by a PCA is sometimes referred to herein as a principal component. In some embodiments one or more biases can be removed by adjusting a read density profile according to one or more principal component using a suitable method. A read density profile can be adjusted by adding, subtracting, multiplying and/or dividing one or more principal components from a read density profile. In some embodiments, one or more biases can be removed from a read density profile by subtracting one or more principal components from a read density profile. Although bias in a read density profile is often identified and/or quantitated by a PCA of a profile, principal components are often subtracted from a profile at the level of read densities. A PCA often identifies one or more principal components. In some embodiments a PCA identifies a 1st, 2nd, 3rd, 4th, 5th, 6th, 7th, 8th, 9th, and a 10th or more principal components. In certain embodiments, 1, 2, 3, 4, 5, 6, 7, 8, 9, 10 or more principal components are used to adjust a profile. In certain embodiments, 5 principal components are used to adjust a profile. Often, principal components are used to adjust a profile in the order of appearance in a PCA. For example, where three principal components are subtracted from a read density profile, a 1st, 2nd and 3rd principal component are used. Sometimes a bias identified by a principal component comprises a feature of a profile that is not used to adjust a profile. For example, a PCA may identify a copy number alteration (e.g., an aneuploidy, microduplication, microdeletion, deletion, translocation, insertion) and/or a sex difference as a principal component. Thus, in some embodiments, one or more principal components are not used to adjust a profile. For example, sometimes a 1st, 2nd and 4th principal component are used to adjust a profile where a 3rd principal component is not used to adjust a profile.

A principal component can be obtained from a PCA using any suitable sample or reference. In some embodiments principal components are obtained from a test sample (e.g., a test subject). In some embodiments principal components are obtained from one or more references (e.g., reference samples, reference sequences, a reference set). In certain instances, a PCA is performed on a median read density profile obtained from a training set comprising multiple samples resulting in the identification of a 1st principal component and a 2nd principal component. In some embodiments, principal components are obtained from a set of subjects devoid of a copy number alteration in question. In some embodiments, principal components are obtained from a set of known euploids. Principal components are often identified according to a PCA performed using one or more read density profiles of a reference (e.g., a training set). One or more principal components obtained from a reference are often subtracted from a read density profile of a test subject thereby providing an adjusted profile.

### i) Hybrid Normalization

In some embodiments, a processing step comprises a hybrid normalization method. A hybrid normalization method may reduce bias (e.g., GC bias), in certain instances. A hybrid normalization, in some embodiments, comprises (i) an analysis of a relationship of two variables (e.g., counts and GC content) and (ii) selection and application of a normalization method according to the analysis. A hybrid normalization, in certain embodiments, comprises (i) a regression (e.g., a regression analysis) and (ii) selection and application of a normalization method according to the regression. In some embodiments counts obtained for a first sample (e.g., a first set of samples) are normalized by a different method than counts obtained from another sample (e.g., a second set of samples). In some embodiments counts obtained for a first sample (e.g., a first set of samples) are normalized by a first normalization method and counts obtained from a second sample (e.g., a second set of samples) are normalized by a second normalization method. For example, in certain embodiments a first normalization method comprises use of a linear regression and a second normalization method comprises use of a non-linear regression (e.g., a LOESS, GC-LOESS, LOWESS regression, LOESS smoothing).

In some embodiments a hybrid normalization method is used to normalize sequence reads mapped to portions of a genome or chromosome (e.g., counts, mapped counts, mapped reads). In certain embodiments raw counts are normalized and in some embodiments adjusted, weighted, filtered or previously normalized counts are normalized by a hybrid normalization method. In certain embodiments, levels or Z-scores are normalized. In some embodiments counts mapped to selected portions of a genome or chromosome are normalized by a hybrid normalization approach. Counts can refer to a suitable measure of sequence reads mapped to portions of a genome, non-limiting examples of which include raw counts (e.g., unprocessed counts), normalized counts (e.g., normalized by LOESS, principal component, or a suitable method), portion levels (e.g., average levels, mean levels, median levels, or the like), Z-scores, the like, or combinations thereof. The counts can be raw counts or processed counts from one or more samples (e.g., a test sample, a sample from a pregnant female). In some embodiments counts are obtained from one or more samples obtained from one or more subjects.

In some embodiments a normalization method (e.g., the type of normalization method) is selected according to a regression (e.g., a regression analysis) and/or a correlation coefficient. A regression analysis refers to a statistical technique for estimating a relationship among variables (e.g., counts and GC content). In some embodiments a regression is generated according to counts and a measure of GC content for each portion of multiple portions of a reference genome.

Any suitable measure of GC content can be used, non-limiting examples of which include a measure of guanine, cytosine, adenine, thymine, purine (GC), or pyrimidine (AT or ATU) content, melting temperature (Tm) (e.g., denaturation temperature, annealing temperature, hybridization temperature), a measure of free energy, the like or combinations thereof. A measure of guanine (G), cytosine (C), adenine (A), thymine (T), purine (GC), or pyrimidine (AT or ATU) content can be expressed as a ratio or a percentage. In some embodiments any suitable ratio or percentage is used, non-limiting examples of which include GC/AT, GC/total nucleotide, GC/A, GC/T, AT/total nucleotide, AT/GC, AT/G, AT/C, G/A, C/A, G/T, G/A, G/AT, C/T, the like or combinations thereof. In some embodiments a measure of GC content is a ratio or percentage of GC to total nucleotide content. In some embodiments a measure of GC content is a ratio or percentage of GC to total nucleotide content for sequence reads mapped to a portion of reference genome. In certain embodiments the GC content is determined according to and/or from sequence reads mapped to each portion of a reference genome and the sequence reads are obtained from a sample. In some embodiments a measure of GC content is not determined according to and/or from sequence reads. In certain embodiments, a measure of GC content is determined for one or more samples obtained from one or more subjects.

In some embodiments generating a regression comprises generating a regression analysis or a correlation analysis. A suitable regression can be used, non-limiting examples of which include a regression analysis, (e.g., a linear regression analysis), a goodness of fit analysis, a Pearson's correlation analysis, a rank correlation, a fraction of variance unexplained, Nash-Sutcliffe model efficiency analysis, regression model validation, proportional reduction in loss, root mean square deviation, the like or a combination thereof. In some embodiments a regression line is generated. In certain embodiments generating a regression comprises generating a linear regression. In certain embodiments generating a regression comprises generating a non-linear regression (e.g., a LOESS regression, an LOWESS regression).

In some embodiments a regression determines the presence or absence of a correlation (e.g., a linear correlation), for example between counts and a measure of GC content. In some embodiments a regression (e.g., a linear regression) is generated and a correlation coefficient is determined. In some embodiments a suitable correlation coefficient is determined, non-limiting examples of which include a coefficient of determination, an R2 value, a Pearson's correlation coefficient, or the like.

In some embodiments goodness of fit is determined for a regression (e.g., a regression analysis, a linear regression). Goodness of fit sometimes is determined by visual or mathematical analysis. An assessment sometimes includes determining whether the goodness of fit is greater for a non-linear regression or for a linear regression. In some embodiments a correlation coefficient is a measure of a goodness of fit. In some embodiments an assessment of a goodness of fit for a regression is determined according to a correlation coefficient and/or a correlation coefficient cutoff value. In some embodiments an assessment of a goodness of fit comprises comparing a correlation coefficient to a correlation coefficient cutoff value. In some embodiments an assessment of a goodness of fit for a regression is indicative of a linear regression. For example, in certain embodiments, a goodness of fit is greater for a linear regression than for a non-linear regression and the assessment of the goodness of fit is indicative of a linear regression. In some embodiments an assessment is indicative of a linear regression and a linear regression is used to normalize the counts. In some embodiments an assessment of a goodness of fit for a regression is indicative of a non-linear regression. For example, in certain embodiments, a goodness of fit is greater for a non-linear regression than for a linear regression and the assessment of the goodness of fit is indicative of a non-linear regression. In some embodiments an assessment is indicative of a non-linear regression and a non-linear regression is used to normalize the counts.

In some embodiments an assessment of a goodness of fit is indicative of a linear regression when a correlation coefficient is equal to or greater than a correlation coefficient cutoff. In some embodiments an assessment of a goodness of fit is indicative of a non-linear regression when a correlation coefficient is less than a correlation coefficient cutoff. In some embodiments a correlation coefficient cutoff is pre-determined. In some embodiments a correlation coefficient cutoff is about 0.5 or greater, about 0.55 or greater, about 0.6 or greater, about 0.65 or greater, about 0.7 or greater, about 0.75 or greater, about 0.8 or greater, about 0.85 or greater, about 0.9 or greater, or about 0.95 or greater.

In some embodiments a specific type of regression is selected (e.g., a linear or non-linear regression) and, after the regression is generated, counts are normalized by subtracting the regression from the counts. In some embodiments subtracting a regression from the counts provides normalized counts with reduced bias (e.g., GC bias). In some embodiments a linear regression is subtracted from the counts. In some embodiments a non-linear regression (e.g., a LOESS, GC-LOESS, LOWESS regression) is subtracted from the counts. Any suitable method can be used to subtract a regression line from the counts. For example, if counts x are derived from portion i (e.g., a portion i) comprising a GC content of 0.5 and a regression line determines counts y at a GC content of 0.5, then x-y=normalized counts for portion i. In some embodiments counts are normalized prior to and/or after subtracting a regression. In some embodiments, counts normalized by a hybrid normalization approach are used to generate levels, Z-scores, levels and/or profiles of a genome or a part thereof. In certain embodiments, counts normalized by a hybrid normalization approach are analyzed by methods described herein to determine the presence or absence of a genetic variation or genetic alteration (e.g., copy number alteration).

In some embodiments a hybrid normalization method comprises filtering or weighting one or more portions before or after normalization. A suitable method of filtering portions, including methods of filtering portions (e.g., portions of a reference genome) described herein can be used. In some embodiments, portions (e.g., portions of a reference genome) are filtered prior to applying a hybrid normalization method. In some embodiments, only counts of sequencing reads mapped to selected portions (e.g., portions selected according to count variability) are normalized by a hybrid normalization. In some embodiments counts of sequencing reads mapped to filtered portions of a reference genome (e.g., portions filtered according to count variability) are removed prior to utilizing a hybrid normalization method. In some embodiments a hybrid normalization method comprises selecting or filtering portions (e.g., portions of a reference genome) according to a suitable method (e.g., a method described herein). In some embodiments a hybrid normalization method comprises selecting or filtering portions (e.g., portions of a reference genome) according to an uncertainty value for counts mapped to each of the portions for multiple test samples. In some embodiments a hybrid normalization method comprises selecting or filtering portions (e.g., portions of a reference genome) according to count variability. In some embodiments a hybrid normalization method comprises selecting or filtering portions (e.g., portions of a reference genome) according to GC content, repetitive elements, repetitive sequences, introns, exons, the like or a combination thereof.

### j) Profiles

In some embodiments, a processing step comprises generating one or more profiles (e.g., profile plot) from various aspects of a data set or derivation thereof (e.g., product of one or more mathematical and/or statistical data processing steps known in the art and/or described herein). The term “profile” as used herein refers to a product of a mathematical and/or statistical manipulation of data that can facilitate identification of patterns and/or correlations in large quantities of data. A “profile” often includes values resulting from one or more manipulations of data or data sets, based on one or more criteria. A profile often includes multiple data points. Any suitable number of data points may be included in a profile depending on the nature and/or complexity of a data set. In certain embodiments, profiles may include 2 or more data points, 3 or more data points, 5 or more data points, 10 or more data points, 24 or more data points, 25 or more data points, 50 or more data points, 100 or more data points, 500 or more data points, 1000 or more data points, 5000 or more data points, 10,000 or more data points, or 100,000 or more data points.

In some embodiments, a profile is representative of the entirety of a data set, and in certain embodiments, a profile is representative of a part or subset of a data set. That is, a profile sometimes includes or is generated from data points representative of data that has not been filtered to remove any data, and sometimes a profile includes or is generated from data points representative of data that has been filtered to remove unwanted data. In some embodiments, a data point in a profile represents the results of data manipulation for a portion. In certain embodiments, a data point in a profile includes results of data manipulation for groups of portions. In some embodiments, groups of portions may be adjacent to one another, and in certain embodiments, groups of portions may be from different parts of a chromosome or genome.

Data points in a profile derived from a data set can be representative of any suitable data categorization. Non-limiting examples of categories into which data can be grouped to generate profile data points include: portions based on size, portions based on sequence features (e.g., GC content, AT content, position on a chromosome (e.g., short arm, long arm, centromere, telomere), and the like), levels of expression, chromosome, the like or combinations thereof. In some embodiments, a profile may be generated from data points obtained from another profile (e.g., normalized data profile renormalized to a different normalizing value to generate a renormalized data profile). In certain embodiments, a profile generated from data points obtained from another profile reduces the number of data points and/or complexity of the data set. Reducing the number of data points and/or complexity of a data set often facilitates interpretation of data and/or facilitates providing an outcome.

A profile (e.g., a genomic profile, a chromosome profile, a profile of a part of a chromosome) often is a collection of normalized or non-normalized counts for two or more portions. A profile often includes at least one level, and often comprises two or more levels (e.g., a profile often has multiple levels). A level generally is for a set of portions having about the same counts or normalized counts. Levels are described in greater detail herein. In certain embodiments, a profile comprises one or more portions, which portions can be weighted, removed, filtered, normalized, adjusted, averaged, derived as a mean, added, subtracted, processed or transformed by any combination thereof. A profile often comprises normalized counts mapped to portions defining two or more levels, where the counts are further normalized according to one of the levels by a suitable method. Often counts of a profile (e.g., a profile level) are associated with an uncertainty value.

A profile comprising one or more levels is sometimes padded (e.g., hole padding). Padding (e.g., hole padding) refers to a process of identifying and adjusting levels in a profile that are due to copy number alterations (e.g., microduplications or microdeletions in a patient's genome, maternal microduplications or microdeletions). In some embodiments, levels are padded that are due to microduplications or microdeletions in a tumor or a fetus. Microduplications or microdeletions in a profile can, in some embodiments, artificially raise or lower the overall level of a profile (e.g., a profile of a chromosome) leading to false positive or false negative determinations of a chromosome aneuploidy (e.g., a trisomy). In some embodiments, levels in a profile that are due to microduplications and/or deletions are identified and adjusted (e.g., padded and/or removed) by a process sometimes referred to as padding or hole padding.

A profile comprising one or more levels can include a first level and a second level. In some embodiments a first level is different (e.g., significantly different) than a second level. In some embodiments a first level comprises a first set of portions, a second level comprises a second set of portions and the first set of portions is not a subset of the second set of portions. In certain embodiments, a first set of portions is different than a second set of portions from which a first and second level are determined. In some embodiments a profile can have multiple first levels that are different (e.g., significantly different, e.g., have a significantly different value) than a second level within the profile. In some embodiments a profile comprises one or more first levels that are significantly different than a second level within the profile and one or more of the first levels are adjusted. In some embodiments a first level within a profile is removed from the profile or adjusted (e.g., padded). A profile can comprise multiple levels that include one or more first levels significantly different than one or more second levels and often the majority of levels in a profile are second levels, which second levels are about equal to one another. In some embodiments greater than 50%, greater than 60%, greater than 70%, greater than 80%, greater than 90% or greater than 95% of the levels in a profile are second levels.

A profile sometimes is displayed as a plot. For example, one or more levels representing counts (e.g., normalized counts) of portions can be plotted and visualized. Non-limiting examples of profile plots that can be generated include raw count (e.g., raw count profile or raw profile), normalized count, portion-weighted, z-score, p-value, area ratio versus fitted ploidy, median level versus ratio between fitted and measured minority species fraction, principal components, the like, or combinations thereof. Profile plots allow visualization of the manipulated data, in some embodiments. In certain embodiments, a profile plot can be utilized to provide an outcome (e.g., area ratio versus fitted ploidy, median level versus ratio between fitted and measured minority species fraction, principal components). The terms “raw count profile plot” or “raw profile plot” as used herein refer to a plot of counts in each portion in a region normalized to total counts in a region (e.g., genome, portion, chromosome, chromosome portions of a reference genome or a part of a chromosome). In some embodiments, a profile can be generated using a static window process, and in certain embodiments, a profile can be generated using a sliding window process.

A profile generated for a test subject sometimes is compared to a profile generated for one or more reference subjects, to facilitate interpretation of mathematical and/or statistical manipulations of a data set and/or to provide an outcome. In some embodiments, a profile is generated based on one or more starting assumptions, e.g., assumptions described herein. In certain embodiments, a test profile often centers around a predetermined value representative of the absence of a copy number alteration, and often deviates from a predetermined value in areas corresponding to the genomic location in which the copy number alteration is located in the test subject, if the test subject possessed the copy number alteration. In test subjects at risk for, or suffering from a medical condition associated with a copy number alteration, the numerical value for a selected portion is expected to vary significantly from the predetermined value for non-affected genomic locations. Depending on starting assumptions (e.g., fixed ploidy or optimized ploidy, fixed fraction of cancer cell nucleic acid or optimized fraction of cancer cell nucleic acid, fixed fetal fraction or optimized fetal fraction, or combinations thereof) the predetermined threshold or cutoff value or threshold range of values indicative of the presence or absence of a copy number alteration can vary while still providing an outcome useful for determining the presence or absence of a copy number alteration. In some embodiments, a profile is indicative of and/or representative of a phenotype.

In some embodiments, the use of one or more reference samples that are substantially free of a copy number alteration in question can be used to generate a reference count profile (e.g., a reference median count profile), which may result in a predetermined value representative of the absence of the copy number alteration, and often deviates from a predetermined value in areas corresponding to the genomic location in which the copy number alteration is located in the test subject, if the test subject possessed the copy number alteration. In test subjects at risk for, or suffering from a medical condition associated with a copy number alteration, the numerical value for the selected portion or sections is expected to vary significantly from the predetermined value for non-affected genomic locations. In certain embodiments, the use of one or more reference samples known to carry the copy number alteration in question can be used to generate a reference count profile (a reference median count profile), which may result in a predetermined value representative of the presence of the copy number alteration, and often deviates from a predetermined value in areas corresponding to the genomic location in which a test subject does not carry the copy number alteration. In test subjects not at risk for, or suffering from a medical condition associated with a copy number alteration, the numerical value for the selected portion or sections is expected to vary significantly from the predetermined value for affected genomic locations.

By way of a non-limiting example, normalized sample and/or reference count profiles can be obtained from raw sequence read data by (a) calculating reference median counts for selected chromosomes, portions or parts thereof from a set of references known not to carry a copy number alteration, (b) removal of uninformative portions from the reference sample raw counts (e.g., filtering); (c) normalizing the reference counts for all remaining portions of a reference genome to the total residual number of counts (e.g., sum of remaining counts after removal of uninformative portions of a reference genome) for the reference sample selected chromosome or selected genomic location, thereby generating a normalized reference subject profile; (d) removing the corresponding portions from the test subject sample; and (e) normalizing the remaining test subject counts for one or more selected genomic locations to the sum of the residual reference median counts for the chromosome or chromosomes containing the selected genomic locations, thereby generating a normalized test subject profile. In certain embodiments, an additional normalizing step with respect to the entire genome, reduced by the filtered portions in (b), can be included between (c) and (d).

In some embodiments a read density profile is determined. In some embodiments a read density profile comprises at least one read density, and often comprises two or more read densities (e.g., a read density profile often comprises multiple read densities). In some embodiments, a read density profile comprises a suitable quantitative value (e.g., a mean, a median, a Z-score, or the like). A read density profile often comprises values resulting from one or more read densities. A read density profile sometimes comprises values resulting from one or more manipulations of read densities based on one or more adjustments (e.g., normalizations). In some embodiments a read density profile comprises unmanipulated read densities. In some embodiments, one or more read density profiles are generated from various aspects of a data set comprising read densities, or a derivation thereof (e.g., product of one or more mathematical and/or statistical data processing steps known in the art and/or described herein). In certain embodiments, a read density profile comprises normalized read densities. In some embodiments a read density profile comprises adjusted read densities. In certain embodiments a read density profile comprises raw read densities (e.g., unmanipulated, not adjusted or normalized), normalized read densities, weighted read densities, read densities of filtered portions, z-scores of read densities, p-values of read densities, integral values of read densities (e.g., area under the curve), average, mean or median read densities, principal components, the like, or combinations thereof. Often read densities of a read density profile and/or a read density profile is associated with a measure of uncertainty (e.g., a MAD). In certain embodiments, a read density profile comprises a distribution of median read densities. In some embodiments a read density profile comprises a relationship (e.g., a fitted relationship, a regression, or the like) of a plurality of read densities. For example, sometimes a read density profile comprises a relationship between read densities (e.g., read densities value) and genomic locations (e.g., portions, portion locations). In some embodiments, a read density profile is generated using a static window process, and in certain embodiments, a read density profile is generated using a sliding window process. In some embodiments a read density profile is sometimes printed and/or displayed (e.g., displayed as a visual representation, e.g., a plot or a graph).

In some embodiments, a read density profile corresponds to a set of portions (e.g., a set of portions of a reference genome, a set of portions of a chromosome or a subset of portions of a part of a chromosome). In some embodiments a read density profile comprises read densities and/or counts associated with a collection (e.g., a set, a subset) of portions. In some embodiments, a read density profile is determined for read densities of portions that are contiguous. In some embodiments, contiguous portions comprise gaps comprising regions of a reference sequence and/or sequence reads that are not included in a density profile (e.g., portions removed by a filtering). Sometimes portions (e.g., a set of portions) that are contiguous represent neighboring regions of a genome or neighboring regions of a chromosome or gene. For example, two or more contiguous portions, when aligned by merging the portions end to end, can represent a sequence assembly of a DNA sequence longer than each portion. For example, two or more contiguous portions can represent an intact genome, chromosome, gene, intron, exon or part thereof. Sometimes a read density profile is determined from a collection (e.g., a set, a subset) of contiguous portions and/or non-contiguous portions. In some cases, a read density profile comprises one or more portions, which portions can be weighted, removed, filtered, normalized, adjusted, averaged, derived as a mean, added, subtracted, processed or transformed by any combination thereof.

A read density profile is often determined for a sample and/or a reference (e.g., a reference sample). A read density profile is sometimes generated for an entire genome, one or more chromosomes, or for a part of a genome or a chromosome. In some embodiments, one or more read density profiles are determined for a genome or part thereof. In some embodiments, a read density profile is representative of the entirety of a set of read densities of a sample, and in certain embodiments, a read density profile is representative of a part or subset of read densities of a sample. That is, sometimes a read density profile comprises or is generated from read densities representative of data that has not been filtered to remove any data, and sometimes a read density profile includes or is generated from data points representative of data that has been filtered to remove unwanted data.

In some embodiments a read density profile is determined for a reference (e.g., a reference sample, a training set). A read density profile for a reference is sometimes referred to herein as a reference profile. In some embodiments a reference profile comprises a read densities obtained from one or more references (e.g., reference sequences, reference samples). In some embodiments a reference profile comprises read densities determined for one or more (e.g., a set of) known euploid samples. In some embodiments a reference profile comprises read densities of filtered portions. In some embodiments a reference profile comprises read densities adjusted according to the one or more principal components.

### 3) Bioinformatic Analysis

At step 130, the processed sequence read data is analyzed using one or more bioinformatics subsystems. In certain instances, the one or more bioinformatics subsystems comprises one or more machine-learning models. For example, the processed sequence read data may be analyzed using one or more bioinformatics subsystems (e.g., a Chromosomal Aberration Decision Tree (CADET)) to detect a genome event (e.g., a presence or absence of a genetic variation).

### a) Performing a Comparison

In some embodiments, a processing step comprises preforming a comparison (e.g., comparing a test profile to a reference profile). Two or more data sets, two or more relationships and/or two or more profiles can be compared by a suitable method. Non-limiting examples of statistical methods suitable for comparing data sets, relationships and/or profiles include Behrens-Fisher approach, bootstrapping, Fisher's method for combining independent tests of significance, Neyman-Pearson testing, confirmatory data analysis, exploratory data analysis, exact test, F-test, Z-test, T-test, calculating and/or comparing a measure of uncertainty, a null hypothesis, counternulls and the like, a chi-square test, omnibus test, calculating and/or comparing level of significance (e.g., statistical significance), a meta-analysis, a multivariate analysis, a regression, simple linear regression, robust linear regression, the like or combinations of the foregoing. In certain embodiments comparing two or more data sets, relationships and/or profiles comprises determining and/or comparing a measure of uncertainty. A “measure of uncertainty” as used herein refers to a measure of significance (e.g., statistical significance), a measure of error, a measure of variance, a measure of confidence, the like or a combination thereof. A measure of uncertainty can be a value (e.g., a threshold) or a range of values (e.g., an interval, a confidence interval, a Bayesian confidence interval, a threshold range). Non-limiting examples of a measure of uncertainty include p-values, a suitable measure of deviation (e.g., standard deviation, sigma, absolute deviation, mean absolute deviation, the like), a suitable measure of error (e.g., standard error, mean squared error, root mean squared error, the like), a suitable measure of variance, a suitable standard score (e.g., standard deviations, cumulative percentages, percentile equivalents, Z-scores, T-scores, R-scores, standard nine (stanine), percent in stanine, the like), the like or combinations thereof. In some embodiments determining the level of significance comprises determining a measure of uncertainty (e.g., a p-value). In certain embodiments, two or more data sets, relationships and/or profiles can be analyzed and/or compared by utilizing multiple (e.g., 2 or more) statistical methods (e.g., least squares regression, principal component analysis, linear discriminant analysis, quadratic discriminant analysis, bagging, neural networks, support vector machine models, random forests, classification tree models, K-nearest neighbors, logistic regression and/or loss smoothing) and/or any suitable mathematical and/or statistical manipulations (e.g., referred to herein as manipulations).

In some embodiments, a processing step comprises a comparison of two or more profiles (e.g., two or more read density profiles). Comparing profiles may comprise comparing profiles generated for a selected region of a genome. For example, a test profile may be compared to a reference profile where the test and reference profiles were determined for a region of a genome (e.g., a reference genome) that is substantially the same region. Comparing profiles sometimes comprises comparing two or more subsets of portions of a profile (e.g., a read density profile). A subset of portions of a profile may represent a region of a genome (e.g., a chromosome, or region thereof). A profile (e.g., a read density profile) can comprise any amount of subsets of portions. Sometimes a profile (e.g., a read density profile) comprises two or more, three or more, four or more, or five or more subsets. In certain embodiments, a profile (e.g., a read density profile) comprises two subsets of portions where each portion represents regions of a reference genome that are adjacent. In some embodiments, a test profile can be compared to a reference profile where the test profile and reference profile both comprise a first subset of portions and a second subset of portions where the first and second subsets represent different regions of a genome. Some subsets of portions of a profile may comprise copy number alterations and other subsets of portions are sometimes substantially free of copy number alterations. Sometimes all subsets of portions of a profile (e.g., a test profile) are substantially free of a copy number alteration. Sometimes all subsets of portions of a profile (e.g., a test profile) comprise a copy number alteration. In some embodiments a test profile can comprise a first subset of portions that comprise a copy number alteration and a second subset of portions that are substantially free of a copy number alteration.

In certain embodiments, comparing two or more profiles comprises determining and/or comparing a measure of uncertainty for two or more profiles. Profiles (e.g., read density profiles) and/or associated measures of uncertainty are sometimes compared to facilitate interpretation of mathematical and/or statistical manipulations of a data set and/or to provide an outcome. A profile (e.g., a read density profile) generated for a test subject sometimes is compared to a profile (e.g., a read density profile) generated for one or more references (e.g., reference samples, reference subjects, and the like). In some embodiments, an outcome is provided by comparing a profile (e.g., a read density profile) from a test subject to a profile (e.g., a read density profile) from a reference for a chromosome, portions or parts thereof, where a reference profile is obtained from a set of reference subjects known not to possess a copy number alteration (e.g., a reference). In some embodiments an outcome is provided by comparing a profile (e.g., a read density profile) from a test subject to a profile (e.g., a read density profile) from a reference for a chromosome, portions or parts thereof, where a reference profile is obtained from a set of reference subjects known to possess a specific copy number alteration (e.g., a chromosome aneuploidy, a microduplication, a microdeletion).

In certain embodiments, a profile (e.g., a read density profile) of a test subject is compared to a predetermined value representative of the absence of a copy number alteration, and sometimes deviates from a predetermined value at one or more genomic locations (e.g., portions) corresponding to a genomic location in which a copy number alteration is located. For example, in test subjects (e.g., subjects at risk for, or suffering from a medical condition associated with a copy number alteration), profiles are expected to differ significantly from profiles of a reference (e.g., a reference sequence, reference subject, reference set) for selected portions when a test subject comprises a copy number alteration in question. Profiles (e.g., read density profiles) of a test subject are often substantially the same as profiles (e.g., read density profiles) of a reference (e.g., a reference sequence, reference subject, reference set) for selected portions when a test subject does not comprise a copy number alteration in question. Profiles (e.g., read density profiles) may be compared to a predetermined threshold and/or threshold range. The term “threshold” as used herein refers to any number that is calculated using a qualifying data set and serves as a limit of diagnosis of a copy number alteration (e.g., an aneuploidy, a microduplication, a microdeletion, and the like). In certain embodiments a threshold is exceeded by results obtained by methods described herein and a subject is diagnosed with a copy number alteration. In some embodiments, a threshold value or range of values may be calculated by mathematically and/or statistically manipulating sequence read data (e.g., from a reference and/or subject). A predetermined threshold or threshold range of values indicative of the presence or absence of a copy number alteration can vary while still providing an outcome useful for determining the presence or absence of a copy number alteration. In certain embodiments, a profile (e.g., a read density profile) comprising normalized read densities and/or normalized counts is generated to facilitate classification and/or providing an outcome. An outcome can be provided based on a plot of a profile (e.g., a read density profile) comprising normalized counts (e.g., using a plot of such a read density profile).

### b) Decision Analysis

In some embodiments, a determination of an outcome (e.g., making a call) or a determination of the presence or absence of a copy number alteration (e.g., chromosome aneuploidy, microduplication, microdeletion) is made according to a decision analysis. Certain decision analysis features are described in International Patent Application Publication No. WO 2014/190286, the entire content of which is incorporated herein by reference for all purposes. For example, a decision analysis sometimes comprises applying one or more methods that produce one or more results, an evaluation of the results, and a series of decisions based on the results, evaluations and/or the possible consequences of the decisions and terminating at some juncture of the process where a final decision is made. In some embodiments a decision analysis is a decision tree. A decision analysis, in some embodiments, comprises coordinated use of one or more processes (e.g., process steps, e.g., algorithms). A decision analysis can be performed by a person, a system, an apparatus, software (e.g., a subsystem), a computer, a processor (e.g., a microprocessor), the like or a combination thereof. In some embodiments a decision analysis comprises a method of determining the presence or absence of a copy number alteration (e.g., chromosome aneuploidy, microduplication or microdeletion) with reduced false negative and reduced false positive determinations, compared to an instance in which no decision analysis is utilized (e.g., a determination is made directly from normalized counts). In some embodiments a decision analysis comprises determining the presence or absence of a condition associated with one or more copy number alterations.

In some embodiments a decision analysis comprises generating a profile for a genome or a region of a genome (e.g., a chromosome or part thereof). A profile can be generated by any suitable method, known or described herein. In some embodiments, a decision analysis comprises a segmenting process. Segmenting can modify and/or transform a profile thereby providing one or more decomposition renderings of a profile. A profile subjected to a segmenting process often is a profile of normalized counts mapped to portions in a reference genome or part thereof. As addressed herein, raw counts mapped to the portions can be normalized by one or more suitable normalization processes (e.g., LOESS, GC-LOESS, principal component normalization, or combination thereof) to generate a profile that is segmented as part of a decision analysis. A decomposition rendering of a profile is often a transformation of a profile. A decomposition rendering of a profile is sometimes a transformation of a profile into a representation of a genome, chromosome or part thereof.

In certain embodiments, a segmenting process utilized for the segmenting locates and identifies one or more levels within a profile that are different (e.g., substantially or significantly different) than one or more other levels within a profile. A level identified in a profile according to a segmenting process that is different than another level in the profile, and has edges that are different than another level in the profile, is referred to herein as a level for a discrete segment. A segmenting process can generate, from a profile of normalized counts or levels, a decomposition rendering in which one or more discrete segments can be identified. A discrete segment generally covers fewer portions than what is segmented (e.g., chromosome, chromosomes, autosomes).

In some embodiments, segmenting locates and identifies edges of discrete segments within a profile. In certain embodiments, one or both edges of one or more discrete segments are identified. For example, a segmentation process can identify the location (e.g., genomic coordinates, e.g., portion location) of the right and/or the left edges of a discrete segment in a profile. A discrete segment often comprises two edges. For example, a discrete segment can include a left edge and a right edge. In some embodiments, depending upon the representation or view, a left edge can be a 5′-edge and a right edge can be a 3′-edge of a nucleic acid segment in a profile. In some embodiments, a left edge can be a 3′-edge and a right edge can be a 5′-edge of a nucleic acid segment in a profile. Often the edges of a profile are known prior to segmentation and therefore, in some embodiments, the edges of a profile determine which edge of a level is a 5′-edge and which edge is 3′-edge. In some embodiments one or both edges of a profile and/or discrete segment is an edge of a chromosome.

In some embodiments, the edges of a discrete segment are determined according to a decomposition rendering generated for a reference sample (e.g., a reference profile). In some embodiments a null edge height distribution is determined according to a decomposition rendering of a reference profile (e.g., a profile of a chromosome or part thereof). In certain embodiments, the edges of a discrete segment in a profile are identified when the level of the discrete segment is outside a null edge height distribution. In some embodiments, the edges of a discrete segment in a profile are identified according to a Z-score calculated according to a decomposition rendering for a reference profile.

In some instances, segmenting generates two or more discrete segments (e.g., two or more fragmented levels, two or more fragmented segments) in a profile. In some embodiments, a decomposition rendering derived from a segmenting process is over-segmented or fragmented and comprises multiple discrete segments. Sometimes discrete segments generated by segmenting are substantially different and sometimes discrete segments generated by segmenting are substantially similar. Substantially similar discrete segments (e.g., substantially similar levels) often refers to two or more adjacent discrete segments in a segmented profile each having a level that differs by less than a predetermined level of uncertainty. In some embodiments, substantially similar discrete segments are adjacent to each other and are not separated by an intervening segment. In some embodiments, substantially similar discrete segments are separated by one or more smaller segments. In some embodiments substantially similar discrete segments are separated by about 1 to about 20, about 1 to about 15, about 1 to about 10 or about 1 to about 5 portions where one or more of the intervening portions have a level significantly different than the level of each of the substantially similar discrete segments. In some embodiments, the level of substantially similar discrete segments differs by less than about 3 times, less than about 2 times, less than about 1 time or less than about 0.5 times a level of uncertainty. Substantially similar discrete segments, in some embodiments, comprise a median level that differs by less than 3 MAD (e.g., less than 3 sigma), less than 2 MAD, less than 1 MAD or less than about 0.5 MAD, where a MAD is calculated from a median level of each of the segments. Substantially different discrete segments, in some embodiments, are not adjacent or are separated by 10 or more, 15 or more or 20 or more portions. Substantially different discrete segments generally have substantially different levels. In certain embodiments, substantially different discrete segments comprises levels that differ by more than about 2.5 times, more than about 3 times, more than about 4 times, more than about 5 times, more than about 6 times a level of uncertainty. Substantially different discrete segments, in some embodiments, comprise a median level that differs by more than 2.5 MAD (e.g., more than 2.5 sigma), more than 3 MAD, more than 4 MAD, more than about 5 MAD or more than about 6 MAD, where a MAD is calculated from a median level of each of the discrete segments.

In some embodiments, a segmentation process comprises determining (e.g., calculating) a level (e.g., a quantitative value, e.g., a mean or median level), a level of uncertainty (e.g., an uncertainty value), Z-score, Z-value, p-value, the like or combinations thereof for one or more discrete segments in a profile or part thereof. In some embodiments a level (e.g., a quantitative value, e.g., a mean or median level), a level of uncertainty (e.g., an uncertainty value), Z-score, Z-value, p-value, the like or combinations thereof are determined (e.g., calculated) for a discrete segment.

Segmenting can be performed, in full or in part, by one or more decomposition generating processes. A decomposition generating process may provide, for example, a decomposition rendering of a profile. Any decomposition generating process described herein or known in the art may be used. Non-limiting examples of a decomposition generating process include circular binary segmentation (CBS) (see e.g., Olshen et al. (2004) Biostatistics 5 (4): 557-72; Venkatraman, E S, Olshen, A B (2007) Bioinformatics 23 (6): 657-63); Haar wavelet segmentation (see e.g., Haar, Alfred (1910) Mathematische Annalen 69 (3): 331-371); maximal overlap discrete wavelet transform (MODWT) (see e.g., Hsu et al. (2005) Biostatistics 6 (2): 211-226); stationary wavelet (SWT) (see e.g., Y. Wang and S. Wang (2007) International Journal of Bioinformatics Research and Applications 3 (2): 206-222); dual-tree complex wavelet transform (DTCWT) (see e.g., Nguyen et al. (2007) Proceedings of the 7th IEEE International Conference, Boston Mass., on Oct. 14-17, 2007, pages 137-144); maximum entropy segmentation, convolution with edge detection kernel, Jensen Shannon Divergence, Kullback-Leibler divergence, Binary Recursive Segmentation, a Fourier transform, the like or combinations thereof.

In some embodiments, segmenting is accomplished by a process that comprises one process or multiple sub-processes, non-limiting examples of which include a decomposition generating process, thresholding, leveling, smoothing, polishing, the like or combination thereof. Thresholding, leveling, smoothing, polishing and the like can be performed in conjunction with a decomposition generating process, for example.

In some embodiments, a decision analysis comprises identifying a candidate segment in a decomposition rendering. A candidate segment is determined as being the most significant discrete segment in a decomposition rendering. A candidate segment may be the most significant in terms of the number of portions covered by the segment and/or in terms of the absolute value of the level of normalized counts for the segment. A candidate segment sometimes is larger and sometimes substantially larger than other discrete segments in a decomposition rendering. A candidate segment can be identified by a suitable method. In some embodiments, a candidate segment is identified by an area under the curve (AUC) analysis. In certain embodiments, where a first discrete segment has a level and/or covers a number of portions substantially larger than for another discrete segment in a decomposition rendering, the first segment comprises a larger AUC. Where a level is analyzed for AUC, an absolute value of a level often is utilized (e.g., a level corresponding to normalized counts can have a negative value for a deletion and a positive value for a duplication). In certain embodiments, an AUC is determined as an absolute value of a calculated AUC (e.g., a resulting positive value). In certain embodiments, a candidate segment, once identified (e.g., by an AUC analysis or by a suitable method) and optionally after it is validated, is selected for a z-score calculation, or the like, to determine if the candidate segment represents a genetic variation or genetic alteration (e.g., an aneuploidy, microdeletion or microduplication).

In some embodiments, a decision analysis comprises a comparison. In some embodiments, a comparison comprises comparing at least two decomposition renderings. In some embodiments, a comparison comprises comparing at least two candidate segments. In certain embodiments, each of the at least two candidate segments is from a different decomposition rendering. For example, a first candidate segment can be from a first decomposition rendering and a second candidate segment can be from a second decomposition rendering. In some embodiments, a comparison comprises determining if two decomposition renderings are substantially the same or different. In some embodiments, a comparison comprises determining if two candidate segments are substantially the same or different. Two candidate segments can be determined as substantially the same or different by a suitable comparison method, non-limiting examples of which include by visual inspection, by comparing levels or Z-scores of the two candidate segments, by comparing the edges of the two candidate segments, by overlaying either the two candidate segments or their corresponding decomposition renderings, the like or combinations thereof.

### c) Transformations

As noted above, data sometimes is transformed from one form into another form. The terms “transformed,” “transformation,” and grammatical derivations or equivalents thereof, as used herein refer to an alteration of data from a physical starting material (e.g., test subject and/or reference subject sample nucleic acid) into a digital representation of the physical starting material (e.g., sequence read data), and in some embodiments includes a further transformation into one or more numerical values or graphical representations of the digital representation that can be utilized to provide an outcome. In certain embodiments, the one or more numerical values and/or graphical representations of digitally represented data can be utilized to represent the appearance of a test subject's physical genome (e.g., virtually represent or visually represent the presence or absence of a genomic insertion, duplication or deletion; represent the presence or absence of a variation in the physical amount of a sequence associated with medical conditions). A virtual representation sometimes is further transformed into one or more numerical values or graphical representations of the digital representation of the starting material. These methods can transform physical starting material into a numerical value or graphical representation, or a representation of the physical appearance of a test subject's nucleic acid.

In some embodiments, transformation of a data set facilitates providing an outcome by reducing data complexity and/or data dimensionality. Data set complexity sometimes is reduced during the process of transforming a physical starting material into a virtual representation of the starting material (e.g., sequence reads representative of physical starting material). A suitable feature or variable can be utilized to reduce data set complexity and/or dimensionality. Non-limiting examples of features that can be chosen for use as a target feature for data processing include GC content, fetal sex prediction, fragment size (e.g., length of CCF fragments, reads or a suitable representation thereof (e.g., FRS)), fragment sequence, identification of a copy number alteration, identification of chromosomal aneuploidy, identification of particular genes or proteins, identification of cancer, diseases, inherited genes/traits, chromosomal abnormalities, a biological category, a chemical category, a biochemical category, a category of genes or proteins, a gene ontology, a protein ontology, co-regulated genes, cell signaling genes, cell cycle genes, proteins pertaining to the foregoing genes, gene variants, protein variants, co-regulated genes, co-regulated proteins, amino acid sequence, nucleotide sequence, protein structure data and the like, and combinations of the foregoing. Non-limiting examples of data set complexity and/or dimensionality reduction include; reduction of a plurality of sequence reads to profile plots, reduction of a plurality of sequence reads to numerical values (e.g., normalized values, Z-scores, p-values); reduction of multiple analysis methods to probability plots or single points; principal component analysis of derived quantities; and the like or combinations thereof.

### d) Classifications

Methods described herein can provide an outcome indicative of presence or absence of genomic instability for a test sample. Methods described herein sometimes provide an outcome indicative of a genotype and/or presence or absence of a genetic variation/alteration in a genomic region for a test sample (e.g., providing an outcome determinative of the presence or absence of a genetic variation). Methods described herein sometimes provide an outcome indicative of a phenotype and/or presence or absence of a medical condition for a test sample (e.g., providing an outcome determinative of the presence or absence of a medical condition and/or phenotype). An outcome often is part of a classification process, and a classification (e.g., classification of presence or absence of genomic instability, a genotype, a phenotype, a genetic variation and/or a medical condition for a test sample) sometimes is based on and/or includes an outcome. An outcome and/or classification sometimes is based on and/or includes a result of data processing for a test sample that facilitates determining presence or absence of genomic instability, a genotype, a phenotype, a genetic variation, genetic alteration, and/or a medical condition in a classification process (e.g., a statistic value (e.g., standard score (e.g., z-score)). An outcome and/or classification sometimes includes or is based on a score determinative of, or a call of, presence or absence of genomic instability, a genotype, a phenotype, a genetic variation, genetic alteration, and/or a medical condition. In certain embodiments, an outcome and/or classification includes a conclusion that predicts and/or determines presence or absence of genomic instability, a genotype, a phenotype, a genetic variation, genetic alteration, and/or a medical condition in a classification process. In some instances, the outcome and/or classification are the output of one or more machine-learning models trained for predicting a presence or absence of genomic instability for a test sample.

A genotype and/or genetic variation often includes a gain, a loss and/or alteration of a region comprising one or more nucleotides (e.g., duplication, deletion, fusion, insertion, short tandem repeat (STR), mutation, single nucleotide alteration, reorganization, substitution or aberrant methylation) that results in a detectable change in the genome or genetic information for a test sample. A genotype and/or genetic variation often is in a particular genomic region (e.g., chromosome, portion of a chromosome (i.e., sub-chromosome region), STR, polymorphic region, translocated region, altered nucleotide sequence, the like or combinations of the foregoing). A genetic variation sometimes is a copy number alteration for a particular region, such as a trisomy or monosomy for chromosome region, or a microduplication or microdeletion event for a particular region (e.g., gain or loss of a region of about 10 megabases or less (e.g., about 9 megabases or less, 8 megabases or less, 7 megabases or less, 6 megabases or less, 5 megabases or less, 4 megabases or less, 3 megabases or less, 2 megabases or less or 1 megabase or less)), for example. A copy number alteration sometimes is expressed as having no copy or one, two, three or four or more copies of a particular region (e.g., chromosome, sub-chromosome, STR, microduplication or microdeletion region).

Presence or absence of genomic instability, a genotype, a phenotype, a genetic variation and/or a medical condition can be determined by transforming, analyzing and/or manipulating sequence reads that have been mapped to genomic portions (e.g., counts, counts of genomic portions of a reference genome). In certain embodiments, an outcome and/or classification is determined according to normalized counts, read densities, read density profiles, and the like, and can be determined by a method described herein. An outcome and/or classification sometimes includes one or more scores and/or calls that refer to the probability that genomic instability, or a particular genotype, phenotype, genetic variation, or medical condition, is present or absent for a test sample. The value of a score may be used to determine, for example, a variation, difference, or ratio of mapped sequence reads that may correspond to genomic instability, a genotype, phenotype, genetic variation, or medical condition. For example, calculating a positive score for genomic instability, or a selected genotype, phenotype, genetic variation, or medical condition, from a data set, with respect to a reference genome, can lead to a classification of genomic instability, or the genotype, phenotype, genetic variation, or medical condition, for a test sample.

Any suitable expression of an outcome and/or classification can be provided. An outcome and/or classification sometimes is based on and/or includes one or more numerical values generated using a processing method described herein in the context of one or more considerations of probability. Non-limiting examples of values that can be utilized include a sensitivity, specificity, standard deviation, median absolute deviation (MAD), measure of certainty, measure of confidence, measure of certainty or confidence that a value obtained for a test sample is inside or outside a particular range of values, measure of uncertainty, measure of uncertainty that a value obtained for a test sample is inside or outside a particular range of values, coefficient of variation (CV), confidence level, confidence interval (e.g., about 95% confidence interval), standard score (e.g., z-score), chi value, phi value, result of a t-test, p-value, ploidy value, fitted minority species fraction, area ratio, median level, the like or combination thereof. In some embodiments, an outcome and/or classification comprises a read density, a read density profile and/or a plot (e.g., a profile plot). In certain embodiments, multiple values are analyzed together, sometimes in a profile for such values (e.g., z-score profile, p-value profile, chi value profile, phi value profile, result of a t-test, value profile, the like, or combination thereof). A consideration of probability can facilitate determining whether a subject is at risk of having, or has, genomic instability, a genotype, phenotype, genetic variation and/or medical condition, and an outcome and/or classification determinative of the foregoing sometimes includes such a consideration.

In certain embodiments, an outcome and/or classification is based on and/or includes a conclusion that predicts and/or determines a risk or probability of the presence or absence of genomic instability, a genotype, phenotype, genetic variation and/or medical condition for a test sample. A conclusion sometimes is based on a value determined from a data analysis method described herein (e.g., a statistics value indicative of probability, certainty and/or uncertainty (e.g., standard deviation, median absolute deviation (MAD), measure of certainty, measure of confidence, measure of certainty or confidence that a value obtained for a test sample is inside or outside a particular range of values, measure of uncertainty, measure of uncertainty that a value obtained for a test sample is inside or outside a particular range of values, coefficient of variation (CV), confidence level, confidence interval (e.g., about 95% confidence interval), standard score (e.g., z-score), chi value, phi value, result of a t-test, p-value, sensitivity, specificity, the like or combination thereof). An outcome and/or classification sometimes is expressed in a laboratory test report (described in greater detail hereafter) for particular test sample as a probability (e.g., odds ratio, p-value), likelihood, or risk factor, associated with the presence or absence of genomic instability, a genotype, phenotype, genetic variation and/or medical condition. An outcome and/or classification for a test sample sometimes is provided as “positive” or “negative” with respect a genomic instability, or a particular genotype, phenotype, genetic variation and/or medical condition. For example, an outcome and/or classification sometimes is designated as “positive” in a laboratory test report for a particular test sample where presence of genomic instability, a genotype, phenotype, genetic variation and/or medical condition is determined, and sometimes an outcome and/or classification is designated as “negative” in a laboratory test report for a particular test sample where absence of genomic instability, a genotype, phenotype, genetic variation and/or medical condition is determined. An outcome and/or classification sometimes is determined and sometimes includes an assumption used in data processing.

An outcome and/or classification sometimes is based on or is expressed as a value in or out of a cluster, value over or under a threshold value, value within a range (e.g., a threshold range), and/or a value with a measure of variance or confidence. In some embodiments, an outcome and/or classification is based on or is expressed as a value above or below a predetermined threshold or cutoff value and/or a measure of uncertainty, confidence level or confidence interval associated with the value. In certain embodiments, a predetermined threshold or cutoff value is an expected level or an expected level range. In some embodiments, a value obtained for a test sample is a standard score (e.g., z-score), where presence of genomic instability, a genotype, phenotype, genetic variation and/or medical condition is determined when the absolute value of the score is greater than a particular score threshold (e.g., threshold between about 2 and about 5; between about 3 and about 4), and where the absence of genomic instability, a genotype, phenotype, genetic variation and/or medical condition is determined when the absolute value of the score is less than the particular score threshold. In certain embodiments, an outcome and/or classification is based on or is expressed as a value that falls within or outside a predetermined range of values (e.g., a threshold range) and the associated uncertainty or confidence level for that value being inside or outside the range. In some embodiments, an outcome and/or classification comprises a value that is equal to a predetermined value (e.g., equal to 1, equal to zero), or is equal to a value within a predetermined value range, and its associated uncertainty or confidence level for that value being equal or within or outside the range. An outcome and/or classification sometimes is graphically represented as a plot (e.g., profile plot). An outcome and/or classification sometimes comprises use of a reference value or reference profile, and sometimes a reference value or reference profile is obtained from one or more reference samples (e.g., reference sample(s) euploid for a selected part of a genome (e.g., region)).

In some embodiments, an outcome and/or classification is based on or includes use of a measure of uncertainty between a test value or profile and a reference value or profile for a selected region. In some embodiments, a determination of the presence or absence of genomic instability, a genotype, phenotype, genetic variation and/or medical condition is according to the number of deviations (e.g., sigma) between a test value or profile and a reference value or profile for a selected region (e.g., a chromosome, or part thereof). A measure of deviation often is an absolute value or absolute measure of deviation (e.g., mean absolute deviation or median absolute deviation (MAD)). In some embodiments, the presence of genomic instability, a genotype, phenotype, genetic variation and/or medical condition is determined when the number of deviations between a test value or profile and a reference value or profile is about 1 or greater (e.g., about 1.5, 2, 2.5, 2.6, 2.7, 2.8, 2.9, 3, 3.1, 3.2, 3.3, 3.4, 3.5, 3.6, 3.7, 3.8, 3.9, 4, 5 or 6 deviations or greater). In certain embodiments, presence of genomic instability, a genotype, phenotype, genetic variation and/or medical condition is determined when a test value or profile and a reference value or profile differ by about 2 to about 5 measures of deviation (e.g., sigma, MAD), or more than 3 measures of deviation (e.g., 3 sigma, 3 MAD). A deviation of greater than three between a test value or profile and a reference value or profile often is indicative of a non-euploid test subject (e.g., presence of a genetic variation (e.g., presence of trisomy, monosomy, microduplication, microdeletion) for a selected region. Test values or profiles significantly above a reference profile, which reference profile is indicative of euploidy, sometimes are determinative of a trisomy, sub-chromosome duplication or microduplication. Test values or profiles significantly below a reference profile, which reference profile is indicative of euploidy, sometimes are determinative of a monosomy, sub-chromosome deletion or microdeletion. In some embodiments, the absence of genomic instability, a genotype, phenotype, genetic variation and/or medical condition is determined when the number of deviations between a test value or profile and reference value or profile for a selected region of a genome is about 3.5 or less (e.g., about less than about 3.4, 3.3, 3.2, 3.1, 3, 2.9, 2.8, 2.7, 2.6, 2.5, 2.4, 2.3, 2.2, 2.1, 2, 1.9, 1.8, 1.7, 1.6, 1.5, 1.4, 1.3, 1.2, 1.1, 1 or less). In certain embodiments, absence of genomic instability, a genotype, phenotype, genetic variation and/or medical condition is determined when a test value or profile differs from a reference value or profile by less than three measures of deviation (e.g., 3 sigma, 3 MAD). In some embodiments, a measure of deviation of less than three between a test value or profile and reference value or profile (e.g., 3-sigma for standard deviation) often is indicative of a region that is euploid (e.g., absence of a genetic variation). A measure of deviation between a test value or profile for a test sample and a reference value or profile for one or more reference subjects can be plotted and visualized (e.g., z-score plot).

In some embodiments, an outcome and/or classification is determined according to a call zone. In certain embodiments, a call is made (e.g., a call determining presence or absence of genomic instability, a genotype, phenotype, genetic variation and/or medical condition) when a value (e.g., a profile, a read density profile and/or a measure of uncertainty) or collection of values falls within a pre-defined range (e.g., a zone, a call zone). In some embodiments, a call zone is defined according to a collection of values (e.g., profiles, read density profiles, measures or determination of probability and/or measures of uncertainty) obtained from a particular group of samples. In certain embodiments, a call zone is defined according to a collection of values that are derived from the same chromosome or part thereof. In some embodiments, a call zone for determining presence or absence of genomic instability, a genotype, phenotype, genetic variation and/or medical condition is defined according a measure of uncertainty (e.g., high level of confidence or low measure of uncertainty) and/or a quantification of a minority nucleic acid species (e.g., about 1% minority species or greater (e.g., about 2, 3, 4, 5, 6, 7, 8, 9, 10% or more minority nucleic acid species)) determined for a test sample. A minority nucleic acid species quantification sometimes is a fraction or percent of cancer cell nucleic acid or fetal nucleic acid (i.e., fetal fraction) ascertained for a test sample. In some embodiments, a call zone is defined by a confidence level or confidence interval (e.g., a confidence interval for 95% level of confidence). A call zone sometimes is defined by a confidence level, or confidence interval based on a particular confidence level, of about 90% or greater (e.g., about 91, 92, 93, 94, 95, 96, 97, 98, 99, 99.1, 99.2, 99.3, 99.4, 99.5, 99.6, 99.7, 99.8, 99.9% or greater). In some embodiments, a call is made using a call zone and additional data or information. In some embodiments, a call is made without using a call zone. In some embodiments, a call is made based on a comparison without the use of a call zone. In some embodiments, a call is made based on visual inspection of a profile (e.g., visual inspection of read densities).

In some embodiments, a classification or call is not provided for a test sample when a test value or profile is in a no-call zone. In some embodiments, a no-call zone is defined by a value (e.g., collection of values) or profile that indicates low accuracy, high risk, high error, low level of confidence, high measure of uncertainty, the like or combination thereof. In some embodiments, a no-call zone is defined, in part, by a minority nucleic acid species quantification (e.g., a minority nucleic acid species of about 10% or less (e.g., about 9%, 8%, 7%, 6%, 5%, 4%, 3%, 2%, 1.5%, 1% or less minority nucleic acid species)). An outcome and/or classification generated for determining presence or absence of genomic instability, a genotype, phenotype, genetic variation and/or medical condition sometimes includes a null result. A null result sometimes is a data point between two clusters; sometimes is a numerical value with a standard deviation that encompasses values for both the presence and absence of genomic instability, a genotype, phenotype, genetic variation and/or medical condition; and sometimes is a data set with a profile plot that is not similar to profile plots for subjects having or free from genomic instability, or a genotype, phenotype, genetic variation or medical condition under investigation. In some embodiments, an outcome and/or classification indicative of a null result is considered a determinative result, and the determination can include a conclusion of the need for additional information and/or a repeat of data generation and/or analysis for determining the presence or absence of genomic instability, a genotype, phenotype, genetic variation and/or medical condition.

In some instances, four types of classifications generated in a classification process: true positive, false positive, true negative and false negative. The term “true positive” as used herein refers to presence of genomic instability, a genotype, phenotype, genetic variation, or medical condition correctly determined for a test sample. The term “false positive” as used herein refers to presence of genomic instability, a genotype, phenotype, genetic variation, or medical condition incorrectly determined for a test sample. The term “true negative” as used herein refers to absence of genomic instability, a genotype, phenotype, genetic variation, or medical condition correctly determined for a test sample. The term “false negative” as used herein refers to absence of genomic instability, a genotype, phenotype, genetic variation, or medical condition incorrectly determined for a test sample. Two measures of performance for a classification process can be calculated based on the ratios of these occurrences: (i) a sensitivity value, which generally is the fraction of predicted positives that are correctly identified as being positives; and (ii) a specificity value, which generally is the fraction of predicted negatives correctly identified as being negative.

At block 135, the results of the analysis may be filtered using a filter subsystem. In some instances, the results of the analysis are filtered by strength and/or significance. In certain instances, the results of the analysis are filtered by strength and/or significance of the identified genomic instability. The strength and/or significance may be determined using one or more processes as described herein. For example, a filtering step can comprise the use of one or more statistical algorithms, decision analysis, comparisons, and/or the like processing steps, as described in detail herein.

In some embodiments, the results of the analysis can be filtered using any suitable number of statistical algorithms. Non-limiting examples of statistical algorithms suitable for use with filtering described herein include principal component analysis, decision trees, counternulls, multiple comparisons, omnibus test, Behrens-Fisher problem, bootstrapping, Fisher's method for combining independent tests of significance, null hypothesis, type I error, type II error, exact test, one-sample Z test, two-sample Z test, one-sample t-test, paired t-test, two-sample pooled t-test having equal variances, two-sample unpooled t-test having unequal variances, one-proportion z-test, two-proportion z-test pooled, two-proportion z-test unpooled, one-sample chi-square test, two-sample F test for equality of variances, confidence interval, credible interval, significance, meta analysis, simple linear regression, robust linear regression, the like or combinations of the foregoing. I

In some embodiments, the results of the analysis can be filtered by preforming a comparison (e.g., comparing a test profile to a reference profile). Two or more data sets, two or more relationships and/or two or more profiles can be compared by a suitable method. Non-limiting examples of statistical methods suitable for comparing data sets, relationships and/or profiles include Behrens-Fisher approach, bootstrapping, Fisher's method for combining independent tests of significance, Neyman-Pearson testing, confirmatory data analysis, exploratory data analysis, exact test, F-test, Z-test, T-test, calculating and/or comparing a measure of uncertainty, a null hypothesis, counternulls and the like, a chi-square test, omnibus test, calculating and/or comparing level of significance (e.g., statistical significance), a meta analysis, a multivariate analysis, a regression, simple linear regression, robust linear regression, the like or combinations of the foregoing. In certain embodiments comparing two or more data sets, relationships and/or profiles comprises determining and/or comparing a measure of uncertainty.

### 4) Reporting/Output

At block 140, a laboratory test report is generated for the results of the analysis and/or the filtered results of the analysis using a report subsystems. In some instances, the filtering and/or laboratory test report process includes a measure of test performance (e.g., sensitivity and/or specificity) and/or a measure of confidence (e.g., a confidence level, confidence interval). A measure of test performance and/or confidence sometimes may be obtained from a clinical validation study performed prior to performing a laboratory test for a test sample. In certain embodiments, one or more of sensitivity, specificity and/or confidence are expressed as a percentage. In some embodiments, a percentage expressed independently for each of sensitivity, specificity or confidence level, is greater than about 90% (e.g., about 90, 91, 92, 93, 94, 95, 96, 97, 98 or 99%, or greater than 99% (e.g., about 99.5%, or greater, about 99.9% or greater, about 99.95% or greater, about 99.99% or greater)). A confidence interval expressed for a particular confidence level (e.g., a confidence level of about 90% to about 99.9% (e.g., about 95%)) can be expressed as a range of values, and sometimes is expressed as a range or sensitivities and/or specificities for a particular confidence level. Coefficient of variation (CV) in some embodiments is expressed as a percentage, and sometimes the percentage is about 10% or less (e.g., about 10, 9, 8, 7, 6, 5, 4, 3, 2 or 1%, or less than 1% (e.g., about 0.5% or less, about 0.1% or less, about 0.05% or less, about 0.01% or less)). A probability (e.g., that a particular outcome and/or classification is not due to chance) in certain embodiments is expressed as a standard score (e.g., z-score), a p-value, or result of a t-test. In some embodiments, a measured variance, confidence level, confidence interval, sensitivity, specificity and the like (e.g., referred to collectively as confidence parameters) for an outcome and/or classification can be generated using one or more data processing manipulations described herein. Specific examples of generating an outcome and/or classification and associated confidence levels are described, for example, in International Patent Application Publication Nos. WO 2013/052913, WO 2014/190286 and WO 2015/051163, the entire content of which is incorporated herein by reference for all purposes.

An outcome and/or classification for a test sample often is ordered by, and often is provided to, a health care professional or other qualified individual (e.g., physician or assistant) who transmits an outcome and/or classification to a subject from whom the test sample is obtained. In certain embodiments, an outcome and/or classification is provided using a suitable visual medium (e.g., a peripheral or component of a machine, e.g., a printer or display). A classification and/or outcome often is provided to a healthcare professional or qualified individual in the form of a report. A report typically comprises a display of an outcome and/or classification (e.g., a value, or an assessment or probability of presence or absence of genomic instability, a genotype, phenotype, genetic variation and/or medical condition), sometimes includes an associated confidence parameter, and sometimes includes a measure of performance for a test used to generate the outcome and/or classification. A report sometimes includes a recommendation for a follow-up procedure (e.g., a procedure that confirms the outcome or classification). A report sometimes includes a visual representation of a chromosome or portion thereof (e.g., a chromosome ideogram or karyogram), and sometimes shows a visualization of a duplication and/or deletion region for a chromosome (e.g., a visualization of a whole chromosome for a chromosome deletion or duplication; a visualization of a whole chromosome with a deleted region or duplicated region shown; a visualization of a portion of chromosome duplicated or deleted; a visualization of a portion of a chromosome remaining in the event of a deletion of a portion of a chromosome) identified for a test sample.

A report can be displayed in a suitable format that facilitates determination of presence or absence of genomic instability, a genotype, a phenotype, a genetic variation and/or a medical condition by a health professional or other qualified individual. Non-limiting examples of formats suitable for use for generating a report include digital data, a graph, a 2D graph, a 3D graph, and 4D graph, a picture (e.g., a jpg, bitmap (e.g., bmp), pdf, tiff, gif, raw, png, the like or suitable format), a pictograph, a chart, a table, a bar graph, a pie graph, a diagram, a flow chart, a scatter plot, a map, a histogram, a density chart, a function graph, a circuit diagram, a block diagram, a bubble map, a constellation diagram, a contour diagram, a cartogram, spider chart, Venn diagram, nomogram, and the like, or combination of the foregoing.

A report may be generated by a computer and/or by human data entry, and can be transmitted and communicated using a suitable electronic medium (e.g., via the internet, via computer, via facsimile, from one network location to another location at the same or different physical sites), or by another method of sending or receiving data (e.g., mail service, courier service and the like). Non-limiting examples of communication media for transmitting a report include auditory file, computer readable file (e.g., pdf file), paper file, laboratory file, medical record file, or any other medium described in the previous paragraph. A laboratory file or medical record file may be in tangible form or electronic form (e.g., computer readable form), in certain embodiments. After a report is generated and transmitted, a report can be received by obtaining, via a suitable communication medium, a written and/or graphical representation comprising an outcome and/or classification, which upon review allows a healthcare professional or other qualified individual to make a determination as to presence or absence of genomic instability, a genotype, phenotype, genetic variation and/or or medical condition for a test sample.

An outcome and/or classification may be provided by and obtained from a laboratory (e.g., obtained from a laboratory file). A laboratory file can be generated by a laboratory that carries out one or more tests for determining presence or absence of genomic instability, a genotype, a phenotype, a genetic variation and/or a medical condition for a test sample. Laboratory personnel (e.g., a laboratory manager) can analyze information associated with test samples (e.g., test profiles, reference profiles, test values, reference values, level of deviation, patient information) underlying an outcome and/or classification. For calls pertaining to presence or absence of genomic instability, a genotype, a phenotype, a genetic variation and/or a medical condition that are close or questionable, laboratory personnel can re-run the same procedure using the same (e.g., aliquot of the same sample) or different test sample from a test subject. A laboratory may be in the same location or different location (e.g., in another country) as personnel assessing presence or absence of genomic instability, phenotype, genetic variation and/or medical condition from the laboratory file. For example, a laboratory file can be generated in one location and transmitted to another location in which the information for a test sample therein is assessed by a healthcare professional or other qualified individual, and optionally, transmitted to the subject from which the test sample was obtained. A laboratory sometimes generates and/or transmits a laboratory report containing a classification of presence or absence of genomic instability, a genotype, phenotype, a genetic variation and/or a medical condition for a test sample. A laboratory generating a laboratory test report sometimes is a certified laboratory, and sometimes is a laboratory certified under the Clinical Laboratory Improvement Amendments (CLIA).

An outcome and/or classification sometimes is a component of a diagnosis for a subject, and sometimes an outcome and/or classification is utilized and/or assessed as part of providing a diagnosis for a test sample. For example, a healthcare professional or other qualified individual may analyze an outcome and/or classification and provide a diagnosis based on, or based in part on, the outcome and/or classification. In some embodiments, determination, detection or diagnosis of a medical condition, disease, syndrome or abnormality comprises use of an outcome and/or classification determinative of presence or absence of genomic instability, a genotype, a phenotype, a genetic variation and/or a medical condition. In some embodiments, an outcome and/or classification based on counted mapped sequence reads, normalized counts and/or transformations thereof is determinative of presence or absence of genomic instability, a genotype and/or a genetic variation. In certain embodiments, a diagnosis comprises a determination of presence or absence of a condition, syndrome or abnormality. In certain instances, a diagnosis comprises a determination of a genetic variation as the nature and/or cause of genomic instability and/or a medical condition, disease, syndrome or abnormality. Thus, provided herein are methods for diagnosing presence or absence of genomic instability, a genotype, phenotype, a genetic variation and/or a medical condition for a test sample according to an outcome or classification generated by methods described herein, and optionally according to generating and transmitting a laboratory report that includes a classification for presence or absence of genomic instability, a genotype, phenotype, a genetic variation and/or a medical condition for the test sample.

An outcome and/or classification sometimes is a component of health care and/or treatment of a subject. An outcome and/or classification sometimes is utilized and/or assessed as part of providing a treatment for a subject from whom a test sample was obtained. For example, an outcome and/or classification indicative of presence or absence of genomic instability, a genotype, a phenotype, a genetic variation, and/or a medical condition is a component of health care and/or treatment of a subject from whom a test sample was obtained. Medical care, treatment and or diagnosis can be in any suitable area of health, such as medical treatment of subjects for prenatal care, cell proliferative conditions, cancer and the like, for example. An outcome and/or classification determinative of presence or absence of genomic instability, a genotype, a phenotype, a genetic variation, and/or a medical condition, disease, syndrome or abnormality by methods described herein sometimes is independently verified by further testing. Any suitable type of further test to verify an outcome and/or classification can be utilized, non-limiting examples of which include blood level test (e.g., serum test), biopsy, scan (e.g., CT scan, MRI scan), invasive sampling (e.g., amniocentesis or chorionic villus sampling), karyotyping, microarray assay, ultrasound, sonogram, and the like, for example.

A healthcare professional or qualified individual can provide a suitable healthcare recommendation based on the outcome and/or classification provided in a laboratory report. In some embodiments, a recommendation is dependent on the outcome and/or classification provided (e.g., cancer, stage and/or type of cancer, Down's syndrome, Turner syndrome, medical conditions associated with genetic variations in T13, medical conditions associated with genetic variations in T18). Non-limiting examples of recommendations that can be provided based on an outcome or classification in a laboratory report includes, without limitation, surgery, radiation therapy, chemotherapy, genetic counseling, after-birth treatment solutions (e.g., life planning, long term assisted care, medicaments, symptomatic treatments), pregnancy termination, organ transplant, blood transfusion, further testing described in the previous paragraph, the like or combinations of the foregoing. Thus, methods for treating a subject and methods for providing health care to a subject sometimes include generating a classification for presence or absence of genomic instability, a genotype, phenotype, a genetic variation and/or a medical condition for a test sample by a method described herein, and optionally generating and transmitting a laboratory report that includes a classification of presence or absence of genomic instability, a genotype, phenotype, a genetic variation and/or a medical condition for the test sample.

Generating an outcome and/or classification can be viewed as a transformation of nucleic acid sequence reads from a test sample into a representation of a subject's cellular nucleic acid. For example, transmuting sequence reads of nucleic acid from a subject by a method described herein, and generating an outcome and/or classification can be viewed as a transformation of relatively small sequence read fragments to a representation of relatively large and complex structure of nucleic acid in the subject. In some embodiments, an outcome and/or classification results from a transformation of sequence reads from a subject into a representation of an existing nucleic acid structure present in the subject (e.g., a genome, a chromosome, chromosome segment, mixture of circulating cell-free nucleic acid fragments in the subject).

In some embodiments, a method herein comprises treating a subject when the presence of a genetic alteration or genetic variation is determined for a test sample from the subject. In some embodiments, treating a subject comprises performing a medical procedure when the presence of a genetic alteration or genetic variation is determined for a test sample. In some embodiments, a medical procedure includes an invasive diagnostic procedure such as, for example, amniocentesis, chorionic villus sampling, biopsy, and the like. For example, a medical procedure comprising amniocentesis or chorionic villus sampling may be performed when the presence of a fetal aneuploidy is determined for a test sample from a pregnant female. In another example, a medical procedure comprising a biopsy may be performed when presence of a genetic alteration indicative of or associated with the presence of cancer is determined for a test sample from a subject. An invasive diagnostic procedure may be performed to confirm a determination of the presence of a genetic alteration or genetic variation and/or may be performed to further characterize a medical condition associated with a genetic alteration or genetic variation, for example. In some embodiments, a medical procedure may be performed as a treatment of a medical condition associated with a genetic alteration or genetic variation. Treatments may include one or more of surgery, radiation therapy, chemotherapy, pregnancy termination, organ transplant, cell transplant, blood transfusion, medicaments, symptomatic treatments, and the like, for example.

In some embodiments, a method herein comprises treating a subject when the absence of a genetic alteration or genetic variation is determined for a test sample from the subject. In some embodiments, treating a subject comprises performing a medical procedure when the absence of a genetic alteration or genetic variation is determined for a test sample. For example, when the absence of a genetic alteration or genetic variation is determined for a test sample, a medical procedure may include health monitoring, retesting, further screening, follow-up examinations, and the like. In some embodiments, a method herein comprises treating a subject consistent with a euploid pregnancy or normal pregnancy when the absence of a fetal aneuploidy, genetic variation or genetic alteration is determined for a test sample from a pregnant female. For example, a medical procedure consistent with a euploid pregnancy or normal pregnancy may be performed when the absence of a fetal aneuploidy, genetic variation or genetic alteration is determined for a test sample from a pregnant female. A medical procedure consistent with a euploid pregnancy or normal pregnancy may include one or more procedures performed as part of monitoring health of the fetus and/or the mother, or monitoring feto-maternal well-being. A medical procedure consistent with a euploid pregnancy or normal pregnancy may include one or more procedures for treating symptoms of pregnancy which may include, for example, one or more of nausea, fatigue, breast tenderness, frequent urination, back pain, abdominal pain, leg cramps, constipation, heartburn, shortness of breath, hemorrhoids, urinary incontinence, varicose veins and sleeping problems. A medical procedure consistent with a euploid pregnancy or normal pregnancy may include one or more procedures performed throughout the course of prenatal care for assessing potential risks, treating complications, addressing preexisting medical conditions (e.g., hypertension, diabetes), and monitoring the growth and development of the fetus, for example. Medical procedures consistent with a euploid pregnancy or normal pregnancy may include, for example, complete blood count (CBC) monitoring, Rh antibody testing, urinalysis, urine culture monitoring, rubella screening, hepatitis B and hepatitis C screening, sexually transmitted infection (STI) screening (e.g., screening for syphilis, chlamydia, gonorrhea), human immunodeficiency virus (HIV) screening, tuberculosis (TB) screening, alpha-fetoprotein screening, fetal heart rate monitoring (e.g., using an ultrasound transducer), uterine activity monitoring (e.g., using toco transducer), genetic screening and/or diagnostic testing for genetic disorders (e.g., cystic fibrosis, sickle cell anemia, hemophilia A), glucose screening, glucose tolerance testing, treatment of gestational diabetes, treatment of prenatal hypertension, treatment of preeclampsia, group B streptococci (GBS) blood type screening, group B strep culture, treatment of group B strep (e.g., with antibiotics), ultrasound monitoring (e.g., routine ultrasound monitoring, level II ultrasound monitoring, targeted ultrasound monitoring), non-stress test monitoring, biophysical profile monitoring, amniotic fluid index monitoring, serum testing (e.g., plasma protein-A (PAPP-A), alpha-fetoprotein (AFP), human chorionic gonadotropin (hCG), unconjugated estriol (uE3), and inhibin-A (inhA) testing), genetic testing, amniocentesis diagnostic testing and chorionic villus sampling (CVS) diagnostic testing.

In some embodiments, a method herein comprises treating a subject consistent with having no cancer when the absence of a genetic variation or genetic alteration is determined for a test sample from a subject. In certain embodiments, a medical procedure consistent with a healthy prognosis may be performed when absence of a genetic alteration or genetic variation associated with cancer is determined for a test sample. For example, medical procedures consistent with a healthy prognosis include without limitation monitoring health of the subject from whom a test sample was tested, performing a secondary test (e.g., a secondary screening test), performing a confirmatory test, monitoring one or more biomarkers associated with cancer (e.g., prostate specific antigen (PSA) in males), monitoring blood cells (e.g., red blood cells, white blood cells, platelets), monitoring one or more vital signs (e.g., heart rate, blood pressure), and/or monitoring one or more blood metabolites (e.g., total cholesterol, HDL (high-density lipoprotein), LDL (low-density lipoprotein), triglycerides, total cholesterol/HDL ratio, glucose, fibrinogen, hemoglobin, dehydroepiandrosterone (DHEA), homocysteine, C-reactive protein, hormones (e.g., thyroid stimulating hormone, testosterone, estrogen, estradiol), creatine, salt (e.g., potassium, calcium), and the like). In some embodiments, a method herein comprises performing no medical procedure, and sometimes no medical procedure that includes invasive sampling, when the absence of a genetic alteration or genetic variation is determined for a test sample.

### D. POSITIVE CFDNA SCREENING PIPELINE

The techniques described herein include clinically reliable methods and systems for detecting a genetic variation using circulating cell-free nucleic acid in a sample from a subject, classifying an origin of the genetic variation, and recommending a follow-up screening for the subject. In some instances, the subject may have conducted a screening test and got a positive or suspected genetic variation result before conducting the cfDNA screening analysis. In such instances, the disclosed techniques provide advantages in confirming or disconfirming the prior test results, and further provide detailed information such as an origin of the genetic variation and/or a size/location of the genetic variation. A prior screening may also help focus the cfDNA screening analysis described herein on the affected region of the genome to provide a cost and computing efficient cfDNA screening analysis. In other instances, the techniques described herein detect genetic variations in a sample without prior positive/suspected results.

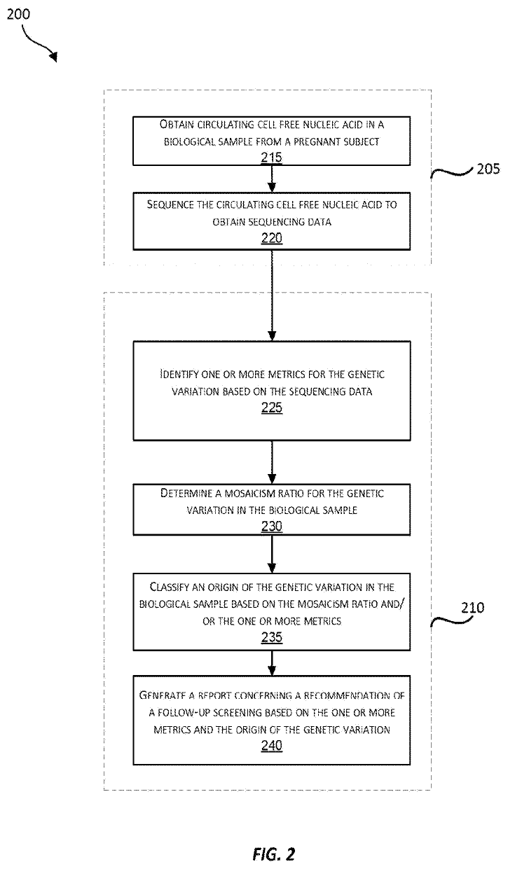

FIG. 2 is a flowchart illustrating a process 200 for classifying an origin of a genetic variation in a biological sample in accordance with various embodiments. The processing depicted in FIG. 2 may be implemented in software (e.g., code, instructions, program) executed by one or more processing units (e.g., processors, cores) of the respective systems, hardware, or combinations thereof (e.g., the intelligent selection machine). The software may be stored on a non-transitory store medium (e.g., on a memory device). The method presented in FIG. 2 and described below is intended to be illustrative and non-limiting. Although FIG. 2 depicts the various processing steps occurring in a particular sequence or order, this is not intended to be limiting. In certain alternative embodiments, the steps may be performed in some different orders, or some steps may also be performed in parallel. In certain embodiments, the processing or a portion of the processing depicted in FIG. 2 may be performed by the clinical test pipeline 100 as described with respect to FIG. 1.

The process 200 can be executed in two stages. At a first stage 205, sequencing data is obtained (as described with respect to FIG. 1). In some embodiments, the sequencing data comprise sequencing reads and/or mapping or aligning information of the sequence reads (e.g., alignment scores, start and end positions of sequence reads, and the like). Stage 205 comprises two blocks 215 and 220.

At block 215, circulating cell free nucleic acid is obtained in a biological sample from a pregnant subject. The circulating cell free nucleic acid may comprise maternal nucleic acid and fetal nucleic acid. The circulating cell free nucleic acid may be obtained or captured by probe oligonucleotides under hybridization conditions. In some embodiments, the biological sample may be a maternal blood sample. The biological sample of the pregnant subject may have been determined to have the genetic variation, and the determination may be made by assay screening, such as FISH, microarray, karyotype, or the like. The circulating cell free nucleic acid can be cell-free DNA (cfDNA), circulating tumor DNA (ctDNA), and/or cell-free RNA (cfRNA or exRNA).

The obtained circulating cell free nucleic acid may be enriched. In some embodiments, the circulating cell free nucleic acid enrichment is performed using a selective enrichment method. This is because higher fetal fraction may increase the sensitivity of NIPT, which leads to a higher likelihood and accuracy of detecting the genetic variation and identifying the origin of the genetic variation. In some embodiments, the fetal nucleic acid is enriched. Fetal nucleic acid enrichment methods include but not limited to: i) size-based separation approaches: ii) magnetic bead-based approaches: methylation-based approaches; iv) PCR-based approaches; v) other physical or biochemical methods. For example, single nucleotide polymorphisms (SNPs) differ between nucleic acid from the mother and the fetus, and they can be used to distinguish and selectively amplify fetal nucleic acid. Techniques like digital PCR can be employed for SNP-based enrichment. In some embodiments, a biochemical enrichment technique applies specific enzymes that targets and digest longer (maternal) nucleic acid molecules, leaving the shorter fetal nucleic acid intact. In some embodiments, another biochemical enrichment technique targets a specific biochemical character of the fetal nucleic acid, and binds the fetal nucleic acid based on the specific biochemical character. The enrichment of fetal nucleic acid can be achieved by utilizing multiple enrichment methods.

At block 220, the circulating cell free nucleic acid is sequenced using sequencing technology such as a massively parallel sequencing (MPS) platform to obtain sequencing data. MPS allows for the simultaneous sequencing of millions of DNA fragments, enabling the rapid and cost-effective generation of large amounts of sequencing data. In some embodiments, the sequencing is whole-genome sequencing. In some embodiments, the sequencing is whole-exome sequencing. In some embodiments, the sequencing is performed to the circulating cell free nucleic acid of a genomic region of interest. Targeted sequencing may also be performed to enrich a certain type of genetic variations (e.g., SNPs that are specific to the fetal nucleic acid). The genomic region of interest may be determined by the genetic variation. In some embodiments, at least thousands to millions of sequence reads are obtained by the sequencing. In some embodiments, a single sequencing run generates about 100 million to 1 billion sequence reads.

At a second stage 210, information regarding the genetic variation is identified or determined based on the sequencing data, and a report can be generated based on the information regarding the genetic variation. In some embodiments, the information regarding the genetic variation comprises an origin of the genetic variation, a probability or likelihood of the origin of the genetic variation, a size of the genetic variation, and/or a location of the genetic variation. Stage 210 comprises four blocks 225, 230, 235, and 240.

At block 225, one or more metrics for the genetic variation is identified based on the sequencing data. The one or more metrics may comprise the size of the genetic variation and/or the location of the genetic variation. In some embodiments, the location of the genetic variation is a breakpoint of the genetic variation. In some embodiments, the one or more metrics for the genetic variation may be identified by comparing the sequencing data with data of a reference genome or data of a region of the reference genome. In some embodiments, the reference genome is at least hundreds of thousands base pairs long. The length of the reference genome may range from 1 million to several billion base pairs. In some embodiments, the reference genome is at least 46 million base pair long. In some embodiments, the region of the reference genome is at least one thousand, ten thousand, a hundred thousand, one million, two million, or three million base pairs long.

At block 230, a mosaicism ratio (MR) for the genetic variation in the biological sample is determined. The determination of the MR may be based on the sequencing data and/or the one or more metrics. In various embodiments, the MR is calculated by: (a) determining a fraction of nucleic acid having the genetic variation in the circulating cell free nucleic acid based on the sequencing data and/or the one or more metrics, (b) determining a fraction of the fetal nucleic acid in the circulating cell free nucleic acid based on the sequencing data and/or the one or more metrics, and (c) generating the MR using the fraction of (a) to the fraction of (b).

At block 235, the origin of the genetic variation in the biological sample is classified based on the mosaicism ratio and/or the one or more metrics. In some embodiments, the origin of the genetic variation is classified as maternal when the mosaicism ratio is greater than or equal to a threshold. The threshold may be predetermined using a machine-learning training process, and/or may be adjusted by an authorized practitioner, such as a laboratory director. The threshold may be an empirical threshold based on a statistical analysis. The threshold may depend on the genetic variation. For example, when the genetic variation is a 22q11.2DS, the threshold may be determined to be 3. That is, when the mosaicism ratio for 22q11.2DS of the biological sample is greater than or equal to 3, the origin of the genetic variation is classified to be maternal. In some embodiments, the origin of the genetic variation is classified as fetal when the mosaicism ratio is less than a threshold.

A same threshold may be used for maternal and fetal classification. In some embodiments, the threshold for fetal classification is 3, and when the MR of the biological sample is less than 3, the origin of the genetic variation is classified to be fetal. The threshold for fetal classification may be different from the threshold for maternal. In some embodiments, the threshold for fetal classification is exactly or about 2, 1.9, 1.8, 1.7, 1.6, 1.5, 1.4, 1.3, 1.2, 1.1, or 1. In some embodiments, the classification of the origin or the threshold is independent of the number of fetuses the pregnant woman is carrying at the time the biological sample is obtained. In some embodiments, the threshold for fetal classification or for maternal classification is adjusted based on the number of fetuses the pregnant woman is carrying at the time the biological sample is obtained. The adjustment may be made using a pretrained machine-learning model, or by an authorized practitioner.

In some embodiment, a probability or likelihood of the origin of the genetic variation is predicted at block 235, e.g., 80% that the genetic variation is originated from the maternal nucleic acid. In some embodiment, the probability or likelihood is predicted using a pre-trained machine-learning model based on the MR and/or a predetermined threshold. In some embodiment, the probability or likelihood is affected by the number of fetuses the pregnant woman is carrying at the time the biological sample is obtained, or by the number of fetuses that are suspect to carry the genetic variation. The probability or the likelihood may be further used for downstream applications such as determining whether or not administering a follow-up screening test.

At block 240, a report is generated concerning a recommendation of a follow-up screening based on the one or more metrics and the origin of the genetic variation. The report may further comprise the one or more metrics for the genetic variation, the MR for the genetic variation, the origin of the genetic variation and/or its associated probability or likelihood, partial or all of the sequencing data, available details regarding the pregnant subject, and/or statistics thereof. In some embodiments, the recommendation is not to perform a specific screening, such as an invasive testing, or a FISH screening. In some embodiments, the recommendation is to perform a specific screening or analysis, such as an invasive testing, or a microarray analysis. The report may further provide a recommendation regarding a specific genomic region or a target sequence that the specific screening is focused on. The specific genomic region or the target sequence is the location/sequence whether the genetic variation occurs. The report may be generated such that it links the genetic variation information to the follow-up recommendations in an intuitive and comprehensible graphical user interface. In some instances, the graphical user interface renders the genetic variation information and the follow-up recommendations with additional patient information/data such as results from other assays or tests performed on the biological sample or a different biological sample.

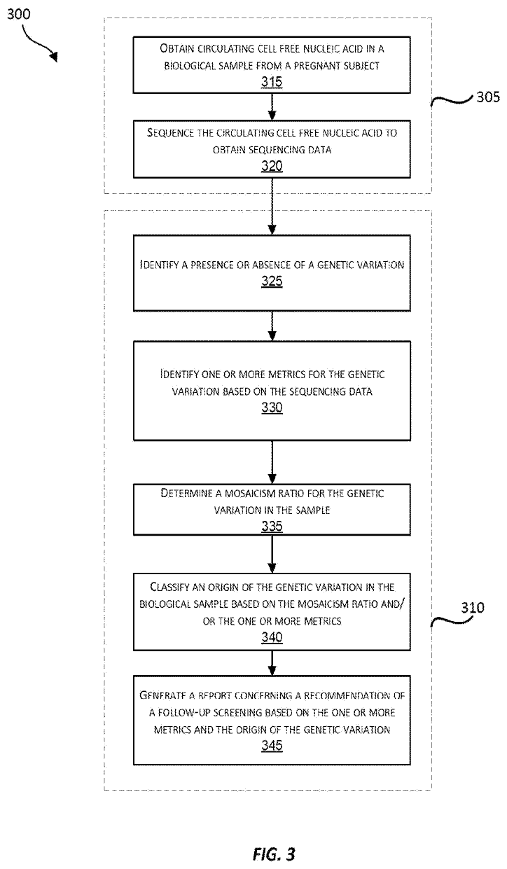

FIG. 3 is a flowchart illustrating a process 300 for identifying a genetic variation and classifying an origin of the genetic variation in accordance with various embodiments. The processing depicted in FIG. 3 may be implemented in software (e.g., code, instructions, program) executed by one or more processing units (e.g., processors, cores) of the respective systems, hardware, or combinations thereof (e.g., the intelligent selection machine). The software may be stored on a non-transitory store medium (e.g., on a memory device). The method presented in FIG. 3 and described below is intended to be illustrative and non-limiting. Although FIG. 3 depicts the various processing steps occurring in a particular sequence or order, this is not intended to be limiting. In certain alternative embodiments, the steps may be performed in some different orders, or some steps may also be performed in parallel. In certain embodiments, the processing or a portion of the processing depicted in FIG. 3 may be performed by the clinical test pipeline 100 as described with respect to FIG. 1.

The process 300 can be executed in two stages. At a first stage 305, sequencing data is obtained (as described with respect to FIG. 1). In some embodiments, the sequencing data comprise sequencing reads and/or mapping or aligning information of the sequence reads (e.g., alignment scores, start and end positions of sequence reads, and the like). Stage 305 comprises two blocks 315 and 320.

At block 315, circulating cell free nucleic acid is obtained in a biological sample from a pregnant subject. The circulating cell free nucleic acid may comprise maternal nucleic acid and fetal nucleic acid. The circulating cell free nucleic acid may be obtained or captured by probe oligonucleotides under hybridization conditions. In some embodiments, the biological sample may be a maternal blood sample. The circulating cell free nucleic acid can be cell-free DNA (cfDNA), circulating tumor DNA (ctDNA), and/or cell-free RNA (cfRNA or exRNA).

The obtained circulating cell free nucleic acid may be enriched. In some embodiments, the circulating cell free nucleic acid enrichment is performed using a selective enrichment method. This is because higher fetal fraction may increase the sensitivity of NIPT, which leads to a higher likelihood and accuracy of detecting the genetic variation and identifying the origin of the genetic variation. In some embodiments, the fetal nucleic acid is enriched. Fetal nucleic acid enrichment methods include but not limited to: i) size-based separation approaches; ii) magnetic bead-based approaches; methylation-based approaches; iv) PCR-based approaches; v) other physical or biochemical methods. For example, single nucleotide polymorphisms (SNPs) differ between nucleic acid from the mother and the fetus, and they can be used to distinguish and selectively amplify fetal nucleic acid. Techniques like digital PCR can be employed for SNP-based enrichment. In some embodiments, a biochemical enrichment technique applies specific enzymes that targets and digest longer (maternal) nucleic acid molecules, leaving the shorter fetal nucleic acid intact. In some embodiments, another biochemical enrichment technique targets a specific biochemical character of the fetal nucleic acid, and binds the fetal nucleic acid based on the specific biochemical character. The enrichment of fetal nucleic acid can be achieved by utilizing multiple enrichment methods.

At block 320, the circulating cell free nucleic acid is sequenced using sequencing technology such as a massively parallel sequencing (MPS) platform to obtain sequencing data. In some embodiments, the sequencing is whole-genome sequencing. In some embodiments, the sequencing is whole-exome sequencing. In some embodiments, the sequencing is performed to the circulating cell free nucleic acid of a genomic region of interest. The genomic region of interest may be determined by a genetic variation to be identified. Targeted sequencing may also be performed to enrich a certain type of genetic variations (e.g., SNPs that are specific to the fetal nucleic acid). In some embodiments, at least thousands to millions of sequence reads are obtained by the sequencing. In some embodiments, a single sequencing run generates about 100 million to 1 billion sequence reads.

At a second stage 310, information regarding a genetic variation is identified or determined based on the sequencing data, and a report can be generated based on the information regarding the genetic variation. In some embodiments, the information regarding the genetic variation comprises a presence or absence of the genetic variation, an origin of the genetic variation, a probability or likelihood of the origin of the genetic variation, a size of the genetic variation, and/or a location of the genetic variation. The stage 310 comprises five blocks 325, 330, 335, 340, and 345

At block 325, a presence or absence of the genetic variation is determined or identified. The identification/determination may be based on a screening test (e.g., an ultrasound screening) or based on the sequencing data. In some embodiments, the presence or absence of the genetic variation may be identified by comparing the sequencing data to data of a reference genome, or a region of the reference genome. In some embodiments, the reference genome is at least hundreds of thousands base pairs long. The length of the reference genome may range from 1 million to several billion base pairs. In some embodiments, the reference genome is at least 46 million base pair long. In some embodiments, the region of the reference genome is at least one thousand, ten thousand, a hundred thousand, one million, two million, or three million base pairs long.

At block 330, one or more metrics for the genetic variation is identified based on the sequencing data. The one or more metrics may comprise the size of the genetic variation and/or the location of the genetic variation. In some embodiments, the location of the genetic variation is a breakpoint of the genetic variation. In some embodiments, the one or more metrics for the genetic variation may be identified by comparing the sequencing data with data of a reference genome, or a region of the reference genome.

At block 335, a mosaicism ratio (MR) for the genetic variation in the biological sample is determined. The determination of the MR may be based on the sequencing data and/or the one or more metrics. In various embodiments, the MR is calculated by: (a) determining a fraction of nucleic acid having the genetic variation in the circulating cell free nucleic acid based on the sequencing data and/or the one or more metrics, (b) determining a fraction of the fetal nucleic acid in the circulating cell free nucleic acid based on the sequencing data and/or the one or more metrics, and (c) generating the MR using the fraction of (a) to the fraction of (b).

At block 340, the origin of the genetic variation in the biological sample is classified based on the mosaicism ratio and/or the one or more metrics. In some embodiments, the origin of the genetic variation is classified as maternal when the mosaicism ratio is greater than or equal to a threshold. The threshold may be predetermined using a machine-learning training process, and may be adjusted by an authorized practitioner, such as a laboratory director. The threshold may be an empirical threshold based on a statistical analysis. The threshold may depend on the genetic variation. For example, when the genetic variation is a 22q11.2DS, the threshold may be determined to be 3. That is, when the mosaicism ratio for 22q11.2DS of the biological sample is greater than or equal to 3, the origin of the genetic variation is classified to be maternal. In some embodiments, the origin of the genetic variation is classified as fetal when the mosaicism ratio is less than a threshold.

A same threshold may be used for maternal and fetal classification. In some embodiments, the threshold for fetal classification is 3, and when the MR of the biological sample is less than 3, the origin of the genetic variation is classified to be fetal. The threshold for fetal classification may be different from the threshold for maternal. In some embodiments, the threshold for fetal classification is exactly or about 2, 1.9, 1.8, 1.7, 1.6, 1.5, 1.4, 1.3, 1.2, 1.1, or 1. In some embodiments, the classification of the origin or the threshold is independent of the number of fetuses the pregnant woman is carrying at the time the biological sample is obtained. In some embodiments, the threshold for fetal classification or for maternal classification is adjusted based on the number of fetuses the pregnant woman is carrying at the time the biological sample is obtained. The adjustment may be made using a pretrained machine-learning model, or by an authorized practitioner.

In some embodiment, a probability or likelihood of the origin of the genetic variation is predicted at block 235, e.g., 80% that the genetic variation is originated from the maternal nucleic acid. In some embodiment, the probability or likelihood is predicted using a pre-trained machine-learning model based on the MR and/or a predetermined threshold. In some embodiment, the probability or likelihood is affected by the number of fetuses the pregnant woman is carrying at the time the biological sample is obtained, or by the number of fetuses that are suspect to carry the genetic variation. The probability or the likelihood may be further used for downstream applications such as determining whether or not administering a follow-up screening test.

At block 345, a report is generated concerning a recommendation of a follow-up screening based on the one or more metrics and the origin of the genetic variation. The report may further comprise the one or more metrics for the genetic variation, the MR for the genetic variation, the origin of the genetic variation and/or its associated probability or likelihood, partial or all of the sequencing data, available details regarding the pregnant subject, and statistics thereof. In some embodiments, the recommendation is not to perform a specific screening, such as an invasive testing, or a FISH screening. In some embodiments, the recommendation is to perform a specific screening or analysis, such as an invasive testing, or a microarray analysis. The report may further provide a recommendation regarding a specific genomic region or a target sequence that the specific screening is focused on. The specific genomic region or the target sequence is the location/sequence whether the genetic variation occurs. The report may be generated such that it links the genetic variation information to the follow-up recommendations in an intuitive and comprehensible graphical user interface. In some instances, the graphical user interface renders the genetic variation information and the follow-up recommendations with additional patient information/data such as results from other assays or tests performed on the biological sample or a different biological sample.

### E. MACHINES, SOFTWARE AND INTERFACES

Certain processes and methods described herein (e.g., mapping, counting, normalizing, range setting, adjusting, categorizing and/or determining sequence reads, counts, levels and/or profiles) often cannot be performed without a computer, microprocessor, software, module or other machine. Methods described herein typically are computer-implemented methods, and one or more portions of a method sometimes are performed by one or more processors (e.g., microprocessors), computers, systems, apparatuses, or machines (e.g., microprocessor-controlled machine).

Computers, systems, apparatuses, machines and computer program products suitable for use often include, or are utilized in conjunction with, computer readable storage media. Non-limiting examples of computer readable storage media include memory, hard disk, CD-ROM, flash memory device and the like. Computer readable storage media generally are computer hardware, and often are non-transitory computer-readable storage media. Computer readable storage media are not computer readable transmission media, the latter of which are transmission signals per se.

Provided herein are computer readable storage media with an executable program stored thereon, where the program instructs a microprocessor to perform a method described herein. Provided also are computer readable storage media with an executable program module stored thereon, where the program module instructs a microprocessor to perform part of a method described herein. Also provided herein are systems, machines, apparatuses and computer program products that include computer readable storage media with an executable program stored thereon, where the program instructs a microprocessor to perform a method described herein. Provided also are systems, machines and apparatuses that include computer readable storage media with an executable program module stored thereon, where the program module instructs a microprocessor to perform part of a method described herein.

Also provided are computer program products. A computer program product often includes a computer usable medium that includes a computer readable program code embodied therein, the computer readable program code adapted for being executed to implement a method or part of a method described herein. Computer usable media and readable program code are not transmission media (i.e., transmission signals per se). Computer readable program code often is adapted for being executed by a processor, computer, system, apparatus, or machine.

In some embodiments, methods described herein (e.g., quantifying, counting, filtering, normalizing, transforming, clustering and/or determining sequence reads, counts, levels, profiles and/or outcomes) are performed by automated methods. In some embodiments, one or more steps of a method described herein are carried out by a microprocessor and/or computer, and/or carried out in conjunction with memory. In some embodiments, an automated method is embodied in software, modules, microprocessors, peripherals and/or a machine comprising the like, that perform methods described herein. As used herein, software refers to computer readable program instructions that, when executed by a microprocessor, perform computer operations, as described herein.

Sequence reads, counts, levels and/or profiles sometimes are referred to as “data” or “data sets.” In some embodiments, data or data sets can be characterized by one or more features or variables (e.g., sequence based (e.g., GC content, specific nucleotide sequence, the like), function specific (e.g., expressed genes, cancer genes, the like), location based (genome specific, chromosome specific, portion or portion-specific), the like and combinations thereof). In certain embodiments, data or data sets can be organized into a matrix having two or more dimensions based on one or more features or variables. Data organized into matrices can be organized using any suitable features or variables. In certain embodiments, data sets characterized by one or more features or variables sometimes are processed after counting.

Machines, software and interfaces may be used to conduct methods described herein. Using machines, software and interfaces, a user may enter, request, query or determine options for using particular information, programs or processes (e.g., mapping sequence reads, processing mapped data and/or providing an outcome), which can involve implementing statistical analysis algorithms, statistical significance algorithms, statistical algorithms, iterative steps, validation algorithms, and graphical representations, for example. In some embodiments, a data set may be entered by a user as input information, a user may download one or more data sets by suitable hardware media (e.g., flash drive), and/or a user may send a data set from one system to another for subsequent processing and/or providing an outcome (e.g., send sequence read data from a sequencer to a computer system for sequence read mapping; send mapped sequence data to a computer system for processing and yielding an outcome and/or report).

A system typically comprises one or more machines. Each machine comprises one or more of memory, one or more microprocessors, and instructions. Where a system includes two or more machines, some or all of the machines may be located at the same location, some or all of the machines may be located at different locations, all of the machines may be located at one location and/or all of the machines may be located at different locations. Where a system includes two or more machines, some or all of the machines may be located at the same location as a user, some or all of the machines may be located at a location different than a user, all of the machines may be located at the same location as the user, and/or all of the machines may be located at one or more locations different than the user.

A system sometimes comprises a computing machine and a sequencing apparatus or machine, where the sequencing apparatus or machine is configured to receive physical nucleic acid and generate sequence reads, and the computing apparatus is configured to process the reads from the sequencing apparatus or machine. The computing machine sometimes is configured to determine a classification outcome from the sequence reads.

A user may, for example, place a query to software which then may acquire a data set via internet access, and in certain embodiments, a programmable microprocessor may be prompted to acquire a suitable data set based on given parameters. A programmable microprocessor also may prompt a user to select one or more data set options selected by the microprocessor based on given parameters. A programmable microprocessor may prompt a user to select one or more data set options selected by the microprocessor based on information found via the internet, other internal or external information, or the like. Options may be chosen for selecting one or more data feature selections, one or more statistical algorithms, one or more statistical analysis algorithms, one or more statistical significance algorithms, iterative steps, one or more validation algorithms, and one or more graphical representations of methods, machines, apparatuses, computer programs or a non-transitory computer-readable storage medium with an executable program stored thereon.

Systems addressed herein may comprise general components of computer systems, such as, for example, network servers, laptop systems, desktop systems, handheld systems, personal digital assistants, computing kiosks, and the like. A computer system may comprise one or more input means such as a keyboard, touch screen, mouse, voice recognition or other means to allow the user to enter data into the system. A system may further comprise one or more outputs, including, but not limited to, a display screen (e.g., CRT or LCD), speaker, FAX machine, printer (e.g., laser, ink jet, impact, black and white or color printer), or other output useful for providing visual, auditory and/or hardcopy output of information (e.g., outcome and/or report).

In a system, input and output components may be connected to a central processing unit which may comprise among other components, a microprocessor for executing program instructions and memory for storing program code and data. In some embodiments, processes may be implemented as a single user system located in a single geographical site. In certain embodiments, processes may be implemented as a multi-user system. In the case of a multi-user implementation, multiple central processing units may be connected by means of a network. The network may be local, encompassing a single department in one portion of a building, an entire building, span multiple buildings, span a region, span an entire country or be worldwide. The network may be private, being owned and controlled by a provider, or it may be implemented as an internet-based service where the user accesses a web page to enter and retrieve information. Accordingly, in certain embodiments, a system includes one or more machines, which may be local or remote with respect to a user. More than one machine in one location or multiple locations may be accessed by a user, and data may be mapped and/or processed in series and/or in parallel. Thus, a suitable configuration and control may be utilized for mapping and/or processing data using multiple machines, such as in local network, remote network and/or “cloud” computing platforms.

A system can include a communications interface in some embodiments. A communications interface allows for transfer of software and data between a computer system and one or more external devices. Non-limiting examples of communications interfaces include a modem, a network interface (such as an Ethernet card), a communications port, a PCMCIA slot and card, and the like. Software and data transferred via a communications interface generally are in the form of signals, which can be electronic, electromagnetic, optical and/or other signals capable of being received by a communications interface. Signals often are provided to a communications interface via a channel. A channel often carries signals and can be implemented using wire or cable, fiber optics, a phone line, a cellular phone link, an RF link and/or other communications channels. Thus, in an example, a communications interface may be used to receive signal information that can be detected by a signal detection module.

Data may be input by a suitable device and/or method, including, but not limited to, manual input devices or direct data entry devices (DDEs). Non-limiting examples of manual devices include keyboards, concept keyboards, touch sensitive screens, light pens, mouse, tracker balls, joysticks, graphic tablets, scanners, digital cameras, video digitizers and voice recognition devices. Non-limiting examples of DDEs include bar code readers, magnetic strip codes, smart cards, magnetic ink character recognition, optical character recognition, optical mark recognition, and turnaround documents.

In some embodiments, output from a sequencing apparatus or machine may serve as data that can be input via an input device. In certain embodiments, mapped sequence reads may serve as data that can be input via an input device. In certain embodiments, nucleic acid fragment size (e.g., length) may serve as data that can be input via an input device. In certain embodiments, output from a nucleic acid capture process (e.g., genomic region origin data) may serve as data that can be input via an input device. In certain embodiments, a combination of nucleic acid fragment size (e.g., length) and output from a nucleic acid capture process (e.g., genomic region origin data) may serve as data that can be input via an input device. In certain embodiments, simulated data is generated by an in silico process and the simulated data serves as data that can be input via an input device. The term “in silico” refers to research and experiments performed using a computer. In silico processes include, but are not limited to, mapping sequence reads and processing mapped sequence reads according to processes described herein.

A system may include software useful for performing a process or part of a process described herein, and software can include one or more modules for performing such processes (e.g., sequencing module, logic processing module, data display organization module). The term “software” refers to computer readable program instructions that, when executed by a computer, perform computer operations. Instructions executable by the one or more microprocessors sometimes are provided as executable code, that when executed, can cause one or more microprocessors to implement a method described herein. A module described herein can exist as software, and instructions (e.g., processes, routines, subroutines) embodied in the software can be implemented or performed by a microprocessor. For example, a module (e.g., a software module) can be a part of a program that performs a particular process or task. The term “module” refers to a self-contained functional unit that can be used in a larger machine or software system. A module can comprise a set of instructions for carrying out a function of the module. A module can transform data and/or information. Data and/or information can be in a suitable form. For example, data and/or information can be digital or analogue. In certain embodiments, data and/or information sometimes can be packets, bytes, characters, or bits. In some embodiments, data and/or information can be any gathered, assembled or usable data or information. Non-limiting examples of data and/or information include a suitable media, pictures, video, sound (e.g. frequencies, audible or non-audible), numbers, constants, a value, objects, time, functions, instructions, maps, references, sequences, reads, mapped reads, levels, ranges, thresholds, signals, displays, representations, or transformations thereof. A module can accept or receive data and/or information, transform the data and/or information into a second form, and provide or transfer the second form to an machine, peripheral, component or another module. A module can perform one or more of the following non-limiting functions: mapping sequence reads, providing counts, assembling portions, providing or determining a level, providing a count profile, normalizing (e.g., normalizing reads, normalizing counts, and the like), providing a normalized count profile or levels of normalized counts, comparing two or more levels, providing uncertainty values, providing or determining expected levels and expected ranges (e.g., expected level ranges, threshold ranges and threshold levels), providing adjustments to levels (e.g., adjusting a first level, adjusting a second level, adjusting a profile of a chromosome or a part thereof, and/or padding), providing identification (e.g., identifying a copy number alteration, genetic variation/genetic alteration or aneuploidy), categorizing, plotting, and/or determining an outcome, for example. A microprocessor can, in certain embodiments, carry out the instructions in a module. In some embodiments, one or more microprocessors are required to carry out instructions in a module or group of modules. A module can provide data and/or information to another module, machine or source and can receive data and/or information from another module, machine or source.

A computer program product sometimes is embodied on a tangible computer-readable medium, and sometimes is tangibly embodied on a non-transitory computer-readable medium. A module sometimes is stored on a computer readable medium (e.g., disk, drive) or in memory (e.g., random access memory). A module and microprocessor capable of implementing instructions from a module can be located in a machine or in a different machine. A module and/or microprocessor capable of implementing an instruction for a module can be located in the same location as a user (e.g., local network) or in a different location from a user (e.g., remote network, cloud system). In embodiments in which a method is carried out in conjunction with two or more modules, the modules can be located in the same machine, one or more modules can be located in different machine in the same physical location, and one or more modules may be located in different machines in different physical locations.

A machine, in some embodiments, comprises at least one microprocessor for carrying out the instructions in a module. Sequence read quantifications (e.g., counts) sometimes are accessed by a microprocessor that executes instructions configured to carry out a method described herein. Sequence read quantifications that are accessed by a microprocessor can be within memory of a system, and the counts can be accessed and placed into the memory of the system after they are obtained. In some embodiments, a machine includes a microprocessor (e.g., one or more microprocessors) which microprocessor can perform and/or implement one or more instructions (e.g., processes, routines and/or subroutines) from a module. In some embodiments, a machine includes multiple microprocessors, such as microprocessors coordinated and working in parallel. In some embodiments, a machine operates with one or more external microprocessors (e.g., an internal or external network, server, storage device and/or storage network (e.g., a cloud)). In some embodiments, a machine comprises a module (e.g., one or more modules). A machine comprising a module often is capable of receiving and transferring one or more of data and/or information to and from other modules.

In certain embodiments, a machine comprises peripherals and/or components. In certain embodiments, a machine can comprise one or more peripherals or components that can transfer data and/or information to and from other modules, peripherals and/or components. In certain embodiments, a machine interacts with a peripheral and/or component that provides data and/or information. In certain embodiments, peripherals and components assist a machine in carrying out a function or interact directly with a module. Non-limiting examples of peripherals and/or components include a suitable computer peripheral, I/O or storage method or device including but not limited to scanners, printers, displays (e.g., monitors, LED, LCT or CRTs), cameras, microphones, pads (e.g., ipads, tablets), touch screens, smart phones, mobile phones, USB I/O devices, USB mass storage devices, keyboards, a computer mouse, digital pens, modems, hard drives, jump drives, flash drives, a microprocessor, a server, CDs, DVDs, graphic cards, specialized I/O devices (e.g., sequencers, photo cells, photo multiplier tubes, optical readers, sensors, etc.), one or more flow cells, fluid handling components, network interface controllers, ROM, RAM, wireless transfer methods and devices (Bluetooth, WiFi, and the like,), the world wide web (www), the internet, a computer and/or another module.

Software often is provided on a program product containing program instructions recorded on a computer readable medium, including, but not limited to, magnetic media including floppy disks, hard disks, and magnetic tape; and optical media including CD-ROM discs, DVD discs, magneto-optical discs, flash memory devices (e.g., flash drives), RAM, floppy discs, the like, and other such media on which the program instructions can be recorded. In online implementation, a server and web site maintained by an organization can be configured to provide software downloads to remote users, or remote users may access a remote system maintained by an organization to remotely access software. Software may obtain or receive input information. Software may include a module that specifically obtains or receives data (e.g., a data receiving module that receives sequence read data and/or mapped read data) and may include a module that specifically processes the data (e.g., a processing module that processes received data (e.g., filters, normalizes, provides an outcome and/or report). The terms “obtaining” and “receiving” input information refers to receiving data (e.g., sequence reads, mapped reads) by computer communication means from a local, or remote site, human data entry, or any other method of receiving data. The input information may be generated in the same location at which it is received, or it may be generated in a different location and transmitted to the receiving location. In some embodiments, input information is modified before it is processed (e.g., placed into a format amenable to processing (e.g., tabulated)).

Software can include one or more algorithms in certain embodiments. An algorithm may be used for processing data and/or providing an outcome or report according to a finite sequence of instructions. An algorithm often is a list of defined instructions for completing a task. Starting from an initial state, the instructions may describe a computation that proceeds through a defined series of successive states, eventually terminating in a final ending state. The transition from one state to the next is not necessarily deterministic (e.g., some algorithms incorporate randomness). By way of example, and without limitation, an algorithm can be a search algorithm, sorting algorithm, merge algorithm, numerical algorithm, graph algorithm, string algorithm, modeling algorithm, computational genometric algorithm, combinatorial algorithm, machine learning algorithm, cryptography algorithm, data compression algorithm, parsing algorithm and the like. An algorithm can include one algorithm or two or more algorithms working in combination. An algorithm can be of any suitable complexity class and/or parameterized complexity. An algorithm can be used for calculation and/or data processing, and in some embodiments, can be used in a deterministic or probabilistic/predictive approach. An algorithm can be implemented in a computing environment by use of a suitable programming language, non-limiting examples of which are C, C++, Java, Perl, Python, Fortran, and the like. In some embodiments, an algorithm can be configured or modified to include margin of errors, statistical analysis, statistical significance, and/or comparison to other information or data sets (e.g., applicable when using a neural net or clustering algorithm).

In certain embodiments, several algorithms may be implemented for use in software. These algorithms can be trained with raw data in some embodiments. For each new raw data sample, the trained algorithms may produce a representative processed data set or outcome. A processed data set sometimes is of reduced complexity compared to the parent data set that was processed. Based on a processed set, the performance of a trained algorithm may be assessed based on sensitivity and specificity, in some embodiments. An algorithm with the highest sensitivity and/or specificity may be identified and utilized, in certain embodiments.

In certain embodiments, simulated (or simulation) data can aid data processing, for example, by training an algorithm or testing an algorithm. In some embodiments, simulated data includes hypothetical various samplings of different groupings of sequence reads. Simulated data may be based on what might be expected from a real population or may be skewed to test an algorithm and/or to assign a correct classification. Simulated data also is referred to herein as “virtual” data. Simulations can be performed by a computer program in certain embodiments. One possible step in using a simulated data set is to evaluate the confidence of identified results, e.g., how well a random sampling matches or best represents the original data. One approach is to calculate a probability value (p-value), which estimates the probability of a random sample having better score than the selected samples. In some embodiments, an empirical model may be assessed, in which it is assumed that at least one sample matches a reference sample (with or without resolved variations). In some embodiments, another distribution, such as a Poisson distribution for example, can be used to define the probability distribution.

A system may include one or more microprocessors in certain embodiments. A microprocessor can be connected to a communication bus. A computer system may include a main memory, often random access memory (RAM), and can also include a secondary memory. Memory in some embodiments comprises a non-transitory computer-readable storage medium. Secondary memory can include, for example, a hard disk drive and/or a removable storage drive, representing a floppy disk drive, a magnetic tape drive, an optical disk drive, memory card and the like. A removable storage drive often reads from and/or writes to a removable storage unit. Non-limiting examples of removable storage units include a floppy disk, magnetic tape, optical disk, and the like, which can be read by and written to by, for example, a removable storage drive. A removable storage unit can include a computer-usable storage medium having stored therein computer software and/or data.

A microprocessor may implement software in a system. In some embodiments, a microprocessor may be programmed to automatically perform a task described herein that a user could perform. Accordingly, a microprocessor, or algorithm conducted by such a microprocessor, can require little to no supervision or input from a user (e.g., software may be programmed to implement a function automatically). In some embodiments, the complexity of a process is so large that a single person or group of persons could not perform the process in a timeframe short enough for determining the presence or absence of a genetic variation or genetic alteration.

In some embodiments, secondary memory may include other similar means for allowing computer programs or other instructions to be loaded into a computer system. For example, a system can include a removable storage unit and an interface device. Non-limiting examples of such systems include a program cartridge and cartridge interface (such as that found in video game devices), a removable memory chip (such as an EPROM, or PROM) and associated socket, and other removable storage units and interfaces that allow software and data to be transferred from the removable storage unit to a computer system.

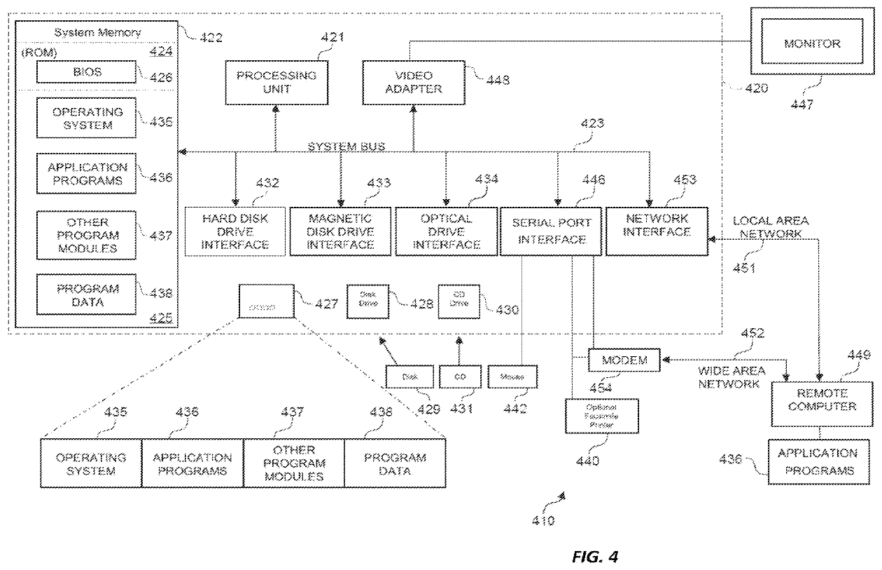

FIG. 4 illustrates a non-limiting example of a computing environment 410 in which various systems, methods, algorithms, and data structures described herein may be implemented. The computing environment 410 is only one example of a suitable computing environment and is not intended to suggest any limitation as to the scope of use or functionality of the systems, methods, and data structures described herein. Neither should computing environment 410 be interpreted as having any dependency or requirement relating to any one or combination of components illustrated in computing environment 410. A subset of systems, methods, and data structures shown in FIG. 4 can be utilized in certain embodiments. Systems, methods, and data structures described herein are operational with numerous other general purpose or special purpose computing system environments or configurations. Examples of known computing systems, environments, and/or configurations that may be suitable include, but are not limited to, personal computers, server computers, thin clients, thick clients, hand-held or laptop devices, multiprocessor systems, microprocessor-based systems, set top boxes, programmable consumer electronics, network PCs, minicomputers, mainframe computers, distributed computing environments that include any of the above systems or devices, and the like.

The computing environment 410 of FIG. 4 includes a general-purpose computing device in the form of a computer 420, including a processing unit 421, a system memory 422, and a system bus 423 that operatively couples various system components including the system memory 422 to the processing unit 421. There may be only one or there may be more than one processing unit 421, such that the processor of computer 420 includes a single central-processing unit (CPU), or a plurality of processing units, commonly referred to as a parallel processing environment. The computer 420 may be a conventional computer, a distributed computer, or any other type of computer.

The system bus 423 may be any of several types of bus structures including a memory bus or memory controller, a peripheral bus, and a local bus using any of a variety of bus architectures. The system memory may also be referred to as simply the memory and includes read only memory (ROM) 424 and random access memory (RAM). A basic input/output system (BIOS) 426, containing the basic routines that help to transfer information between elements within the computer 420, such as during start-up, is stored in ROM 424. The computer 420 may further include a hard disk drive 427 for reading from and writing to a hard disk, not shown, a magnetic disk drive 428 for reading from or writing to a removable magnetic disk 429, and an optical disk drive 430 for reading from or writing to a removable optical disk 431 such as a CD ROM or other optical media.

The hard disk drive 427, magnetic disk drive 428, and optical disk drive 430 are connected to the system bus 423 by a hard disk drive interface 432, a magnetic disk drive interface 433, and an optical disk drive interface 434, respectively. The drives and their associated computer-readable media provide nonvolatile storage of computer-readable instructions, data structures, program modules and other data for the computer 420. Any type of computer-readable media that can store data that is accessible by a computer, such as magnetic cassettes, flash memory cards, digital video disks, Bernoulli cartridges, random access memories (RAMs), read only memories (ROMs), and the like, may be used in the operating environment.

A number of program modules may be stored on the hard disk, magnetic disk 429, optical disk 431, ROM 424, or RAM, including an operating system 435, one or more application programs 436, other program modules 437, and program data 438. A user may enter commands and information into the computer 420 through input devices such as a keyboard 440 and pointing device 442. Other input devices (not shown) may include a microphone, joystick, game pad, satellite dish, scanner, or the like. These and other input devices are often connected to the processing unit 421 through a serial port interface 446 that is coupled to the system bus, but may be connected by other interfaces, such as a parallel port, game port, or a universal serial bus (USB). A monitor 447 or other type of display device is also connected to the system bus 423 via an interface, such as a video adapter 448. In addition to the monitor, computers typically include other peripheral output devices (not shown), such as speakers and printers.

The computer 420 may operate in a networked environment using logical connections to one or more remote computers, such as remote computer 449. These logical connections may be achieved by a communication device coupled to or a part of the computer 420, or in other manners. The remote computer 449 may be another computer, a server, a router, a network PC, a client, a peer device or other common network node, and typically includes many or all of the elements described above relative to the computer 420, although only a memory storage device 425 has been illustrated in FIG. 4. The logical connections depicted in FIG. 4 include a local-area network (LAN) 451 and a wide-area network (WAN) 452. Such networking environments are commonplace in office networks, enterprise-wide computer networks, intranets and the Internet, which all are types of networks.

When used in a LAN-networking environment, the computer 420 is connected to the local-area network (LAN) 451 through a network interface or adapter 453, which is one type of communications device. When used in a WAN-networking environment, the computer 420 often includes a modem 454, a type of communications device, or any other type of communications device for establishing communications over the wide-area network (WAN) 452. The modem 454, which may be internal or external, is connected to the system bus 423 via the serial port interface 446. In a networked environment, program modules depicted relative to the computer 420, or portions thereof, may be stored in the remote memory storage device. It is appreciated that the network connections shown are non-limiting examples and other communications devices for establishing a communications link between computers may be used.

### F. EXAMPLES

The examples set forth below illustrate certain embodiments and do not limit the technology.

### Example 1: A Retrospective Study with Screen-Positive 22q11.2 NIPS Results on a Massively Parallel Sequencing (MPS) Platform

### a) Introduction

Since the introduction of cell-free DNA (cfDNA) screening, significant progress has been made in the area of non-invasive prenatal screening. Current screening is moving beyond traditional cfDNA for the common aneuploidies, expanding in scope to genome-wide copy number variants and single gene disorders. Another area of expansion for cfDNA screening is the detection of select microdeletion syndromes. Of particular interest is screening for 22q11.2 deletion syndrome (22q11.2DS), a condition with a reported prevalence of 1 in 1,000 in unselected fetuses or 1 in 3000-6,000 live births.

Also known as DiGeorge syndrome (OMIM #188400) or velocardiofacial syndrome (VCFS, OMIM #192430), 22q11.2DS is associated with a wide spectrum of anomalies, with cardiac defects, immune system dysfunction, oral clefting, hypocalcemia, developmental delays, and behavioral complications being among the most commonly reported features. However, some of these features cannot be ascertained during the prenatal or even the neonatal period (e.g. developmental delays, immune dysfunction, behavioral complications, etc.) although cardiac anomalies, oral clefting, polyhydramnios, renal abnormalities, and skeletal abnormalities are among the findings that may be identified during routine ultrasonography. CfDNA screening for 22q11.2DS became clinically available, yet most professional societies have yet to support routine screening for this condition during pregnancy due to a lack of clinical data and the perception of a reduced positive predictive value (PPV) associated with a positive screening result, ranging from 18% to greater than 97%. Recently, the American College of Medical Genetics (ACMG) ‘suggests that screening for 22q11.2 deletion syndrome be offered to all patients’ as a conditional recommendation based on moderate certainty of evidence. Prenatal identification of 22q11.2DS allows parents to make informed reproductive choices and may have benefit for both perinatal management and maternal health, and may help to avoid a potential ‘diagnostic odyssey’ for an affected child. Furthermore, 22q11.2DS often occurs de novo, is not associated with maternal age, and affected fetuses may or may not present with ultrasound findings; these features suggest that many cases of 22q11.2DS may not otherwise be identified in the prenatal or neonatal period via ultrasound or routine assessment of the patient's family or obstetric history.

Screening for 22q11.2DS by cfDNA is accompanied by inherent biological and technical challenges. A variety of deletion sizes have been reported with 22q11.2DS owing to the low copy number repeats (LCRs), labeled A through D, in the area. Approximately 85% of patients have a ~2.54 Mb sized deletion, which had been frequently described as a ‘3 Mb’ deletion, from A-D LCRs (10). The remaining ~15% have smaller atypical or nested deletions (for example, involving the A-B LCRs or C-D LCRs) which may be associated with a milder phenotype and reduced penetrance. This region is particularly susceptible to meiotic error due to the significant homology between these LCRs; non-allelic homologous recombination leads to both the ‘3 Mb’ deletion as well as the smaller, nested deletions. Given the size of 22q11.2 deletions, even the typical ‘3 Mb’ deletion will be below the resolution of detection by routine prenatal chromosome analysis; diagnostic confirmation of 22q11.2 deletions typically requires microarray and/or fluorescent in situ hybridization (FISH). Additionally, common FISH probes for detection of 22q11.2 deletions used by diagnostic laboratories may include N25, TUPLE1/HIRA and TBX1. These probes generally hybridize between the A-B LCRs, a region included in the typical ‘3 Mb’ deletion. However, use of these FISH probes for the detection of atypical nested deletions may have limited clinical utility as the deletion may be proximal or distal to the FISH probes used.

Approximately 10% of affected individuals inherit the deletion from an affected parent, although this percentage is significantly higher (60%) for nested/smaller atypical deletions. CfDNA screening utilizes maternal plasma containing both fetal (placental) and maternal cfDNA, therefore, presence of a maternal CNV in a region of interest precludes fetal assessment of that particular region. Even though fetal assessment using cfDNA may not be possible when a maternal CNV is suspected, detection and reporting of a suspected maternal 22q11.2 deletion suggests a 50% risk for an affected fetus. These factors underline both the benefits and limitations of this screening test, and support the role of diagnostic confirmation as the next most reasonable step. Any patient with a screen positive result should be referred for genetic counseling, with a discussion on the option of diagnostic testing.

The current study addresses these clinical and laboratory considerations following a screen-positive cfDNA result by analyzing a cohort of 307 samples where a 22q11.2 deletion (either fetal or suspected maternal) was detected via cfDNA screening at a single commercial laboratory, including an analysis of observed PPVs in a retrospective cohort of patients that pursued diagnostic testing. This study also examines how massively parallel sequencing (MPS)-based cfDNA screening is capable of predicting maternal versus fetal events involving the 22q11.2 region, the implications of the presence of ultrasound findings and/or maternal phenotype, the predicted sizes and locations of 22q11.2 deletions on cfDNA as compared to predicted size/location on diagnostic testing in the cohort, and considerations for follow-up diagnostic testing following a screen positive result.

### b) Materials and Methods

Maternal blood samples were submitted for cell-free DNA screening with analysis of 22q11.2 deletion syndrome using either ‘traditional’ cfDNA screening with microdeletion analysis (MaterniT® 21 PLUS with Enhanced Sequencing Series) or genome-wide cfDNA screening (MaterniTR GENOME) as selected by the ordering provider. MaterniT® 21 PLUS screens for trisomies of chromosomes 21, 18, and 13 with the option of expanded content (sex chromosome aneuploidies, and the Enhanced Sequencing Series which includes trisomies 16 and 22 as well as microdeletions associated with 1p36 deletion, Wolf-Hirschhorn, Cri-du-chat, Langer-Giedion, Jacobsen, Prader-Willi, Angelman, and DiGeorge syndromes). MaterniTR GENOME screens for aneuploidy of any chromosome, CNVs ≥7 Mb in size as well as the select microdeletions <7 Mb in size mentioned above. During the time frame, nearly 850,000 samples were screened for 22q11.2 deletion syndrome across both assays. Blood samples were subjected to DNA extraction, library preparation, and genome-wide MPS. For cases submitted for genome-wide analysis, sequencing data were analyzed using a proprietary algorithm to detect aneuploidies and other subchromosomal events known in the art. Pretest and posttest counseling and informed consent were the responsibility of the clinicians ordering the testing.

All clinical cfDNA specimens which detected suspected fetal and suspected maternal 22q11.2 deletions were compiled, along with all available diagnostic outcomes. Diagnostic outcomes were obtained from two sources. First, outcome information was collected, when available, from the ordering provider. Second, positive cfDNA samples were cross-referenced with diagnostic results (FISH, microarray, and karyotype) from chorionic villus, amniocentesis, neonatal and/or maternal peripheral blood, and products of conception specimens during a corresponding timeframe. The process of consolidation and comparison of data across the datasets (cfDNA results and microarray results) was approved by Aspire IRB under clinical protocol SCMM-RND-402. For a cfDNA sample to be considered a match to a microarray specimen, the diagnostic and screening results were required to have identical patient identifiers (name and date of birth), and the collection date for the diagnostic test had to be within 90 days of the patient's cfDNA screening date. Cases outside the 90-day window were adjudicated to ensure the screening and diagnostic results were from the same pregnancy. However, matched diagnostic testing on a maternal blood specimen could have occurred at any time and was not limited to the 90-day window. Because postnatal testing on the neonate is typically under a different set of identifiers (typically the name and birth date of the neonate), matching of those samples back to the cfDNA specimen was not possible in most cases. Obstetric pregnancy outcome and postnatal follow-up were rarely available and thus are not analyzed in the study. A cfDNA result was classified as a ‘true positive’ for 22q11.2 deletion syndrome when the deletion was confirmed via FISH testing and/or microarray analysis. A ‘false positive’ classification was assigned when the abnormal screening result was not confirmed by diagnostic testing. Cases were considered to have ‘incomplete’ diagnostic testing when only FISH or karyotype were normal, with no microarray ordered, or when a maternal deletion was suspected based on the cfDNA sequencing data and only fetal testing was performed, or vice versa (fetal deletion was suspected based on the cfDNA sequencing data and only maternal testing was performed). Such cases were not treated as false positives, as the completed testing was inadequate to rule out the presence of a 22q11.2 microdeletion. However, PPV calculations were performed in multiple ways, including a version where these were treated as hypothetical false positives for the most conservative estimation of PPV.

Secondary to bioinformatics and assay enhancements, additional data for screen-positive 22q11.2 deletion specimens included the estimated deletion size, estimated breakpoints, and the event specific fraction on cfDNA allowing for the calculation of mosaicism ratio. Mosaicism ratio (MR) is a laboratory metric derived by dividing the fraction of cfDNA associated with the abnormal event by the overall fetal fraction of the specimen. Cases with a disproportionally high MR (in consideration with other sequencing metrics, such as a considerably elevated z-score), were assigned as ‘likely maternal’ for reporting by the laboratory director. A proprietary algorithm is used to flag events that are ‘likely maternal,’ but the laboratory director has the final discretion to determine how to report the event in context of the rest of the sequencing data. In these circumstances, patient results were reported as screen positive for 22q11.2 DS with an additional ‘likely maternal’ comment and also noted assessment of fetal status for the 22q11.2 region was precluded. Cases without this additional comment on the patient report were designated ‘suspected fetal’ for the purpose of the current study.

Maternal demographics and indications for testing were recorded as provided by the ordering clinician at the time of testing on the test requisition form (TRF). TRFs were reviewed for additional details about ultrasound findings and/or family history, where indicated; the laboratory's clinical database used for documenting client discussions and tracking outcomes was also reviewed for additional notes from the provider about ultrasound findings, family history, pregnancy outcomes, and any other relevant case details. These demographics and details about test indications were compiled and analyzed.

Study data was statistically described using counts, rates, and measures of central tendency. Positive predictive value with 95% confidence interval was calculated using the VassarStats Website for Statistical Computation. Deletion sizes and mosaicism ratios were compared using Wilcoxon rank-sum tests. A Fisher's exact test was used to test for an association between ultrasound findings and fetal diagnostic testing. Concordance between cfDNA-predicted deletion size and microarray-confirmed deletion size was plotted and fitted with a linear regression line. For all calculations, p-values less than 0.05 were considered statistically significant. Statistical analyses and generation of plots and figures were performed using R and the publicly available packages.

Collection of outcomes was approved by AspireIRB under clinical protocol SCMM-RND-402 and all clinical data was de-identified. Informed consent was not required as AspireIRB declared that this research meets the requirements for a waiver of consent under 45 CFR 46 116(f).

### c) Results

### (1) Study Cohort

There were 307 cases with 22q11.2 deletions reported either as suspected maternal (precluding fetal assessment) (57.7%, n=177) or suspected fetal (42.3%, n=130) from cfDNA screening through August 2021. Most cases were identified via the standard cfDNA assay with select microdeletions opted-in (64.2%, n=197), while the remainder were identified via the genome-wide assay (35.8%, n=110). Nearly all cases were singleton pregnancies (99.3%, n=305), with 2 cases of twin pregnancies (0.7%). The median maternal age was 29.0 years (range 15-45 years) with a median gestational age of 19 weeks (range 9-36 weeks). The median fetal fraction was 10.48% (range 2.96%-41.18).

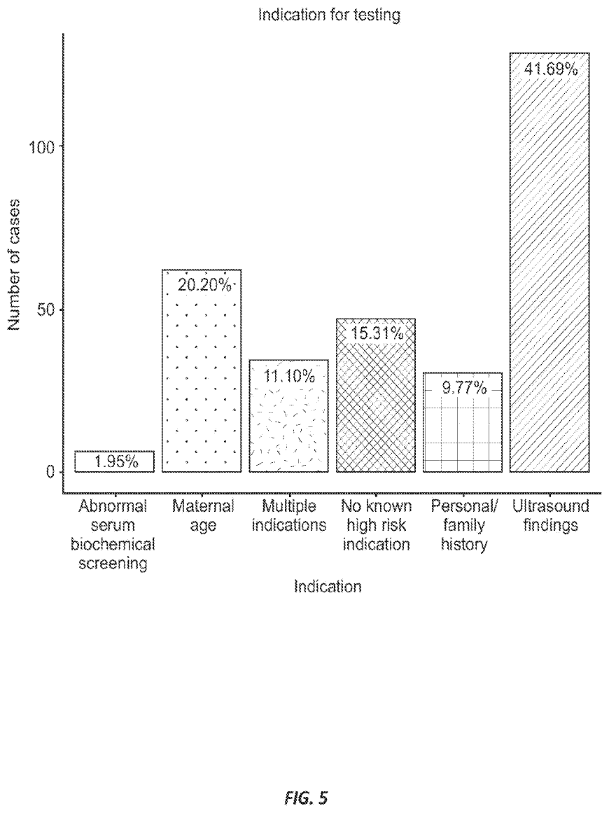

FIG. 5 shows the indications for testing. The most common indication for testing was ultrasound findings (41.7%, n=128), followed by maternal age (20.2%, n=62) and no known high-risk indication (15.3%, n=47). Of note, 34 cases were referred with multiple indications for testing; 23 of those included ultrasound findings as one of the reasons. When outcomes were collected, an additional 6 cases were noted to later have ultrasound findings in the outcome relayed by the provider or noted on the TRF submitted with a diagnostic specimen. Ultimately, 157 cases (51.1%) had an ultrasound finding reported either at the time of testing or at some point during the pregnancy. The details of the ultrasound findings, where available, are further discussed later. For 36/177 of the suspected maternal cases, notes or comments about maternal phenotype (or lack thereof) potentially associated with 22q11.2DS were available; a maternal phenotype was noted in 24 cases and absence of any phenotype was noted in 12 cases.

### (2) CfDNA Results

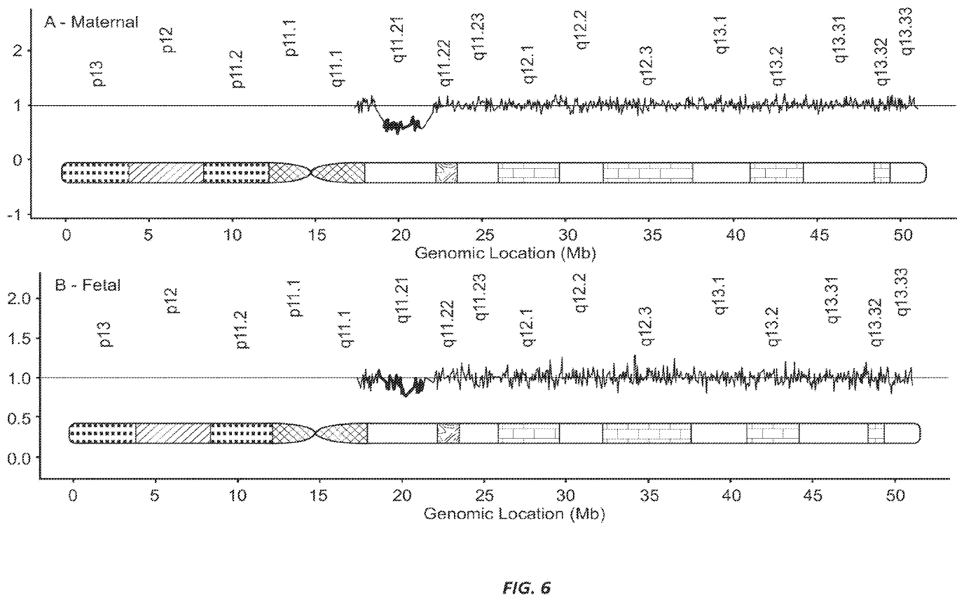

Of the 307 suspected deletions, 177 were suspected to be maternal in origin, while the remaining 130 appeared to be fetal in origin based on the cfDNA sequencing data. FIG. 6 shows an example of the sequencing data for both a suspected maternal deletion and fetal deletion in the region of 22q11.2.

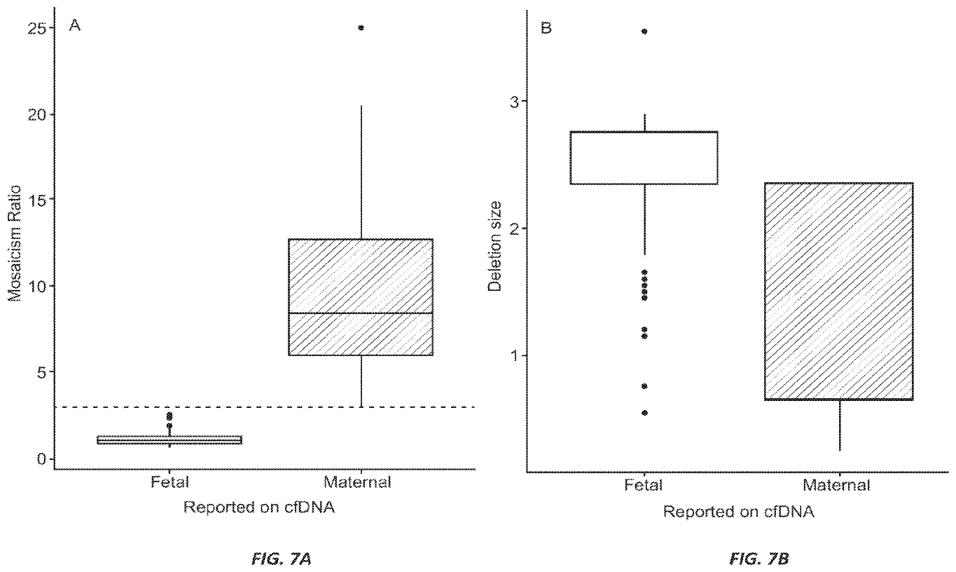

A mosaicism ratio (comparing the fraction of cfDNA harboring the 22q11.2 deletion to the overall fetal fraction) was available for 241 cases (early assay versions did not include this metric). FIG. 7A shows the near-complete separation of MRs between the suspected fetal and suspected maternal cases, with a statistically significant difference in the MR for the suspected fetal versus suspected maternal cases (p<2.2e−16). MR in maternal cases was nearly always above 3 and MR in fetal cases was nearly always less than 3, with median MR in suspected fetal cases of 1.023 and median MR in suspected maternal cases of 8.354.

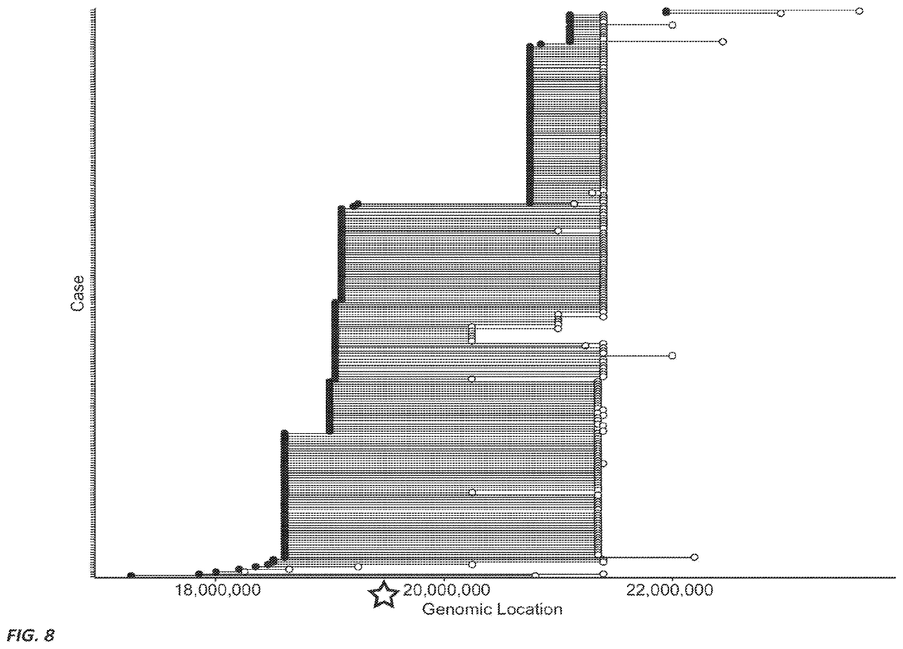

The cfDNA sequencing data provided an estimated deletion size and predicted breakpoints for 237 cases. (Early version of the assay did not predict deletion sizes and breakpoints). Table 1 shows the details of the predicted deletion sizes from cfDNA overall, as well as for suspected maternal deletions and suspected fetal deletions. FIG. 8 shows a plot of the start and end breakpoints predicted from cfDNA, with groupings of frequent deletions of similar sizes, the common, larger deletions toward the bottom of the figure and the smaller nested/atypical deletions toward the top. The star on the x-axis indicates the approximate location of most commercially available FISH probes (N25, TUPLE1/HIRA, TBX1). There was a statistically significant difference (p<2.2e−16) in the predicted sizes between the suspected maternal deletions and suspected fetal deletions. Fetal deletions tended to be larger, while maternal deletions tended to be smaller. FIG. 7B shows a plot of the estimated deletion sizes by suspected maternal versus fetal origin from cfDNA.

TABLE 1
Estimated 22q11.2 deletion sizes from cfDNA (Mb = megabases)

| | Median | IQR | Range |
|---|---|---|---|
| | (Mb) | (Mb) | (Mb) |
| All deletions (n = 237) | 2.35 | 0.65-2.35 | 0.25-3.55 |
| Suspected fetal (n = 99) | 2.75 | 2.35-2.75 | 0.55-3.55 |
| Suspected maternal (n = 138) | 0.65 | 0.65-2.35 | 0.25-2.35 |

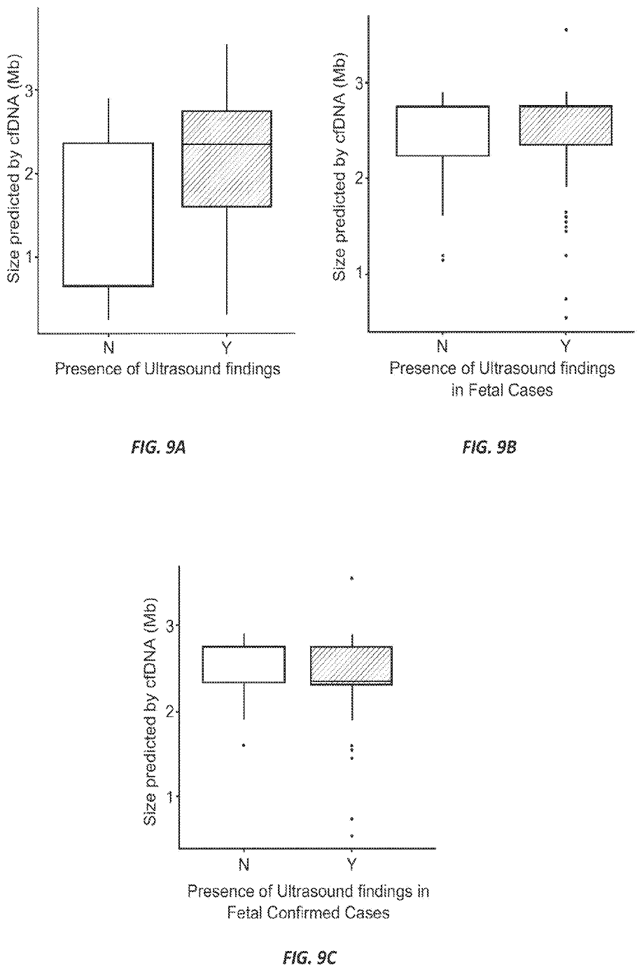

Deletion sizes from cfDNA were then evaluated in the context of ultrasound findings as shown in FIGS. 9A-9C. When suspected maternal cases were removed (which as noted, tend to be smaller), and only suspected fetal cases were analyzed (FIG. 9B, there was no significant different in the size of deletions with and without ultrasound findings (p=0.6687). Analysis of only confirmed fetal cases (FIG. 9C) was also not significant (p=0.2588). Initial analysis of the overall cohort (FIG. 9A), before removal of the suspected maternal cases, showed the appearance of a significant difference in deletion size (p=6.533e−10), which was clearly being skewed by the maternal cases.

Based on the overall cohort there appeared to be a significant difference in the size of the deletions in cases with and without ultrasound findings (p=6.533e−10); however, when the suspected maternal cases (which as noted above, tend to be smaller) were removed, and only suspected fetal cases were analyzed, there was no significant difference (p=0.6687).

### (3) Diagnostic Testing and Positive Predictive Value

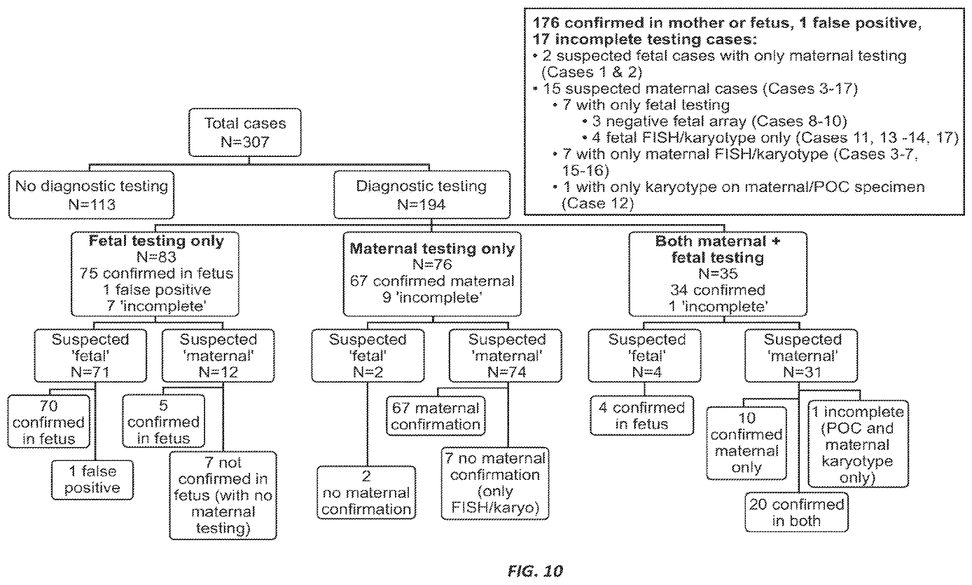

There were 194 cases with a diagnostic test result available (63.2%). Of the 194 cases with diagnostic testing results available, 83 had only fetal testing, 76 had only maternal testing, and 35 had testing for both. Cases with fetal ultrasound findings were significantly more likely (3.45×) to have a fetal diagnostic test than cases without ultrasound (p=3.889e−07, OR=3.45, 95% CI: 2.07-5.83). There were 17 cases that were considered to have incomplete diagnostic testing, such as only karyotype; the details of these 17 cases are described in Table 2. This leaves 177 cases with complete diagnostic testing for calculation of positive predictive values (PPV). If all 177 cases were considered for a PPV calculation (both suspected maternal (n=102) and fetal (n=75), with confirmation on either fetal testing or maternal testing), the PPV is 99.4% (95% CI: 96.4%-99.9%). If only fetal cases are considered (n=75; suspected fetal cases with fetal testing available) the PPV of the assay is 98.7% (95% CI: 91.8%-99.9%). For the most conservative estimate of PPV in the cases with diagnostic testing, if the 17 ‘incomplete’ cases were all considered ‘false positives,’ the PPV is 90.7% (95% CI: 85.5%-94.2%). Table 3 summarizes these PPV calculations. FIG. 10 shows a flow chart that summarizes the diagnostic testing for the 194 cases.

TABLE 2
Details of the ‘incomplete’ testing cases

| | cfDNA details | Diagnostic testing details | Notes |
|---|---|---|---|
| 1 | RI: Ultrasound finding, | Maternal array and karyotype | Heart defect on ultrasound |
| | testing at 21 weeks; | only (both negative); no fetal | |
| | Suspected fetal deletion; MR | testing | |
| | and cfDNA size NA | | |
| 2 | RI: Ultrasound finding, | Parental arrays only (both | TOF, polyhydramnios, and absent |
| | testing at 25 weeks, | negative); no fetal testing | pulmonary valve on ultrasound |
| | Suspected fetal deletion; MR | | |
| | and cfDNA size NA | | |
| 3 | RI: Maternal age, testing at | No fetal testing; maternal testing | Atypical deletion predicted |
| | 21 weeks, Suspected | via FISH only (negative), no | |
| | maternal deletion; MR and | microarray | |
| | cfDNA size NA | | |
| 4 | RI: No known high-risk | No fetal testing; maternal FISH | Provider noted TUPLE1 probe was used; |
| | indication, testing at 16 | and karyotype only (negative), | HIRA gene not included in region |
| | weeks, Suspected maternal | no microarray | seen on cfDNA |
| | deletion; MR 13.448, 0.55 | | |
| | Mb deletion predicted | | |
| 5 | RI: No known high-risk | No fetal testing; maternal FISH | Atypical deletion predicted |
| | indication, testing at 11 | only (negative), no microarray | |
| | weeks, Suspected maternal | | |
| | deletion; MR 14.21, 0.65 Mb | | |
| | deletion predicted | | |
| 6 | RI: Maternal age, testing at | No fetal testing; maternal FISH | Atypical deletion predicted |
| | 12 weeks, Suspected | only (negative), no microarray | |
| | maternal deletion; MR | | |
| | 10.162, 0.65 Mb deletion | | |
| | predicted | | |
| 7 | RI: No known high-risk | No fetal testing; maternal blood | Atypical deletion predicted |
| | indication, testing at 12 | karyotype only (negative), no | |
| | weeks, Suspected maternal | microarray | |
| | deletion; MR 5.652, 0.65 Mb | | |
| | deletion predicted | | |
| 8 | RI: Maternal age, testing at | CVS array, karyotype, and FISH | Maternal hypothyroid disorder |
| | 10 weeks, Suspected | (negative), no maternal testing | |
| | maternal deletion; MR and | | |
| | cfDNA size NA | | |
| 9 | RI: Maternal age, testing at | Postnatal array (negative), no | |
| | 15 weeks, Suspected | maternal testing | |
| | maternal deletion; MR | | |
| | 8.295, 2.35 Mb deletion | | |
| | predicted | | |
| 10 | RI: Maternal age, testing at | CVS array (negative), no | Atypical deletion predicted |
| | 11 weeks, Suspected | maternal testing | |
| | maternal deletion; MR | | |
| | 12.127, 0.65 Mb deletion | | |
| | predicted | | |
| 11 | RI: Ultrasound finding, | FISH/karyotype on amnio (both | 2VC, CPC, Club foot on ultrasound |
| | testing at 24 weeks, | negative), no microarray and no | |
| | Suspected maternal deletion; | maternal testing | |
| | MR and cfDNA size NA | | |
| 12 | RI: Ultrasound finding, | Karyotype on POC specimen | Ultrasound findings not specified; |
| | testing at 12 weeks, | after SAB, maternal balanced | Maternal phenotype reported with |
| | Suspected maternal deletion; | translocation confirmed | severe LD/DD, dysmorphic facies |
| | MR 13.38, 2.35 Mb deletion | (46, XX, t(16; 18)(q13; p11.2)mat), | reportedly consistent with 22g11.2DS |
| | | no microarray and no maternal | |
| | | microarray/FISH | |
| 13 | RI: Ultrasound finding, | FISH/karyotype on amnio (both | Bilateral CPCs |
| | testing at 22 weeks, | negative), no microarray and no | |
| | Suspected maternal deletion; | maternal testing | |
| | MR and cfDNA size NA | | |
| 14 | RI: Ultrasound finding, | Amnio karyotype (negative), no | CPCs |
| | testing at 18 weeks, | microarray and no maternal | |
| | Suspected maternal deletion; | testing | |
| | MR: 8.29, cfDNA size NA | | |
| 15 | RI: Ultrasound finding, | No fetal testing, Maternal FISH | IUGR; Atypical deletion predicted |
| | testing at 30 weeks, | only (negative), no microarray | |
| | Suspected maternal deletion; | | |
| | MR: 19.402, 0.65 Mb | | |
| | deletion | | |
| 16 | RI: None provided, testing at | No fetal testing, Maternal | Atypical deletion predicted |
| | 10 weeks, Suspected | karyotype only (negative), no | |
| | maternal deletion; MR: | microarray | |
| | 14.754, 0.65 Mb deletion | | |
| 17 | RI: No known high-risk | FISH/karyotype on amnio (both | Atypical deletion predicted |
| | indication, GA not provided, | negative), no microarray and no | |
| | suspected maternal deletion; | maternal testing | |
| | MR 15.49, 0.65 Mb deletion | | |

cfDNA = cell-free DNA,
RI = referral indication,
MR = mosaicism ratio,
Mb = megabase,
NA = not available,
TOF = tetralogy of fallot,
FISH = fluorescent in situ hybridization,
2VC = two vessel cord,
CPC = choroid plexus cyst(s),
LD/DD = learning difficulties/developmental delay,
22q11.2DS = 22q11.2 deletion syndrome,
IUGR = intrauterine growth restriction,
GA = gestational age

TABLE 3
PPV calculations

| | True | False | |
|---|---|---|---|
| | positive | Positive | PPV |
| Total cases with complete diagnostic | 176 | 1 | 99.4% |
| testing (n = 177) | | | (95% CI: |
| 75 suspected fetal cases with fetal | | | 96.4%-99.9%) |
| testing (74 TP, 1 FP) | | | |
| 97 suspected maternal cases with | | | |
| maternal testing (97 TP) | | | |
| 5 suspected maternal cases with only | | | |
| fetal testing (5 TP) | | | |
| Treating ‘incomplete’ as FP (n = 194) | 176 | 18 | 90.7% |
| | | | (95% CI: |
| | | | 85.5%-94.2%) |
| Cases with fetal testing + fetus suspected | 74 | 1 | 98.7% |
| (minus maternal suspected cases) (n = 75) | | | (95% CI: |
| | | | 91.8%-99.9%) |
| Also including cases without diagnostic | | | |
| testing (n = 307) as either: | | | |
| All FP | 176 | 131 | 57.3% |
| | | | (95% CI: |
| | | | 51.6%-62.9%) |
| All TP | 306 | 1 | 99.7% |
| | | | (95% CI: |
| | | | 97.9%->99.9%) |

PPV = positive predictive value,
FP = false positive,
TP = true positive

If the cases without diagnostic testing were factored into the PPV calculations, an upper and lower bound to PPV can be calculated as included in Table 3. If all cases without complete diagnostic testing were included and treated as true positives, the upper bound to the PPV would be 99.7%. If all cases without complete diagnostic testing were treated as false positives, the lower bound to the PPV would be 57.3%.

There was one false positive case in a patient screened at 12 weeks of gestation with the indication of advanced maternal age in a singleton pregnancy. The data suggested a fetal deletion, with an overall fetal fraction of 4.43%, an MR of 1.496, and an estimated deletion size of 2.75 Mb on cfDNA. FISH and microarray via amniocentesis were reportedly normal; no maternal testing was completed.

Table 4 summarizes the diagnostic testing available for this cohort. As seen in the supplement, the most common assay/specimen combinations were maternal blood microarray (n=58), amniotic fluid microarray (n=35), and postnatal/neonatal microarray (n=31). Of the cases with fetal/neonatal testing (n=118), 45.8% (n=54) deferred testing to the postnatal period or on a POC specimen following a loss.

TABLE 4
Details of diagnostic testing specimens and assays

| | |
|---|---|
| No diagnostic testing | 113 |
| Confirmation in fetus/baby (specimen and assay type unspecified) | 9 |
| CVS FISH | 4 |
| CVS karyotype | 2 |
| CVS microarray | 6 |
| Amniotic fluid FISH | 8 |
| Amniotic fluid karyotype | 13 |
| Amniotic fluid microarray | 35 |
| Amniotic fluid - assay type unspecified | 1 |
| POC FISH | 1 |
| POC karyotype | 2 |
| POC microarray | 3 |
| Postnatal (Neonatal) FISH | 12 |
| Postnatal (Neonatal) microarray | 31 |
| Postnatal (Neonatal) - assay type unspecified | 10 |
| Maternal FISH | 21 |
| Maternal karyotype | 6 |
| Maternal microarray | 58 |
| Maternal testing - assay type unspecified | 28 |

Note that most cases (n = 137) had only one diagnostic test on a single specimen type, however, there were 48 cases that involved testing on either multiple assays or specimens (for example, both maternal and fetal microarray or both FISH and microarray on an amniotic fluid specimen), so the counts in the table are not mutually exclusive.
CVS = chorionic villus sampling,
FISH = fluorescence in situ hybridization,
POC = products of conception

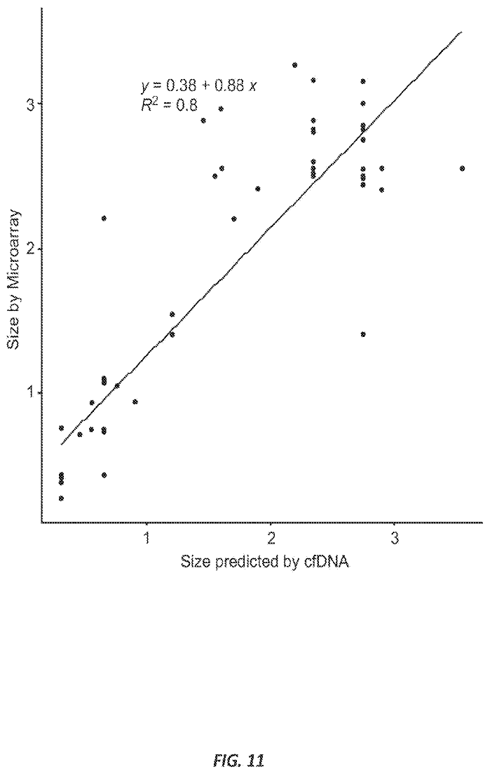

For the 89 cases with deletion sizes available from microarray confirmation, the median deletion size was 2.48 Mb (range 0.268 Mb-3.26 Mb). Predicted fetal confirmed deletions (n=37) had a median size of 2.55 Mb (range 0.93 Mb-3.26 Mb) and predicted maternal confirmed deletions (n=52) had a median size of 0.75 Mb (range 0.268 Mb-3.160 Mb). Similar to the predicted sizes by cfDNA, there is a significant difference (p=2.728e−06) in the size of deletions on array for those that were predicted to be maternal events versus fetal events from cfDNA. For cases with a predicted size available from cfDNA and confirmed size available from microarray, FIG. 11 shows the relationship between the estimated size and the actual size. Using linear regression, sizes were highly concordant between array and cfDNA with an r-square of 0.8.

When a deletion of fetal origin was suspected (n=130), complete fetal testing was available for 75 cases. Of those, 74 had a fetal deletion confirmed; the other was a false positive with no maternal testing. Four of the suspected fetal cases with diagnostic confirmation also had maternal results available, all of which were normal. One of these ‘fetal’ cases was noted to be paternally inherited based on a known deletion in the family.

When a maternal deletion was suspected (n=177), testing was available for 105 cases, however, complete maternal testing was available for 97 cases (an additional 8 had ‘incomplete’ maternal testing). Of those 97, a maternal deletion was confirmed in all cases. Of note, of the 97 cases with a confirmed maternal deletion, 35 had knowledge of the maternal deletion prior to testing, while 63.9% presumably did not based on the outcome collection notes. Of these 35 cases, 2 reported a maternal phenotype. The remaining 33 did not report a maternal phenotype to the laboratory, although this does not preclude the presence of findings that were simply not reported to the lab. The remaining 22 cases with a maternal phenotype reported were presumably not known to have a 22q11.2DS prior to testing.

Additionally, there were 43 suspected maternal cases that had fetal testing available (31/35 from the ‘testing in both’ group and 12/83 from the ‘fetal testing only’ group). Of those 43 cases, a fetal deletion was found in 58.1% cases (n=25, 5/12 from the ‘fetal testing only’ group and 20/31 from the ‘testing in both’ group), confirming fetal inheritance of the suspected maternal deletion. For the 113 cases without diagnostic testing, Table 5 summarizes the indications for testing and provides details about ultrasound findings, where available, as well as any notes about pregnancy outcome. Similar to the overall cohort, approximately half of cases without diagnostic testing had ultrasound findings noted.

TABLE 5
Clinical details of cases without diagnostic testing.

| | | Suspected | | | |
|---|---|---|---|---|---|
| | | origin on | | | |
| | | cfDNA | Ultrasound | | |
| Case | Screening | sequencing | findings | | |
| Identifier | indication | data | reported? | If yes, details: | Notes |
| 6 | Maternal age, | Fetal | Y | NOS | |
| | Ultrasound | | | | |
| | finding | | | | |
| 11 | Ultrasound | Fetal | Y | TOF, pyelectasis | Patient moved, no follow-up |
| | finding | | | | |
| 18 | Personal or | Fetal | N | | |
| | family history | | | | |
| 25 | Ultrasound | Fetal | Y | IUGR | |
| | finding | | | | |
| 33 | Abnormal serum | Maternal | Y | Truncus arteriosus and | |
| | biochemical | | | renal anomalies | |
| | screening, | | | | |
| | Ultrasound | | | | |
| | finding | | | | |
| 35 | Ultrasound | Fetal | Y | IUGR, cleft lip | |
| | finding | | | | |
| 37 | Maternal age | Maternal | N | | Declined testing |
| 38 | Ultrasound | Fetal | Y | NOS | LTFU |
| | finding | | | | |
| 40 | Ultrasound | Maternal | Y | Anhydramnios and renal | TOP, no POC testing |
| | finding | | | anomalies | |
| 41 | No known | Maternal | N | | LTFU |
| | high risk | | | | |
| 43 | Ultrasound | Fetal | Y | TOF | |
| | finding | | | | |
| 44 | Maternal age | Maternal | N | | |
| 47 | Maternal age, | Maternal | N | | |
| | Abnormal serum | | | | |
| | biochemical | | | | |
| | screening | | | | |
| 57 | Maternal age | Maternal | N | | |
| 58 | Maternal age | Maternal | N | | |
| 59 | Maternal age | Maternal | N | | TOP, no testing |
| 60 | Maternal age | Maternal | N | | |
| 62 | Ultrasound | Fetal | Y | Polyhydramnios | |
| | finding | | | | |
| 64 | Abnormal serum | Maternal | N | | |
| | biochemical | | | | |
| | screening | | | | |
| 68 | None provided | Maternal | N | | |
| 72 | Maternal age | Maternal | N | | |
| 75 | Maternal age | Maternal | N | | |
| 78 | Ultrasound | Fetal | Y | Polyhydramnios, cleft | |
| | finding | | | palate, VSD | |
| 79 | Ultrasound | Fetal | Y | Complex heart defect | |
| | finding | | | | |
| 82 | Maternal age | Fetal | N | | |
| 86 | Ultrasound | Maternal | Y | Heart defect | |
| | finding, | | | NOS | |
| | Maternal age | | | | |
| 91 | Abnormal serum | Maternal | Y | NOS | |
| | biochemical | | | | |
| | screening, | | | | |
| | Ultrasound | | | | |
| | finding | | | | |
| 93 | Maternal age | Maternal | N | | |
| 94 | Ultrasound | Maternal | Y | Multiple congenital | cfDNA also positive |
| | finding | | | anomalies | for trisomy 21 |
| 95 | Maternal age | Maternal | N | | |
| 97 | Ultrasound | Maternal | Y | NOS | |
| | finding | | | | |
| 98 | Ultrasound | Fetal | Y | NOS | |
| | finding | | | | |
| 105 | Ultrasound | Maternal | Y | IUGR | |
| | finding | | | | |
| 111 | Maternal age | Maternal | N | | |
| 112 | None provided | Maternal | N | | |
| 116 | Ultrasound | Fetal | Y | VSD | |
| | finding | | | | |
| 120 | Personal or | Maternal | N | | |
| | family history | | | | |
| 125 | Maternal age | Maternal | N | | |
| 127 | Ultrasound | Fetal | Y | Truncus arteriosus, VSD | |
| | finding | | | | |
| 128 | Ultrasound | Fetal | Y | Fetal cardiac anomalies | |
| | finding | | | NOS, pulmonary stenosis / | |
| | | | | VSD/ ? aortic stenosis | |
| 135 | Ultrasound | Fetal | Y | EIF and 2VC | |
| | finding | | | | |
| 137 | Maternal age | Fetal | N | | |
| 139 | Ultrasound | Fetal | Y | 2VC, left SVC, SGA | |
| | finding | | | | |
| 140 | Ultrasound | Fetal | Y | TOF | |
| | finding | | | | |
| 142 | Ultrasound | Fetal | Y | Heart defect NOS and | |
| | finding | | | polyhydramnios | |
| 144 | Ultrasound | Fetal | Y | Truncus arteriosus | |
| | finding | | | | |
| 145 | Ultrasound | Fetal | Y | Heart defect | |
| | finding | | | NOS | |
| 146 | Ultrasound | Fetal | Y | Heart defect | |
| | finding | | | NOS and renal anomalies | |
| 148 | Maternal age | Maternal | N | | Egg donor gestation so fetus |
| | | | | | not at risk |
| 151 | Ultrasound | Fetal | Y | Heart defect | |
| | finding | | | NOS, IUFD | |
| 152 | Maternal age | Maternal | N | | |
| 155 | Maternal age | Maternal | N | | |
| 158 | None provided | Fetal | N | | |
| 160 | Maternal age | Maternal | N | | |
| 164 | Ultrasound | Fetal | Y | TOF | |
| | finding | | | | |
| 167 | Maternal age | Maternal | N | | |
| 168 | Maternal age | Maternal | N | | |
| 170 | Ultrasound | Maternal | Y | CPC, EIF | |
| | finding | | | | |
| 172 | No known | Maternal | N | | |
| | high risk | | | | |
| 174 | Maternal age | Fetal | N | | |
| 177 | Maternal age | Maternal | N | | |
| 178 | Maternal age | Fetal | N | | |
| 179 | Ultrasound | Fetal | Y | Heart defect | |
| | finding | | | NOS | |
| 180 | Ultrasound | Fetal | Y | Heart defect | |
| | finding | | | NOS | |
| 181 | Maternal age | Fetal | N | | |
| 182 | Ultrasound | Fetal | Y | NOS | |
| | finding | | | | |
| 184 | No known | Fetal | Y | Right sided aortic arch | |
| | high risk | | | with vascular ring | |
| 187 | Ultrasound | Fetal | Y | VSD; small aortic valve | Negative maternal karyotype |
| | finding | | | and arch | |
| 188 | Ultrasound | Fetal | Y | TOF | |
| | finding | | | | |
| 190 | Maternal age | Maternal | N | | Prior child with DiGeorge |
| | | | | | (unclear whether diagnosis |
| | | | | | based on clinical features |
| | | | | | or confirmed with testing) |
| 191 | Ultrasound | Fetal | Y | TOF | LTFU |
| | finding | | | | |
| 194 | Maternal age | Maternal | N | | |
| 198 | Abnormal serum | Maternal | N | | Prior child with DiGeorge |
| | biochemical | | | | (unclear whether diagnosis |
| | screening, | | | | based on clinical features |
| | Personal or | | | | or confirmed with testing) |
| | family history | | | | |
| 199 | Ultrasound | Fetal | Y | Truncus arteriosus, | |
| | finding | | | IUGR | |
| 200 | Ultrasound | Fetal | Y | TOF | |
| | finding | | | | |
| 203 | Ultrasound | Fetal | Y | Heart defect | |
| | finding | | | NOS | |
| 204 | Ultrasound | Fetal | Y | Unilateral club foot; | |
| | finding | | | sandle-gap toe; | |
| | | | | polyhydramnios; | |
| | | | | didn't visualize stomach. | |
| 206 | Ultrasound | Fetal | Y | Complex cardiac defect | |
| | finding | | | | |
| 211 | Ultrasound | Maternal | Y | Heart defect | |
| | finding | | | NOS; echogenic lungs | |
| 212 | Ultrasound | Maternal | Y | EIF left ventricle | |
| | finding | | | | |
| 217 | None provided | Maternal | N | | |
| 219 | No known | Maternal | N | | |
| | high risk | | | | |
| 222 | No known | Maternal | N | | Patient tested with subsequent |
| | high risk | | | | pregnancy with similar findings |
| 223 | Personal or | Maternal | N | | |
| | family history | | | | |
| 229 | Ultrasound | Maternal | Y | Bilateral renal agenesis | |
| | finding | | | | |
| 231 | Ultrasound | Fetal | Y | TOF | |
| | finding | | | | |
| 233 | Maternal age | Maternal | N | | |
| 236 | Ultrasound | Maternal | Y | NOS | Patient had TOP; |
| | finding | | | | Severe diabetic |
| 239 | Ultrasound | Fetal | Y | TOF, absent pulmonary | |
| | finding, | | | valves | |
| | Abnormal serum | | | | |
| | biochemical | | | | |
| | screening | | | | |
| 240 | Ultrasound | Fetal | Y | EIF, absent nasal bone, | |
| | finding | | | aortic arch | |
| 243 | Ultrasound | Fetal | Y | TOF and major | |
| | finding | | | aortopulmonary | |
| | | | | collateral anastomoses | |
| 252 | Ultrasound | Fetal | Y | VSD and aortic stenosis | |
| | finding | | | | |
| 257 | Personal or | Maternal | N | | |
| | family history | | | | |
| 258 | Ultrasound | Fetal | Y | NOS | |
| | finding | | | | |
| 263 | Maternal age | Maternal | N | | |
| 265 | No known | Fetal | N | | |
| | high risk | | | | |
| 266 | Maternal age, | Fetal | Y | Heart defect | |
| | Ultrasound | | | NOS | |
| | finding | | | | |
| 270 | Ultrasound | Maternal | Y | Diaphragmatic hernia | |
| | finding | | | | |
| 274 | Maternal age | Fetal | N | | |
| 277 | No known | Maternal | N | | |
| | high risk | | | | |
| 282 | Ultrasound | Fetal | Y | Heart defect | |
| | finding | | | NOS | |
| 283 | None provided | Maternal | N | | Declined testing |
| 285 | No known | Maternal | N | | |
| | high risk | | | | |
| 286 | None provided | Maternal | N | | |
| 289 | Ultrasound | Maternal | Y | NOS | |
| | finding | | | | |
| 291 | Ultrasound | Fetal | Y | NOS | |
| | finding | | | | |
| 295 | No known | Maternal | N | | Normal ultrasound at 15 weeks; |
| | high risk | | | | patient declined testing |
| 297 | No known | Maternal | N | | |
| | high risk | | | | |
| 299 | None provided | Maternal | N | | |
| 301 | None provided | Maternal | N | | Patient declined testing, normal |
| | | | | | anatomy scan |
| 304 | No known | Maternal | N | | |
| | high risk | | | | |
| 305 | Ultrasound | Fetal | Y | NOS | |
| | finding | | | | |
| 307 | No known | Maternal | N | | |
| | high risk | | | | |

TOF = tetralogy of fallot,
VSD = ventricular septal defect,
NOS = Not otherwise specified,
IUGR = intrauterine growth restriction,
EIF = echogenic intracardiac focus,
NB = nasal bone,
CNS = central nervous system,
2VC = two vessel cord,
NT = nuchal translucency,
CPCs = choroid plexus cysts,
LFTU = lost to follow-up,
TOP = termination of pregnancy,
SGA = small for gestational age,
SVC = superior vena cava,
IUFD = intrauterine fetal demise

### (4) Ultrasound Findings

As noted above, 157 cases (51.14%) were noted to have ultrasound findings either at the time of testing or at some point during the pregnancy. Although the degree of detail regarding the specific ultrasound findings varied (for example, 19 cases had no further details about the ultrasound findings), most cases contained some additional information about the findings. Table 6 contains the summary details of the ultrasound findings across these 157 cases, as available. Table 7 lists the 75 suspected fetal cases with diagnostic testing (74 true positives and 1 false positive) with the ultrasound finding information amended.

TABLE 6
Summary of the available details for cases
with reported ultrasound findings

| | |
|---|---|
| Cardiac (Isolated) | 71 |
| TOF | 22 |
| VSD | 7 |
| Truncus arteriosus | 4 |
| Aortic valve stenosis | 1 |
| Complex or multiple cardiac anomaly | 15 |
| Unspecified cardiac anomaly | 20 |
| Cardiac + Renal | 6 |
| TOF + Pyelectasis | 1 |
| Truncus arteriosus + Renal Anomalies NOS | 1 |
| Right aortic arch with vascular ring + | 1 |
| Multicystic dysplastic kidney | |
| Cardiac anomaly NOS + Pyelectasis | 1 |
| Cardiac anomaly NOS + Renal anomalies NOS | 1 |
| Cardiac anomaly NOS + Cystic kidney + | 1 |
| Umbilical vein varix | |
| Cardiac + Additional findings (Non-Renal) | 18 |
| With facial cleft (Cardiac anomaly NOS ×1) | 1 |
| With fetal demise (Ductus arteriosus ×1, | 2 |
| Cardiac anomaly NOS ×1) | |
| With polyhydramnios (TOF ×2, Cardiac anomaly NOS ×2) | 4 |
| With IUGR (Truncus arteriosus ×1, Cardiac anomaly NOS ×1) | 2 |
| With echogenic lungs (Cardiac anomaly NOS ×1) | 1 |
| With soft markers (Coarctation of the aorta/EIF ×1, | 2 |
| Aortic arch anomaly/EIF/Absent NB ×1) | |
| Cardiac anomaly NOS + Shortened long bones + | 1 |
| CNS anomaly NOS + Absent NB | |
| TOF + Absent thymus + Low set ears | 1 |
| VSD + Polyhydramnios + Cleft palate | 1 |
| Complex cardiac anomaly + Suspected esophageal | 1 |
| atresia + Polyhydramnios | |
| Left-sided superior vena cava + 2VC + IUGR | 1 |
| Complex cardiac anomaly + 2VC + | 1 |
| Bilateral clubbed feet | |
| IUGR | 11 |
| Isolated IUGR | 7 |
| With cleft lip | 1 |
| With duodenal atresia | 1 |
| With stillbirth | 1 |
| With soft markers (EIF + 2VC) | 1 |
| Fluid | 4 |
| Polyhydramnios | 4 |
| Lymphatic | 3 |
| Increased NT | 2 |
| Cystic hygroma + hydrops | 1 |
| Nervous system | |
| Neural tube defect | |
| NOS | 19 |
| Other Structural Anomaly | 3 |
| Omphalocele | |
| Diaphragmatic hernia | |
| Renal | 7 |
| Pyelectasis | |
| Bilateral renal agenesis | |
| Anhydramnios + Renal anomalies NOS | |
| Oligohydramnios + Absent right kidney | |
| Unilateral renal agenesis + Unilateral club foot | |
| Bilateral hydronephrosis + Clubbed feet | |
| Multicystic kidney + Unilateral urinary tract | |
| dilatation + Bilateral CPCs | |
| Skeletal | 2 |
| Unilateral clubfoot | |
| Bilateral clubbed feet + Polyhydramnios | |
| Soft markers | 7 |
| CPCs | |
| Absent NB | |
| EIF + 2VC | |
| CPC + EIF | |
| EIF | |
| EIF + Echogenic bowel + Fetal demise | |
| Multisystem (non-cardiac) | 6 |
| 2VC + CPC + Clubfoot | |
| MCA NOS | 3 |
| Polyhydramnnios + Abnormal ear + Clinodactyly | |
| Polyhydramnios + Unilateral clubbed foot + | |
| Sandal-gap toe + Unable to see stomach | |

TOF = tetralogy of fallot,
VSD = ventricular septal defect,
NOS = Not otherwise specified,
IUGR = intrauterine growth restriction,
EIF = echogenic intracardiac focus,
NB = nasal bone,
CNS = central nervous system,
2VC = two vessel cord,
NT = nuchal translucency,
CPCs = choroid plexus cysts,
MCA = multiple congenital anomalies

TABLE 7
Ultrasound details for suspected fetal cases with diagnostic testing
(74 true positive fetal cases and 1 false positive case)

| | Ultrasound | | |
|---|---|---|---|
| Case | findings | | |
| Identifier | reported? | If yes, details: | Classification |
| 1 | Y | TOF | True Positive |
| 2 | Y | TOF | True Positive |
| 5 | Y | Heart defect (unspecified) | True Positive |
| 10 | Y | Heart defect (unspecified), | True Positive |
| | | shortened long bones, | |
| | | CNS abnormality (unspecified), | |
| | | absent nasal bone | |
| 13 | Y | TOF | True Positive |
| 21 | Y | Thick NT | True Positive |
| 22 | Y | Myelomeningocele | True Positive |
| 26 | Y | VSD, coarctation of the aorta | True Positive |
| 30 | Y | NOS | True Positive |
| 34 | Y | NOS | True Positive |
| 36 | Y | Heart defect (unspecified) | True Positive |
| 39 | Y | VSD | True Positive |
| 42 | Y | IUGR | True Positive |
| 49 | N | | True Positive |
| 51 | Y | Heart defect (unspecified) | True Positive |
| 61 | Y | Polyhydramnios, heart defect | True Positive |
| | | (unspecified) | |
| 65 | Y | VSD | True Positive |
| 66 | Y | Pyelectasis | True Positive |
| 67 | N | | True Positive |
| 70 | N | | True Positive |
| 74 | Y | IUGR, duodenal atresia | True Positive |
| 80 | Y | Heart defect (unspecified) | True Positive |
| 81 | Y | TOF | True Positive |
| 83 | Y | Heart defect (unspecified) | True Positive |
| 87 | Y | Truncus arteriosus | True Positive |
| 89 | Y | Polyhydramnios | True Positive |
| 90 | Y | TOF | True Positive |
| 100 | Y | Heart defect (unspecified) | True Positive |
| 101 | Y | Cystic hygroma, hydrops | True Positive |
| 102 | Y | Coarctation of aorta and EIF | True Positive |
| 104 | Y | Right aortic arch, vascular ring, | True Positive |
| | | multicystic dysplastic kidney | |
| 107 | Y | VSD | True Positive |
| 110 | N | | True Positive |
| 113 | Y | Unilateral renal agenesis, | True Positive |
| | | unilateral club foot | |
| 115 | Y | Echogenic bowel, EIF, IUFD | True Positive |
| 117 | Y | NOS | True Positive |
| 118 | Y | Ductus arteriosus; infant demise | True Positive |
| 126 | Y | Heart defect and pyelectasis | True Positive |
| 129 | Y | TOF | True Positive |
| 130 | Y | 2VC, complex heart defect | True Positive |
| | | (unspecified), | |
| | | polyhydramnios, esophageal | |
| | | atresia?, perinatal demise | |
| 131 | Y | Heart defect (unspecified) | True Positive |
| 132 | N | | True Positive |
| 133 | N | | True Positive |
| 136 | Y | Heart defect (unspecified); IUGR | True Positive |
| 138 | N | | True Positive |
| 141 | Y | Unilateral clubfoot | True Positive |
| 147 | N | | True Positive |
| 154 | Y | Omphalocele | True Positive |
| 156 | Y | Polyhydramnios, abnormal ear, | True Positive |
| | | clinodactyly | |
| 161 | Y | TOF, polyhydramnios | True Positive |
| 165 | Y | Bilateral clubbed feet, 2VC, | True Positive |
| | | VSD, possible transposition | |
| | | of great arteries | |
| 169 | Y | Heart defect (unspecified) | True Positive |
| 171 | N | | False Positive |
| 173 | N | | True Positive |
| 175 | N | | True Positive |
| 176 | Y | TOF | True Positive |
| 186 | N | | True Positive |
| 202 | Y | Cystic kidney; umbilical vein varix; | True Positive |
| | | possible heart defect (unspecified) | |
| 208 | Y | Complex heart defect (unspecified) | True Positive |
| 218 | Y | Complex heart defect (unspecified) | True Positive |
| 220 | Y | TOF | True Positive |
| 224 | Y | TOF | True Positive |
| 232 | Y | TOF | True Positive |
| 237 | Y | TOF with right aortic arch | True Positive |
| 238 | Y | Heart defect (unspecified) | True Positive |
| 247 | Y | TOF | True Positive |
| 249 | Y | Heart defect (unspecified), cleft lip | True Positive |
| 254 | Y | IUGR, 2VC, EIF | True Positive |
| 261 | N | | True Positive |
| 262 | Y | Severe polyhydramnios | True Positive |
| 267 | Y | Bilateral clubbed feet and | True Positive |
| | | polyhydramnios | |
| 269 | Y | Heart defect (unspecified) | True Positive |
| 276 | Y | VSD, vascular ring and aberrant | True Positive |
| | | right subclavian artery | |
| 278 | Y | TOF | True Positive |
| 292 | Y | VSD | True Positive |

TOF = tetralogy of fallot,
VSD = ventricular septal defect,
NOS = Not otherwise specified,
IUGR = intrauterine growth restriction,
EIF = echogenic intracardiac focus,
NB = nasal bone,
CNS = central nervous system,
2VC = two vessel cord,
NT = nuchal translucency

Cardiac findings were by far the most frequent anomaly reported on ultrasound (95/157, 60.5%), with 71 isolated cardiac anomalies, and 24 cases of cardiac anomalies accompanied by extracardiac anomalies. The most commonly reported cardiac anomalies were Tetralogy of Fallot (TOF), ventricular septal defect (VSD), and truncus arteriosus. Complex cardiac anomalies were reported in isolation in 15 cases and with other extracardiac anomalies in 2 cases. Unspecified cardiac anomalies were reported in isolation in 20 cases and with other extracardiac anomalies in 10 cases. A handful of other specific cardiac anomalies were reported, as detailed in Table 8. There were 13 cases of reported renal findings on ultrasound; six of those were seen in conjunction with cardiac anomalies. Abnormalities of amniotic fluid were reported in a group of cases; polyhydramnios was seen as the only finding in 4 cases and in conjunction with other findings in 8 cases; oligo/anhydramnios was reported in 2 cases. Fetal growth concerns, either growth restriction (n=13) or shortened long bones (n=1), were reported frequently, as well. Oral clefts were only seen in two cases.

TABLE 8
Details of the cases with additional findings from microarray

| Case | Assay details | Additional array finding(s) |
|---|---|---|
| Case S1 | Maternal blood array | 1.61 Mb interstitial duplication of 9q33.1; |
| | | 570 kb interstitial duplication of 2p16.3 |
| Case S2 | POC + cord blood array | 1.33 Mb interstitial duplication of 17p12 |
| Case S3 | Maternal blood array | 853 kb interstitial duplication of Xp22.2 |
| Case S4 | Maternal blood array | 700 kb gain of 4p16.1 |
| Case S5 | Maternal blood array | 853 kb interstitial duplication of Xp22.2 |
| Case S6 | Amniotic fluid array | 546 kb interstitial duplication of 15q26.3 |
| Case S7 | Maternal blood array | 855 kb interstitial deletion of 15q11.2 |
| Case S8 | Maternal blood array | 472 kb interstitial duplication of 12q23.2 |
| Case S9 | Neonatal array | 255 kb interstitial deletion of 20p12.1 |
| Case S10 | Maternal blood array | 667 kb interstitial duplication of 6q27 |
| Case S11 | Neonatal array | 255 kb interstitial deletion of 6p25.1 |
| Case S12 | Maternal blood array | 94 kb interstitial deletion of 9p24.1 |
| Case S13 | Maternal blood array | “Small duplication on 15q” - verbal report from provider |
| Case S14 | Maternal blood array | 2.09 Mb interstitial duplication of 20q13.33 |
| Case S15 | Maternal blood array | 667 kb interstitial duplication of 6q27 |
| Case S16 | Maternal blood array | 22 kb interstitial deletion of 14q31.3 |

POC = products of conception,
Mb = Megabases,
kb = kilobases

### d) Discussion

The current study reflects on a number of considerations for prenatal screening for 22q11.2DS by cfDNA via massively parallel sequencing, as observed through the lens of the clinical experience of a single laboratory. These considerations are important for patient counseling prior to 22q11.2DS screening, but are especially critical for patients who receive a screen positive result.

In this cohort, the PPVs of approximately 91-99% were consistent with those reported for the MPS methodology and higher than those reported by SNP-based cfDNA methodologies of approximately 16% under the original protocol to 53% in a more recent study. This study includes a larger number of screen positive cases with diagnostic testing comparing with some prior or existing experiments. Another study using a targeted methodology reported no false positive cases for 22q11.2DS. The higher PPVs seen in the current study may, in part, be influenced by the presence of ultrasound findings in roughly half of all screen positive cases, and the clear association between the presence of ultrasound findings and the likelihood of fetal diagnostic testing demonstrated in this cohort. Patients with ultrasound findings at any point in the pregnancy were significantly more likely to have fetal diagnostic testing results available. For patients with structural anomalies on ultrasound, professional societies recommend diagnostic testing with microarray, but some patients may be reticent and opt to pursue screening. Screening is not a substitute for diagnostic testing. Nearly half of patients in this cohort who pursued fetal/neonatal diagnostic testing deferred this testing until after the baby was delivered or until after a pregnancy loss.

While PPV is one important metric, there are a number of other important factors that should be considered after a screen positive result. Laboratory algorithms, such as MR, may be useful in predicting whether the deletion observed in the cfDNA sequencing data is more likely to be of maternal or fetal origin. Indeed, in this study, the proprietary algorithm (informed by the strength of the sequencing signal of the event as well as other metrics such as MR, Z-score, and estimated fetal fraction) was helpful in predicting whether the event was maternal versus fetal in origin. Assignment of maternal versus fetal origin on cfDNA was accurate in all true positive cases. Using MR alone with a cutoff of 3 would correctly classify the majority of cases as maternal or fetal, although one confirmed maternal case had an MR of 2.969. This pregnancy was tested at 34 weeks gestation and had a fetal fraction of 27.83%, which could have influenced the ‘affected’ fraction and diminished the effect of the maternal contribution. Prediction of a maternal event could help guide testing for the pregnant patient, if desired, and if confirmed, would convey a 50% risk for fetal inheritance. Of the maternal deletions with fetal testing, approximately 50% of cases theoretically should confirm a fetal deletion; in this study, we observed that ~58% of fetuses inherited the maternal deletion. This is likely enriched by cases with ultrasound findings, which were more likely to pursue fetal testing.

For some patients, incidental discovery of a previously unknown 22q11.2 deletion may be helpful in explaining symptoms or a previously unrecognized phenotype, while for others, this information may be unwelcome or anxiety producing. As seen in this cohort, of the 24 cases with a reported maternal phenotype, 22 were not known/reported to the lab to have a known 22q11.2 deletion. Conversely, for the 35 confirmed maternal cases known prior to cfDNA screening, a phenotype was only reported to the lab for two cases. Given the retrospective nature of this study, we cannot assume an absence of maternal phenotype in cases where one was not reported. There were only 12 suspected maternal cases that were lack of a maternal phenotype was confirmed by the provider. Pretest counseling for cfDNA, especially when 22q11.2DS is included, should include a discussion of the risks, benefits and limitations of testing, including the risk for false positives and false negatives, and the possibility of uncovering unexpected maternal findings. In this study, limited information about maternal phenotype was available; results around maternal phenotype in this study should be interpreted with caution and additional information about maternal phenotypes would be useful in further exploring the impact of these findings. This affords an area for future study. It should be noted that approximately one third of confirmed maternal cases in the current study were already known to the patient prior to undergoing cfDNA screening. Though the maternal 22q11.2 event precluded assessment of the fetus for that specific region, these patients were still able to receive unhindered fetal screening for the remainder of the conditions assessed by the assay.

CfDNA screening using MPS can identify a range of deletion sizes for 22q11.2DS and is able to estimate the size and predicted breakpoints of the deletion, as demonstrated in this study. In fact, the data from the cohort with cfDNA-predicted deletion size and microarray-confirmed deletion size showed that cfDNA estimates were highly concordant with the diagnostic confirmation. Though the size and breakpoints of 22q11.2 deletions predicted by MPS-based cfDNA closely align with diagnostic results, these data points should be considered estimates, as MPS groups cfDNA into 50 kb “bins”; this grouping of cfDNA by genomic regions means that a margin of error will exist for all size and breakpoint predictions. Diagnostic testing with microarray analysis is essential for confirmation of deletion size and for determining the gene content in the deleted region. In general, maternal deletions were significantly smaller than fetal deletions, both in predicted and actual size. However, for both maternal and fetal events, the smallest confirmed deletion was less than 1 Mb. Given the genotype-phenotype associations, this could be relevant for patient counseling.

CfDNA size/breakpoint estimation also has practical implications for choosing an appropriate follow-up test; atypical or nested deletions may not be confirmed by FISH testing and 22q11.2DS deletions are typically below the resolution of a karyotype. In this cohort, there were at least 5 cases in which FISH testing was reportedly negative but a deletion was found on microarray. Most commercial FISH probes (N25, TUPLE1/HIRA, and TBX1) hybridize between low copy number repeat sequences A and B, so deletions that do not span this region will not be detected using these typical FISH probes. As seen in FIG. 8, while most cases were predicted to span the region that would be captured by these FISH probes, many of the smaller nested/atypical deletions were not predicted to overlap the hybridization region for these probes. Use of microarray for diagnostic confirmation of cfDNA results is recommended to avoid false negative results that may occur from FISH testing.

Additionally, microarray may also uncover findings beyond the 22q11.2 deletion suggested by cfDNA. It is worth noting that 16 of the cases with microarray in this cohort reported an additional finding beyond the suspected 22q11.2 deletions. Most of these findings were on maternal arrays; details of the additional array findings are provided in Table 8. These additional findings, which would have been missed by FISH testing alone, may have clinical implications for the patient and/or fetus. There were also five cases with a negative FISH but a positive array, including one case with a deletion found on maternal array for which postnatal FISH was ‘incorrectly’ ordered for the baby (the maternal deletion would not be detected by the FISH probes utilized). To the provider's knowledge, this patient did not return for follow-up postnatal microarray for the child.

Lastly, cardiac anomalies were especially prevalent in this cohort, as expected. Oral clefting was only seen in two cases, but may be limited by the ability of ultrasound to detect palatal anomalies especially those isolated to the soft palate. One study estimates the sensitivity for prenatal diagnosis of cleft lip/palate to be 88% via evaluation of the upper lip and notes that the overall sensitivity of ultrasound to diagnosis oral clefting is depressed by lower detection rates of isolated cleft palate.

### e) Conclusion

When cfDNA screening is positive for 22q11.2 deletion syndrome, clinicians and patients are faced with several decisions about follow-up testing. Should testing involve just the fetus, or should the mother be evaluated, as well? What type of analysis is most appropriate on the diagnostic specimen? Will FISH be able to detect the predicted abnormality, or is microarray necessary? Data from massively parallel sequencing may help to answer these questions and guide appropriate follow up testing for screen positive patients.

Ultimately, the experiments show that MPS-based cfDNA screening for 22q11.2DS can be an effective screening tool, may distinguish maternal from fetal events, and can be used to estimate the size and predicted location of the deletion. Positive screening results should be confirmed by diagnostic testing with microarray analysis, and no irreversible management decisions should be made on the basis of screening results alone, consistent with professional society recommendations. Furthermore, counseling following a screen-positive result should include a discussion of the variability in clinical features, although there is some genotype-phenotype correlation noted. Indeed, even within this cohort, affected pregnant patients were discovered following prenatal screening who had not previously been identified. Collaboration and communication between the cfDNA screening laboratory and the healthcare provider, especially in the event of a screen positive result, can optimize follow-up care. Genetic counselors working within the laboratory can review the metrics reviewed in this study, such as predicted size and breakpoints, whether the sequencing data suggests a maternal or fetal event, and retrospective PPVs based on the laboratory's experience and considering the patient's phenotype or the presence of ultrasound findings. Given that 22q11.2DS is a relatively common chromosome condition and may have implications for the pregnant person and the fetus, the potential benefits to screening may outweigh the challenges in the setting of education and informed consent.

### G. ADDITIONAL CONSIDERATIONS

Although specific examples have been described, various modifications, alterations, alternative constructions, and equivalents are possible. Examples are not restricted to operation within certain specific data processing environments but are free to operate within a plurality of data processing environments. Additionally, although certain examples have been described using a particular series of transactions and steps, it should be apparent to those skilled in the art that this is not intended to be limiting. Although some flowcharts describe operations as a sequential process, many of the operations may be performed in parallel or concurrently. In addition, the order of the operations may be rearranged. A process may have additional steps not included in the figure. Various features and aspects of the above-described examples may be used individually or jointly.

Further, while certain examples have been described using a particular combination of hardware and software, it should be recognized that other combinations of hardware and software are also possible. Certain examples may be implemented only in hardware, or only in software, or using combinations thereof. The various processes described herein may be implemented on the same processor or different processors in any combination.

Where devices, systems, components or modules are described as being configured to perform certain operations or functions, such configuration may be accomplished, for example, by designing electronic circuits to perform the operation, by programming programmable electronic circuits (such as microprocessors) to perform the operation such as by executing computer instructions or code, or processors or cores programmed to execute code or instructions stored on a non-transitory memory medium, or any combination thereof. Processes may communicate using a variety of techniques including but not limited to conventional techniques for inter-process communications, and different pairs of processes may use different techniques, or the same pair of processes may use different techniques at different times.

Specific details are given in this disclosure to provide a thorough understanding of the examples. However, examples may be practiced without these specific details. For example, well-known circuits, processes, algorithms, structures, and techniques have been shown without unnecessary detail in order to avoid obscuring the examples. This description provides example examples only, and is not intended to limit the scope, applicability, or configuration of other examples. Rather, the preceding description of the examples will provide those skilled in the art with an enabling description for implementing various examples. Various changes may be made in the function and arrangement of elements.

The specification and drawings are, accordingly, to be regarded in an illustrative rather than a restrictive sense. It will, however, be evident that additions, subtractions, deletions, and other modifications and changes may be made thereunto without departing from the broader spirit and scope as set forth in the claims. Thus, although specific examples have been described, these are not intended to be limiting. Various modifications and equivalents are within the scope of the following claims.

In the foregoing specification, aspects of the disclosure are described with reference to specific examples thereof, but those skilled in the art will recognize that the disclosure is not limited thereto. Various features and aspects of the above-described disclosure may be used individually or jointly. Further, examples may be utilized in any number of environments and applications beyond those described herein without departing from the broader spirit and scope of the specification. The specification and drawings are, accordingly, to be regarded as illustrative rather than restrictive.

In the foregoing description, for the purposes of illustration, methods were described in a particular order. It should be appreciated that in alternate examples, the methods may be performed in a different order than that described. It should also be appreciated that the methods described above may be performed by hardware components or may be embodied in sequences of machine-executable instructions, which may be used to cause a machine, such as a general-purpose or special-purpose processor or logic circuits programmed with the instructions to perform the methods. These machine-executable instructions may be stored on one or more machine readable mediums, such as CD-ROMs or other type of optical disks, floppy diskettes, ROMs, RAMs, EPROMs, EEPROMs, magnetic or optical cards, flash memory, or other types of machine-readable mediums suitable for storing electronic instructions. Alternatively, the methods may be performed by a combination of hardware and software.

Where components are described as being configured to perform certain operations, such configuration may be accomplished, for example, by designing electronic circuits or other hardware to perform the operation, by programming programmable electronic circuits (e.g., microprocessors, or other suitable electronic circuits) to perform the operation, or any combination thereof.

While illustrative examples of the application have been described in detail herein, it is to be understood that the inventive concepts may be otherwise variously embodied and employed, and that the appended claims are intended to be construed to include such variations, except as limited by the prior art.

1.-45. (canceled)

46. A method for providing an origin of a genetic variation in a biological sample from a subject, comprising:
obtaining circulating cell free nucleic acid in the biological sample from the subject, wherein the biological sample has been identified as having the genetic variation, and wherein the circulating cell free nucleic acid comprises major nucleic acid and minor nucleic acid;
sequencing, using a massively parallel sequencing (MPS) platform, the circulating cell free nucleic acid to obtain sequencing data;
identifying, by a computing system, one or more metrics for the genetic variation based on the sequencing data, wherein the one or more metrics comprise a size of the genetic variation and/or a breakpoint of the genetic variation;
determining, by the computing system, a mosaicism ratio for the genetic variation in the biological sample, wherein the determining the mosaicism ratio comprises:
determining, by the computing system, a fraction of nucleic acid having the genetic variation in the circulating cell free nucleic acid based on the sequencing data;
determining, by the computing system, a fraction of the minor nucleic acid in the circulating cell free nucleic acid based on the sequencing data; and
generating, by the computing system, the mosaicism ratio using the fraction of nucleic acid having the genetic variation in the circulating cell free nucleic acid to the fraction of the minor nucleic acid in the circulating cell free nucleic acid;
classifying, by the computing system, the origin of the genetic variation based on the mosaicism ratio, wherein the origin of the genetic variation is major when the mosaicism ratio is greater than or equal to a first threshold, and the origin of the genetic variation is minor when the mosaicism ratio is less than a second threshold; and
generating, by the computing system, a report concerning a recommendation of a follow-up screening, wherein the recommendation is based on the one or more metrics and the origin of the genetic variation.

47. The method of claim 46, wherein the genetic variation is an aneuploidy, a genome-wide copy number variation, a single gene disorder, or a microdeletion.

48. The method of claim 46, wherein:
(i) the subject is a pregnant subject, the major nucleic acid is maternal nucleic acid, and the minor nucleic acid is fetal nucleic acid;
(ii) the subject is a transplant patient, the major nucleic acid is recipient nucleic acid, and the minor nucleic acid is donor nucleic acid; or
(iii) the subject is a tumor patient, the major nucleic acid is normal nucleic acid, and the minor nucleic acid is tumor nucleic acid.

49. The method of claim 46, wherein the first threshold is 3.

50. The method of claim 49, wherein when the origin of the genetic variation is classified to be maternal when the mosaicism ratio is greater than or equal to 3, and when the genetic variation is an atypical or nested deletion, the recommendation is (i) not to perform a fluorescent in situ hybridization (FISH) test, or (ii) to perform a microarray analysis test.

51. The method of claim 46, further comprising determining, by the computing system, a presence of the genetic variation by a particular screening method or based on the sequencing data.

52. The method of claim 46, further comprising administering a particular screening test to the subject based on the report.

53. The method of claim 46, further comprising enriching the minor nucleic acid in the biological sample before sequencing.

54. A system comprising:
one or more data processors; and
a non-transitory computer readable storage medium containing instructions which, when executed on the one or more data processors, cause the one or more data processors to perform actions including:
sequencing circulating cell free nucleic acid in a biological sample from a subject to obtain sequencing data, wherein the biological sample has been identified as having a genetic variation, and wherein the circulating cell free nucleic acid comprises major nucleic acid and minor nucleic acid;
identifying one or more metrics for the genetic variation based on the sequencing data, wherein the one or more metrics comprise a size of the genetic variation and/or a breakpoint of the genetic variation;
determining a mosaicism ratio for the genetic variation in the biological sample, wherein the determining the mosaicism ratio comprises:
determining a fraction of nucleic acid having the genetic variation in the circulating cell free nucleic acid based on the sequencing data;
determining a fraction of the minor nucleic acid in the circulating cell free nucleic acid based on the sequencing data; and
generating the mosaicism ratio using the fraction of nucleic acid having the genetic variation in the circulating cell free nucleic acid to the fraction of the minor nucleic acid in the circulating cell free nucleic acid;
classifying an origin of the genetic variation based on the mosaicism ratio, wherein the origin of the genetic variation is major when the mosaicism ratio is greater than or equal to a first threshold, and the origin of the genetic variation is minor when the mosaicism ratio is less than a second threshold; and
generating a report concerning a recommendation of a follow-up screening, wherein the recommendation is based on the one or more metrics and the origin of the genetic variation.

55. The system of claim 54, wherein the genetic variation is an aneuploidy, a genome-wide copy number variation, a single gene disorder, or a microdeletion.

56. The system of claim 54, wherein:
(i) the subject is a pregnant subject, the major nucleic acid is maternal nucleic acid, and the minor nucleic acid is fetal nucleic acid;
(ii) the subject is a transplant patient, the major nucleic acid is recipient nucleic acid, and the minor nucleic acid is donor nucleic acid; or
(iii) the subject is a tumor patient, the major nucleic acid is normal nucleic acid, and the minor nucleic acid is tumor nucleic acid.

57. The system of claim 54, wherein the first threshold is 3.

58. The system of claim 57, wherein when the origin of the genetic variation is classified to be maternal when the mosaicism ratio is greater than or equal to 3, and when the genetic variation is an atypical or nested deletion, the recommendation is (i) not to perform a fluorescent in situ hybridization (FISH) test, or (ii) to perform a microarray analysis test.

59. The system of claim 54, wherein the actions further include determining a presence of the genetic variation by a particular screening method or based on the sequencing data.

60. A computer-program product tangibly embodied in a non-transitory machine-readable storage medium, including instructions configured to cause one or more data processors to perform actions including:
sequencing circulating cell free nucleic acid in a biological sample from a subject to obtain sequencing data, wherein the biological sample has been identified as having a genetic variation, and wherein the circulating cell free nucleic acid comprises major nucleic acid and minor nucleic acid;
identifying one or more metrics for the genetic variation based on the sequencing data, wherein the one or more metrics comprise a size of the genetic variation and/or a breakpoint of the genetic variation;
determining a mosaicism ratio for the genetic variation in the biological sample, wherein the determining the mosaicism ratio comprises:
determining a fraction of nucleic acid having the genetic variation in the circulating cell free nucleic acid based on the sequencing data;
determining a fraction of the minor nucleic acid in the circulating cell free nucleic acid based on the sequencing data; and
generating the mosaicism ratio using the fraction of nucleic acid having the genetic variation in the circulating cell free nucleic acid to the fraction of the minor nucleic acid in the circulating cell free nucleic acid;
classifying an origin of the genetic variation based on the mosaicism ratio, wherein the origin of the genetic variation is major when the mosaicism ratio is greater than or equal to a first threshold, and the origin of the genetic variation is minor when the mosaicism ratio is less than a second threshold; and
generating a report concerning a recommendation of a follow-up screening, wherein the recommendation is based on the one or more metrics and the origin of the genetic variation.

61. The computer-program product of claim 60, wherein the genetic variation is an aneuploidy, a genome-wide copy number variation, a single gene disorder, or a microdeletion.

62. The computer-program product of claim 60, wherein:
(i) the subject is a pregnant subject, the major nucleic acid is maternal nucleic acid, and the minor nucleic acid is fetal nucleic acid;
(ii) the subject is a transplant patient, the major nucleic acid is recipient nucleic acid, and the minor nucleic acid is donor nucleic acid; or
(iii) the subject is a tumor patient, the major nucleic acid is normal nucleic acid, and the minor nucleic acid is tumor nucleic acid.

63. The computer-program product of claim 60, wherein the first threshold is 3.

64. The computer-program product of claim 63, wherein when the origin of the genetic variation is classified to be maternal when the mosaicism ratio is greater than or equal to 3, and when the genetic variation is an atypical or nested deletion, the recommendation is (i) not to perform a fluorescent in situ hybridization (FISH) test or (ii) to perform a microarray analysis test.

65. The computer-program product of claim 60, wherein the actions further include determining a presence of the genetic variation by a particular screening method or based on the sequencing data.
